(19) United States
(12) Patent Application Publication
(10) Pub. No.: US 2024/0217960 A1
(43) Pub. Date: Jul. 4, 2024

(54) TRIAZINE DERIVATIVE AS COVALENT INHIBITORS OF PI3K

(71) Applicant: UNIVERSITÄT BASEL, Basel (CH)

(72) Inventors: Matthias WYMANN, Bern (CH); Chiara BORSARI, Basel (CH)

(73) Assignee: UNIVERSITÄT BASEL, Basel (CH)

(21) Appl. No.: 18/554,473

(22) PCT Filed: Apr. 9, 2022
(86) PCT No.: PCT/EP2022/059548

§ 371 Date: Oct. 8, 2023

(30) Foreign Application Priority Data
Apr. 9, 2021 (EP) 21167749.7

(51) Int. Cl. C07D 413/14; A61K 31/5377

(52) U.S. Cl. CPC C07D 413/14; A61K 31/5377

(57) ABSTRACT

Kinase-targeted covalent inhibitors are usually irreversibly targeting noncatalytic cysteines in the ATP-binding site. These compounds are designed by directly introducing an electrophile on a reversible-inhibitor scaffold. Our invention relates to novel triazine compounds, containing chemical reactive groups (warheads), targeting a solvent exposed cysteine at >10 Å from the core reversible inhibitor. A variety of novel linkers have been designed and used to link the warhead with the reversible scaffold. We disclose a novel chemical space for drug-like covalent modifiers of phosphoinositide 3-kinase alpha (PI3Kα), an enzyme frequently altered in human malignancies. The invention relates to novel covalent inhibitors showing higher in vitro affinity, cellular potency and improved metabolic stability (rat liver microsomes). The compounds of the invention could be exploited as therapeutic agents and chemical probes useful for modulating cellular activities such as signal transduction, proliferation, differentiation and cell death.

### FIELD OF THE INVENTION

The invention relates to novel triazine compounds, containing chemical reactive groups (warheads), as therapeutic agents and chemical probes useful for modulating cellular activities such as signal transduction, proliferation, differentiation and cell death. The compounds of the invention modulate kinase activity, in particular that of phosphoinositide 3-kinase (PI3K).

### BACKGROUND OF THE INVENTION

Protein kinases participate in the signaling events and control cellular activation, growth, differentiation, survival and migration in response to extracellular mediators or stimuli including growth factors, cytokines or chemokines.

Increased protein kinase activities are involved in many diseases including cancer, inflammatory disorders, metabolic and immunological diseases. These can be caused either directly or indirectly by the failure of control mechanisms due to mutation(s), overexpression or inappropriate control of enzyme activity.

The phosphoinositide 3-kinase (PI3K) signaling pathway plays a key role in many cellular processes, including cell growth, proliferation and survival. The PI3K family is divided into three classes according to their amino acid sequences, homology and substrate specificity. Class I PI3Ks are activated downstream of cell surface receptors, including receptor protein tyrosine kinases (RTKs), G-protein-coupled receptors (GPCRs) and immunoglobulin receptors. Class IA PI3Ks are obligate heterodimers composed of a catalytic subunit (p110α, p110β, or p110δ) and an associated regulatory subunit (p85α, p85β, p50α, p55α, or p55γ). The class IB PI3Kγ operates downstream of GPCRs and consists of a catalytic subunit (p110γ) and an adapter subunit (p84 or p101). Cell surface receptors activate PI3K to produce PtdIns(3,4,5)P₃, which serves as a docking site for protein kinase B (PKB/Akt) and 3-phosphoinositide-dependent protein kinase 1 (PDK1). This results in phosphorylation of the kinase domain at two regulatory sites, Thr308 by PDK1 and Ser473 by mTOR complex 2 (mTORC2). Hyperactivation of the PI3K/mTOR pathway can occur at multiple levels of this signaling cascade, finally promoting cancer growth and progression. Loss or inactivation of the tumor suppressor Phosphatase and Tensin homolog (PTEN), mutation or amplification of cell surface receptors, as well as the presence of activating hotspot mutations in PIK3CA, play a key role in human carcinogenesis. In addition, overactivation of the PI3K/mTOR axis has been associated with resistance to multiple cancer treatments. Therefore, PI3K inhibitors are considered as valuable asset in cancer therapy.

A considerable effort has been dedicated to the development of drugs targeting PI3K signaling, and many of them are currently being evaluated in clinical trials. Selective PI3Kα inhibitors could be beneficial for PIK3CA-mutant tumors and PIK3CA-related overgrowth syndromes (PROS), minimizing on-target metabolic side effects of pan-PI3K inhibitors. BYL719/Alpelisib/PIKRAY from Novartis and GDC-0032/Taselisib from Genentech act as a reversible modifier and are claimed as PI3Kα selective inhibitors. However, at the concentration needed for in vivo experiments they are not able to discriminate between PI3K isoforms. Only one PI3Kα covalent inhibitor is currently available, dubbed CNX-1351, but it shows a limited in vitro and cellular potency, low aqueous solubility and metabolic instability.

The patent application describes certain triazine derivatives having PI3K inhibitory activity and acting as irreversible modifiers, and their use as pharmaceuticals. The compounds herein covered have significant advantages in terms of potency, metabolic stability, and drug-likeness properties as compared to CNX-1351. In addition, the patent application describes linkers for the development of covalent kinase inhibitors.

### SUMMARY OF THE INVENTION

A first aspect of the invention relates to a compound of formula (IV) or a prodrug, metabolite, tautomer, solvate or pharmaceutically acceptable salt thereof,

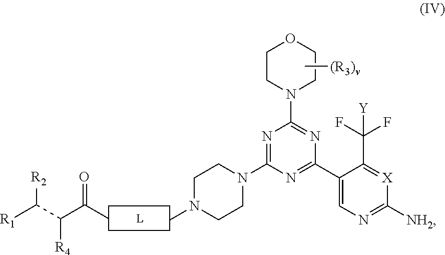

wherein
X is CH or N,
Y is H or F,
R₁ and R₂ are independently of each other selected from H, CH₃, cyclopropyl, —F, —CH₂—F, —CH₂—CH₂—F, —CN,

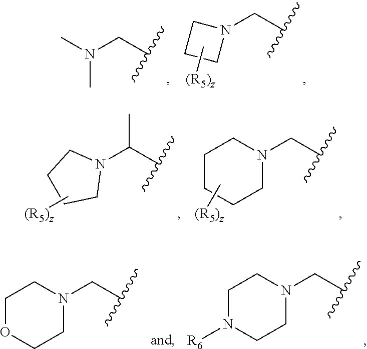

with R₅ being F or CH₃, R₆ being C₁₋₆-alkyl and z being 0, 1 or 2,
R₃ is C₁₋₃-alkyl or two residues R₃ form a bridge —(CH₂)ᵣ — with r being 1, 2 or 3,
v is 0, 1, 2, 3 or 4,
R₄ is H, F or —CN,
L is a linker moiety composed of C, N, O and/or H atoms, wherein the linker moiety L has a length between XX Å and XX Å,
the dashed line represents a carbon-carbon single bond or carbon-carbon double bond.

A second aspect of the invention relates to the compound according to the first aspect of the invention for use in the treatment of a disease.

A third aspect of the invention relates to the compound according to the first aspect of the invention for use in the treatment of tumor disease, overgrowth syndrome, neurological disease disorder, immunological disease disorder.

A fourth aspect of the invention relates to an intermediate of formula V,

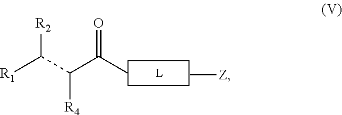

wherein
R₁, R₂, R₄ and L are defined as described above,
Z is —OH, Br, COOH or NH₂.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials herein described.

### Definitions

The term “alkyl” as used herein refers to a saturated linear-chain monovalent hydrocarbon group of one to five carbon atoms (C₁-C₅). Examples of alkyl groups include, but are not limited to, methyl, ethyl, 1-propyl (n-propyl), 1-butyl (n-butyl).

The terms “heterocycle”, “heterocyclyl” and “heterocyclic ring” are used interchangeably herein and refer to a saturated or unsaturated carbocyclic radical of 4 to 6 ring atoms in which at least one ring atom is a heteroatom, particularly nitrogen, and the remaining ring atoms being carbon atoms, wherein one or more ring atoms are optionally substituted independently with one or more substituents, particularly selected from —CH₃ and —F.

The term “chiral” refers to molecules, which have the property of non-superimposability of the mirror image partner, while the term “achiral” refers to molecules, which are superimposable on their mirror image partner.

The term “stereoisomers” refers to compounds, which have identical chemical constitution, but differ with regard to the arrangement of the atoms or groups in space.

“Diastereomer” refers to a stereoisomer with two or more centers of chirality in which the compounds are not mirror images of one another. Diastereomers have different physical properties, e.g. melting points, boiling points, spectral properties, and chemical and biological reactivities. Mixtures of diastereomers may be separated under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McRaw-Hiff Dictionary of Chemical Terms (1984), McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes R and S, are used to denote the absolute configuration of the molecule about its chiral center(s). For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate. The term “tautomer” or “tautomeric form” refers to structural isomers of different energies, which are interconvertible via a low energy barrier. For example, proton tautomers include interconversions via migration of a proton, such as keto-enol and imine-enamine isomerizations.

The term “enone” refers to a α,β-unsaturated carbonyl, that is a type of organic compound consisting of an alkene conjugated to a ketone. The simplest enone is methyl vinyl ketone (butenone) or CH₂═CHCOCH₃. They are electrophilic at both the carbonyl carbon as well as the β-carbon. Depending on conditions, either site is attacked by nucleophiles. Additions to the alkene are called Michael additions and are used in the present invention to covalently modify Cysteine 862 in PI3Kα.

The term “acrylamide” refers to an amide that is derived from acrylic acid and has the general chemical formula CH₂═CHC(O)NH₂. Acrylamides are used in compounds of the present invention and undergo Michael addition with Cysteine 862 in PI3Kα.

The term “PI3K” refers to phosphoinositide 3-kinase.

The term “PI3Kalpha”, “PI3Kα” or “p110a protein” relates to a subunit of PI3K that is encoded by the PI3KCA gene.

The term “irreversible” or “irreversible inhibitor” refers to an inhibitor that is able to be covalently bonded to a PI3 kinase in a substantially non-reversible manner, whereas a reversible inhibitor is able to bind to (but is generally unable to form a covalent bond with) a kinase, and therefore can be dissociated from the PI3 kinase. An irreversible inhibitor will remain substantially bound to a kinase once covalent bond formation has occurred. Methods for identifying if a compound is acting as an irreversible inhibitor are known to one of ordinary skill in the art. Such methods include, but are not limited to, enzyme kinetic analysis of the inhibition profile of the compound with the kinase, the use of mass spectrometry of the protein drug target modified in the presence of the inhibitor compound, the use of X-ray crystallography to solve the complex between the protein drug target and the inhibitor compound, discontinuous exposure, also known as “washout” experiments, as well as other methods known to one of skill in the art.

The term “warhead” or “warhead group” refers to a functional group present on a compound of the present invention wherein that functional group is capable of covalently binding to an amino acid residue (such as cysteine, lysine, histidine, or other residues capable of being covalently modified) present in the binding pocket of the target protein, thereby irreversibly inhibiting the protein. Warhead groups are essential for covalently, and irreversibly, inhibiting the protein.

The term “inhibitor” is defined as a compound that binds to and inhibits PI3 kinase with measurable affinity. In certain embodiments, the inhibitors are characterized by IC₅₀ and/or rate constant for irreversible inactivation (k_{inact}).

The term “CNX-1351” refers to 1-[4-[[2-(1H-indazol-4-yl)-4-(4-morpholinyl)thieno[3,2-d]pyrimidin-6-yl]methyl]-1-piperazinyl]-6-methyl-5-heptene-1,4-dione (CAS 1276105-89-5).

The phrase “pharmaceutically acceptable salt” as used herein, refers to pharmaceutically acceptable organic or inorganic salts of a compound of the invention. If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

The term “protecting group” refers to a substituent that is commonly employed to block or protect a particular functionality during the reaction of other functional groups on the compound. For example, an “amino-protecting group” is a substituent attached to an amino group that blocks or protects the amino functionality in the compound. Suitable amino-protecting groups include acetyl, trifluoroacetyl, tert-butoxycarbonyl (BOC), benzyloxycarbonyl and 9-fluorenylmethylenoxycarbonyl (Fmoc). For a general description of protecting groups and their use, see T. W. Greene, Protective Groups in Organic Synthesis, John Wiley & Sons, New York, 1991.

The terms “compound of this invention” and “compounds of the present invention” and “compounds of formula (I, Ia, II, IIa, III, IIIa, IV, V)” include stereoisomers, geometric isomers, tautomers, solvates, pharmaceutically acceptable salts, and solvates of the salts thereof.

### DETAILED DESCRIPTION

The invention relates to novel triazine-based compounds acting as reversible or irreversible modifiers of PI3 kinase, particularly PI3Kα, and their use as therapeutic agents and chemical probes.

Compounds of the present invention have higher aqueous solubility (>30-fold), higher potency in vitro and in cell (>7-fold), and higher metabolic stability compared to the known inhibitor CNX-1351.

An important aspect of the invention relates to superior reaction parameters in preferred molecules (particularly inhibitors characterized by a warhead that forms an acrylamide moiety) that result in a highly selective target engagement and reduced or negligible off-target reactivity:
i) the on-target reaction of the covalent inhibitors is defined by the inhibitor dissociation constant Kᵢ, which described the reversible equilibrium of enzyme (E) and inhibitor (I) complex formation

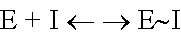

and k_{inact}, which defines the reaction rate of covalent bond formation between inhibitor and enzyme to form the covalent inhibitor-enzyme complex (EI):

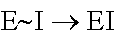

ii) off-target reactions with ubiquitous sulfhydryls (S) and other nucleophiles present in physiologic environments such as cells and body fluids are driven by the intrinsic reactivity of the warhead that is defined by k_{chem} driving the conversion of S and I to form the sulfhydryl-adduct (SI)

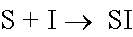

High intrinsic warhead reactivity (high k_{chem} values) leads to the formation of unwanted sulfhydryl-adducts resulting in loss of compound, covalent modification of cellular components, toxicity and anti-compound immune reactions:

As illustrated in Table 1, FIG. 3 and FIG. 4, preferred molecules show a low k_{chem}, low Kᵢ, high k_{inact}, a high k_{inact}/Kᵢ ratio to warrant superior on- versus off target engagement.

A first aspect of the invention relates to a compound of formula (IV), particularly of formula (IVa), or a prodrug, metabolite, tautomer, solvate or pharmaceutically acceptable salt thereof, particularly a tautomer, solvate or pharmaceutically acceptable salt thereof,

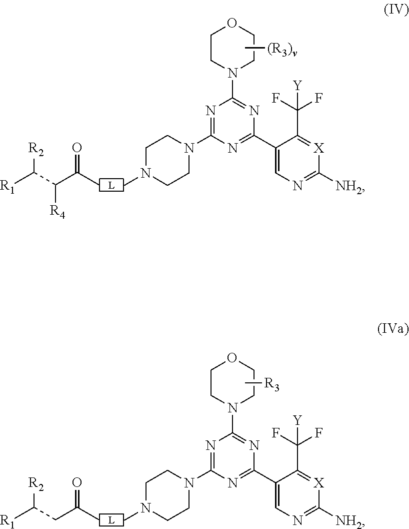

wherein
X is CH or N,
Y is H or F,
R₁ and R₂ are independently of each other selected from H, CH₃, cyclopropyl, —F, —CH₂—F, —CH₂—CH₂—F, —CN,

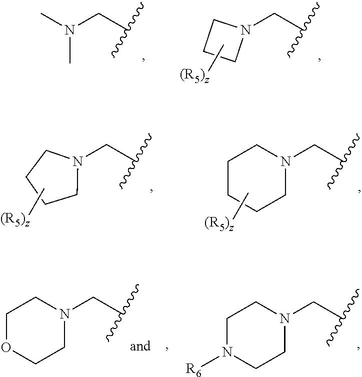

with R₅ being F or CH₃, R₆ being C₁₋₆-alkyl and z being 0, 1 or 2,
R₃ is C₁₋₃-alkyl or two residues R₃ form a bridge —(CH₂)ᵣ— with r being 1, 2 or 3, particularly R₃ is C₁₋₃-alkyl, more particularly CH₃,
v is 0, 1, 2, 3 or 4, particularly 0, 1 or 2, even more particularly 0 or 1,
R₄ is H, F or —CN, particularly H.
L is a linker moiety composed of C, N, O and/or H atoms, wherein the linker moiety L has a length between 3 Å and 15 Å, particularly between 7 Å and 14 Å, more particularly between 10 Å and 13 Å, even more particularly between 10.5 Å and 12.5 Å,
the dashed line represents a carbon-carbon single bond or carbon-carbon double bond, particularly a carbon-carbon double bond.

An inhibitor according to the present invention comprises a multi-cyclic scaffold and a so-called warhead that is connected to the scaffold via a linker. The scaffold comprises a triazine moiety that is substituted by three heterocycles, namely a morpholinyl, a piperazinyl and a pyridinyl or pyrimidinyl moiety. The pyridinyl or pyrimidinyl moiety is substituted by a fluorinated methyl and an amine moiety. The morpholinyl moiety is optionally substituted. The piperazinyl is bound to the linker moiety L.

The molecules present certain reactive functional groups so called “warheads.” As used herein, the term “warhead” or “warhead group” refers to a functional group present on a compound of the present invention wherein that functional group is capable of covalently binding to an amino acid residue (such as cysteine, lysine, histidine, or other residues capable of being covalently modified) present in the binding pocket of the target protein, thereby irreversibly inhibiting the protein.

The inhibitors according to the present invention may bind covalently to Cys862 of PI3Kα. Such inhibitors are characterized by a carbon-carbon double bond in the warhead. The stability of the bond may be modulated by substituents R₁, R₂ and R₄ to achieve inhibitors that form a stable covalent bond or a reversible covalent bond (see below).

Alternatively, an inhibitor of the present invention may reversibly inhibit PI3Kα without forming a covalent bond to a moiety of PI3Kα. Such inhibitors are characterized by a carbon-carbon single bond in the warhead.

If the inhibitor binds to PI3Kα, the warhead has to be in close proximity to Cys862 to obtain an inhibitory effect. This is achieved by a linker moiety that has a length between 3 Å and 15 Å, particularly between 7 Å and 14 Å, more particularly between 10 Å and 13 Å, even more particularly between 10.5 Å and 12.5 Å.

In certain embodiments, the linker moiety L has a length that corresponds to a linear alkyl chain of 4 to 7 C atoms.

A good stability and solubility may be achieved if the linker is composed of C, N, O and/or H atoms. Such linker may also contribute to the tolerance of the inhibitor in a patient.

The compounds disclosed herein show a good PI3 kinase inhibition, particularly a good PI3Kα inhibition, in combination with a good stability.

In certain embodiments, L consists of 2 to 5 moieties independently of each other selected from C₁₋₅-alkyl, —CO—, —NH—, —N(CH₃)—, —O—, phenyl,

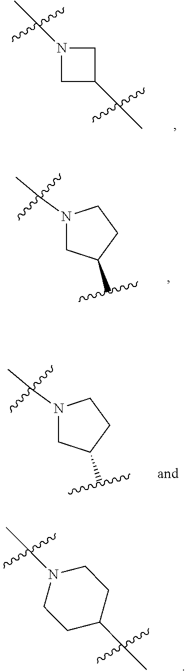

In certain embodiments, L is a moiety selected from
—U-L₁-W—,
-C₁₋₂-alkyl-CO-L₂-(CH₂)ₚ—W—,

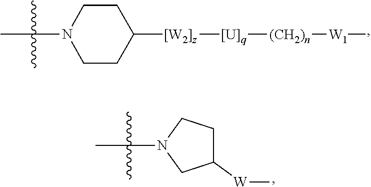

wherein
U is —CH₂— or —NH— or —N(CH₃)— or —O— or phenyl
L₁ is C₁-C₄-alkyl,
L₂ is azetidine, pyrrolidine or piperidine,
W, W₁ and W₂ are independently of each other —CO— or —CH₂—,
n is 0, 1, 2 or 3,
p, q and z are independently of each other 0 or 1.

In certain embodiments, L is a moiety selected from
—U-L₁-W—,

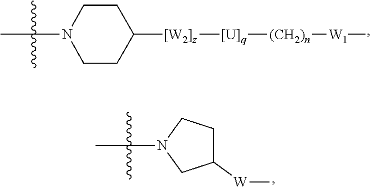

wherein
U is —CH₂— or —NH— or —N(CH₃)— or —O— or phenyl, particularly —NH— or —N(CH₃)—, more particularly —NH—,
L₁ is C₁-C₄-alkyl,
L₂ is azetidine, pyrrolidine or piperidine,
W, W₁ and W₂ are independently of each other —CO— or —CH₂—,
n is 0, 1, 2 or 3,
p, q and z are independently of each other 0 or 1.

As described above, superior reaction parameters were observed particularly for compounds that are characterized by a warhead that forms an acrylamide-based moiety. This is achieved when the linker starts with a nitrogen.

In certain embodiments, L is a moiety selected from
—U-L₁-W—,

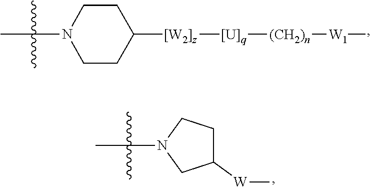

wherein
U is —NH— or —N(CH₃)—, particularly —NH—,
L₁ is C₁-C₄-alkyl,
L₂ is azetidine, pyrrolidine or piperidine,
W, W₁ and W₂ are independently of each other —CO— or —CH₂—,
n is 0, 1, 2 or 3,
p, q and z are independently of each other 0 or 1.

In certain embodiments, L is a moiety

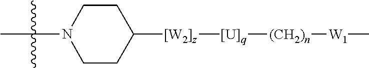

with W₂, z, U, q, n and W₁ as defined above.

In certain embodiments, L is a moiety

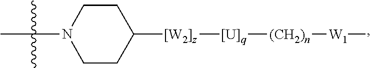

wherein
W₂ is —CH₂—,
z is 0 or 1, particularly 1,
U is —O—,
q is 0 or 1, particularly 1,
n is 0, 1, 2, or 3, particularly 1, 2 or 3, more particularly 1 or 2, even more particularly 1, and
W₁ is —CO—.

As described above, the reactivity and affinity of the warhead may be modified by substituents R₁, R₂ and R₄.

In certain embodiments, R₁ and R₂ are independently of each other selected from H, CH₃,

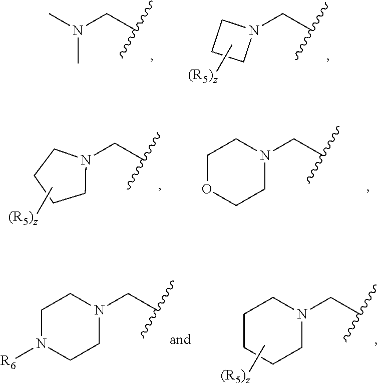

particularly from H, CH₃,

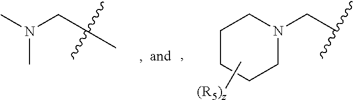

with R₅ being F or CH₃, R₆ being C₁₋₆-alkyl, particularly C₁₋₃-alkyl, more particularly methyl, and z being 0, 1 or 2.

In certain embodiments, R₁ is selected from H, CH₃, —F, —CH₂—F, —CH₂—CH₂—F, —CN,

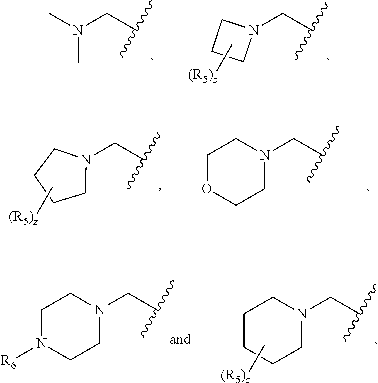

particularly from H, CH₃,

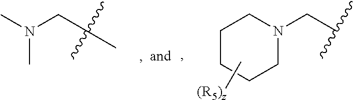

with R₅ being F or CH₃, R₆ being C₁₋₆-alkyl, particularly C₁₋₃-alkyl, more particularly methyl, and z being 0, 1 or 2, and R₂ is H or CH₃, and R₄ is H.

In certain embodiments, R₁ is selected from H, CH₃,

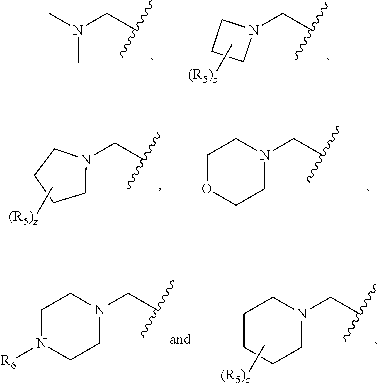

particularly from H, CH₃,

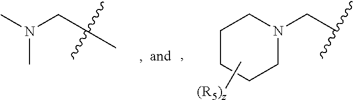

with R₅ being F or CH₃, R₆ being C₁₋₆-alkyl, particularly C₁₋₃-alkyl, more particularly methyl, and z being 0, 1 or 2, and R₂ is H or CH₃, and R₄ is H.

In certain embodiments, R¹ and R² are H. In certain embodiments, R₄ is H.

Reversible covalently binding inhibitors may be achieved by modifying the warhead with cyclopropyl and —CN.

In certain embodiments, R₂ is cyclopropyl and R₄ is —CN.

In certain embodiments, the compound is a compound of formula (IV), wherein the dashed line represents a carbon-carbon double bond, R₂ is cyclopropyl and R₄ is —CN.

In certain embodiments, the compound is a compound of formula (IV), wherein the dashed line represents a carbon-carbon double bond, R₁ is H, R₂ is cyclopropyl and R₄ is —CN.

In certain embodiments, the compound is a compound of formula (IV), wherein the dashed line represents a carbon-carbon double bond, R₁ is H, R₂ is cyclopropyl, R₄ is —CN and Y is H.

In certain embodiments, the compound is a compound of formula (IV), wherein the dashed line represents a carbon-carbon double bond, R₁ is H, R₂ is cyclopropyl, R₃ is methyl, v is 0 or 1, particularly v is 1, R₄ is —CN and Y is H.

In certain embodiments, the compound is a compound of formula (IV), wherein the dashed line represents a carbon-carbon double bond, R₂ is cyclopropyl and R₄ is —CN, and L is a moiety

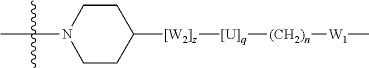

with W₂, z, U, q, n and W₁ as defined above, particularly
W₂ is —CH₂—,
z is 0 or 1, particularly 1,
U is —O—,
q is 0 or 1, particularly 1,
n is 0, 1, 2, or 3, particularly 1, 2 or 3, more particularly 1 or 2, even more particularly 1, and
W₁ is —CO—.

In certain embodiments, the compound is a compound of formula (IV), wherein the dashed line represents a carbon-carbon double bond, R₁ is H, R₂ is cyclopropyl and R₄ is —CN, and L is a moiety

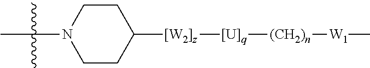

with W₂, z, U, q, n and Wi as defined above, particularly
W₂ is —CH₂—,
z is 0 or 1, particularly 1,
U is —O—,
q is 0 or 1, particularly 1,
n is 0, 1, 2, or 3, particularly 1, 2 or 3, more particularly 1 or 2, even more particularly 1, and
W₁ is —CO—.

In certain embodiments, the compound is a compound of formula (IV), wherein the dashed line represents a carbon-carbon double bond, R₁ is H, R₂ is cyclopropyl, R₄ is —CN and Y is H, and L is a moiety

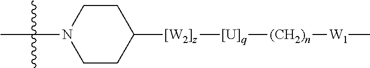

with W₂, z, U, q, n and W₁ as defined above, particularly
W₂ is —CH₂—,
z is 0 or 1, particularly 1,
U is —O—,
q is 0 or 1, particularly 1,
n is 0, 1, 2, or 3, particularly 1, 2 or 3, more particularly 1 or 2, even more particularly 1, and
W₁ is —CO—.

In certain embodiments, the compound is a compound of formula (IV), wherein the dashed line represents a carbon-carbon double bond, R₁ is H, R₂ is cyclopropyl, R₃ is methyl, v is 0 or 1, particularly v is 1, R₄ is —CN and Y is H, and L is a moiety

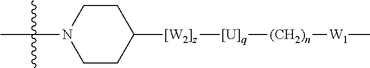

with W₂, z, U, q, n and W₁ as defined above, particularly
W₂ is —CH₂—,
z is 0 or 1, particularly 1,
U is —O—,
q is 0 or 1, particularly 1,
n is 0, 1, 2, or 3, particularly 1, 2 or 3, more particularly 1 or 2, even more particularly 1, and
W₁ is —CO—.

In certain embodiments, the compound is a compound of formula (Ia), particularly of formula (I),

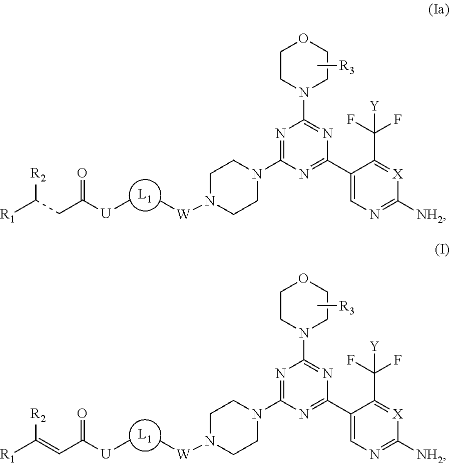

wherein
X is CH or N,
Y is H or F,
W is —CO— or —CH₂—,
U is —CH₂— or —NH— or —N(CH₃)—.
R₁ and R₂ are independently of each other selected from H, CH₃,

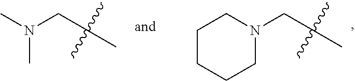

particularly R₁ and R₂ are independently of each other H or —CH₃,
L₁ is C₁-C₄-alkyl,
R₃ is H or —CH₃,
the dashed line represents a carbon-carbon single bond or carbon-carbon double bond, particularly a carbon-carbon double bond.

In certain embodiments, R¹ and R² in a compound according to formula (Ia) or (I) are H.

In certain embodiments, W is —CO— and U is —CH₂— in a compound according to formula (Ia) or (I).

In certain embodiments, the compound is a compound of formula (IIa), particularly of formula (II),

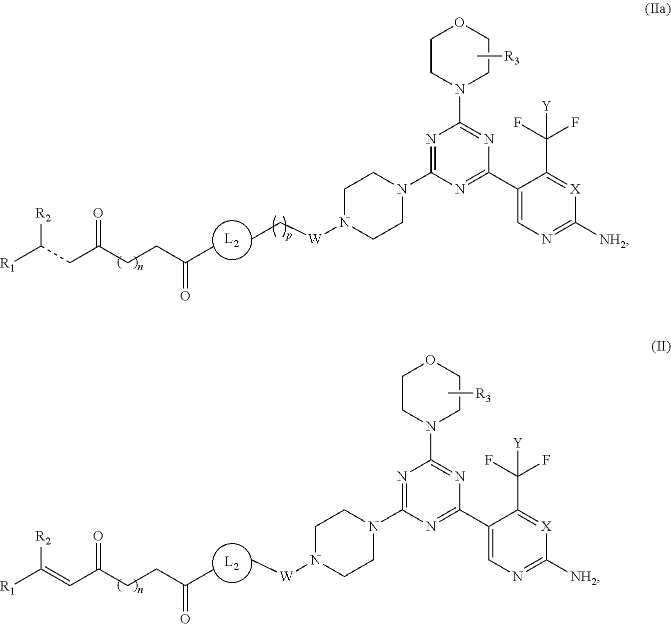

wherein
X is CH or N,
Y is H or F,
W is CO or CH₂,
L₂ is azetidine or pyrrolidine or piperidine, particularly

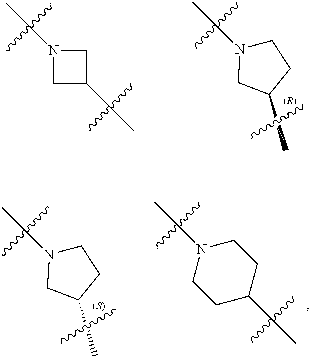

R₁ and R₂ are independently of each other selected from H, CH₃,

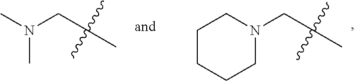

particularly R₁ and R₂ are independently of each other selected from H and CH₃.
R₃ is H or CH₃.
n is equal to 1 or 2,
p is equal to 0 or 1,
   the dashed line represents a carbon-carbon single bond or carbon-carbon double bond, particularly a carbon-carbon double bond.

In certain embodiments, the compound is a compound of formula (IIIa), particularly of formula (III),

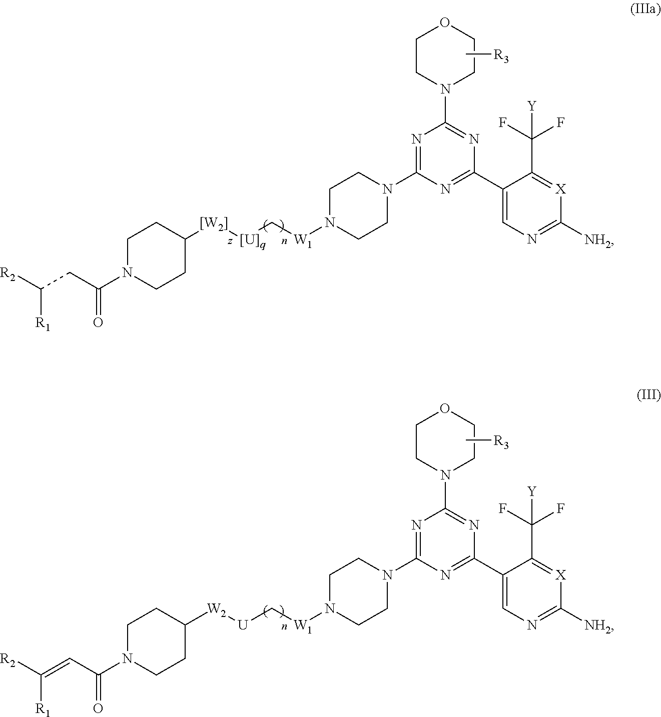

wherein
X is CH or N,
Y is H or F,
W₁ and W₂ are independently of each other —CO— or —CH₂—,
U is —CH₂— or —NH— or —N(CH₃)— or —O— or phenyl,
R₁ and R₂ are independently of each other selected form H, CH₃,

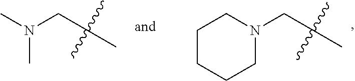

R₃ is H or CH₃,
n is equal to 0 or 1 or 2 or 3,
z is equal to 0 or 1
q is equal to 0 or 1,
the dashed line represents a carbon-carbon single bond or carbon-carbon double bond, particularly a carbon-carbon double bond.

In certain embodiments, R¹ and R² in a compound according to formula (IIIa) or (III) are H.

In certain embodiments, W₁ is —CO— and U is —O— or —N(CH₃)— in a compound according to formula (IIIa) or (III).

A second aspect of the invention relates to a compound according to the first aspect of the invention for use in the treatment of a disease.

Another aspect of the invention relates to a compound according to the first aspect of the invention for use in the treatment of a disease, wherein the disease caused by an activating mutation of the PI3KCA gene or activation of a class I PI3K, in particular PI3Kα. The activation of the class I PI3K, in particular PI3Kα, can occur by cell surface receptors, upstream over-expressed or mutated up-stream activators or an activating mutation of the PI3KCA gene or PI3K-interacting and regulatory proteins, including the gene products of PIK3R₁, PIK3R₁, PIK3R1.

A third aspect of the invention relates to a compound according to the first aspect of the invention for use in the treatment of tumor disease, overgrowth syndrome, neurological disease, immunological disease.

In certain embodiments, the tumor is a solid tumor and/or the tumor disease is selected from lymphoma and leukemia.

In certain embodiments, the compound according to the first aspect of the invention is for use in the treatment of a proliferative disease; any benign or malignant tumor; a tumors emerging from sarcoma; lung; bronchus; prostate; breast; pancreas; gastrointestinal cancer; colon; rectum; colon carcinoma; colorectal adenoma; thyroid; liver; intrahepatic bile duct; hepatocellular; adrenal gland; stomach; gastric; glioma; glioblastoma; endometrial; melanoma; kidney; renal pelvis; urinary bladder; uterine corpus; uterine cervix; vagina; ovary; multiple myeloma; esophagus; the treatment of leukaemia; acute myelogenous leukemia; chronic myelogenous leukemia; lymphocytic leukemia; myeloid leukemia; brain; a carcinoma of the brain; oral cavity and pharynx; larynx; small intestine; non-Hodgkin lymphoma; melanoma; villous colon adenoma; a neoplasia; a neoplasia of epithelial character; lymphomas; the treatment of mammary carcinomas; basal cell carcinomas; squamous cell carcinomas; actinic keratosis; tumor diseases, including solid tumors; a tumor of the neck or head; polycythemia vera; essential thrombocythemia; myelofibrosis with myeloid metaplasia; and Waldenström's macroglobulinemia; Cowden's disease and multiple hamartoma syndrome including sporadic cancers such as breast, thyroid, uterine cancers, and other cancers emerging in patients with these syndromes; PIK3CA-related overgrowth spectrum (PROS)-related disorders, including Fibroadipose hyperplasia (also called fibroadipose overgrowth), CLOVES syndrome, Megalencephaly-capillary malformation syndrome (MCAP syndrome), Hemihyperplasia-multiple lipomatosis syndrome (HHML syndrome), Hemimegalencephaly, and Facial and infiltrating lipomatosis in other organs; general vascular and lymphatic malformations, and tumor neovascularization; ocular neovascularization and macular degeneration (AMD), proliferative and diabetic retinopathy (PDR), and retinopathy of prematurity (ROP)”.

A fourth aspect of the invention relates to an intermediate of formula (V),

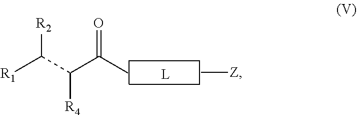

wherein
R₁, R₂, R₄ and L are defined as described above,
Z is —OH, Br, COOH or NH₂.

In certain embodiments, the intermediate is a compound of formula (Va), and/or of formula (Vb), and/or of formula (Vc),

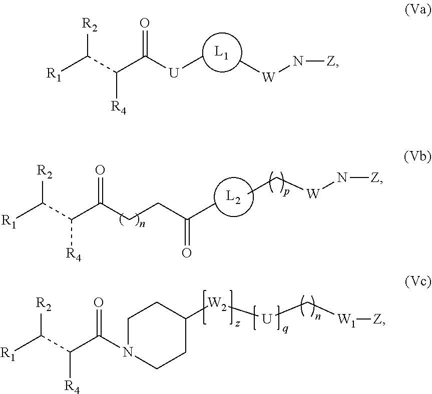

wherein R₁, R₂, R₄, U, L₁, W, n, L₂, p, W₂, q, W₁ and Z are defined as described above. Reference is made to the embodiments of the first aspect of the invention.

The invention also relates to such PI3K-targeting compounds as chemotherapeutic agents with anti-cancer activity, pharmaceutical formulations thereof, which are potentially useful in treatment of disease, conditions and/or disorders modulated by augmented cell division, growth, migration, adhesion and metastasis. The compounds may inhibit tumor growth in mammals, and may be useful for treating human cancer patients.

Additionally, to cancer treatment, the present invention also relates to the treatment of pathogenic cellular states caused by cell over-activation using such compounds. Molecules targeting PI3K can be used to treat a variety of hyper-proliferative diseases for the treatment or prevention of a disease or condition modulated by PI3Ks.

The invention also relates to such PI3K-targeting compounds as chemical probes to dissect the role(s) of PI3K isoforms in cancer and metabolism.

In addition, the invention relates to methods of using such compounds for in vitro, in situ, and in vivo diagnostic procedures or treatment of mammalian cells, organisms, or associated pathological conditions, or production processes.

Another aspect of the invention includes methods of preparing, methods of separating, and methods of purifying compounds of formula (I, II, III, IV, V), as defined hereinbefore including intermediates, prodrugs and salts of said compounds.

Another aspect of the invention includes novel intermediates useful for preparing compounds of formula (I, II, III, IV, V) as defined hereinbefore.

Another aspect of the invention includes the novel, improved properties of compounds of formula (I, II, III) with respect to CNX-1351. These properties include, but are not limited to, in vitro and cellular potency, metabolic stability, solubility and drug-likeness properties.

Further aspects and embodiments of the present invention will be become apparent as this description continues.

The cysteine residue of PI3Kα targeted for covalent modification by irreversible inhibitors of the present invention is the non-conserved Cys862.

### Medical treatment, Dosage Forms and Salts

Similarly, within the scope of the present invention is a method for treating tumor disease, overgrowth syndrome, neurological disease disorder and/or immunological disease disorder in a patient in need thereof, comprising administering to the patient a compound according to the above description.

Similarly, a dosage form for the prevention or treatment of tumor disease, overgrowth syndrome, neurological disease disorder and/or immunological disease disorder is provided, comprising compound according to any of the above aspects or embodiments of the invention.

The skilled person is aware that any specifically mentioned drug compound mentioned herein may be present as a pharmaceutically acceptable salt of said drug. Pharmaceutically acceptable salts comprise the ionized drug and an oppositely charged counterion. Non-limiting examples of pharmaceutically acceptable anionic salt forms include acetate, benzoate, besylate, bitatrate, bromide, carbonate, chloride, citrate, edetate, edisylate, embonate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate, phosphate, diphosphate, salicylate, disalicylate, stearate, succinate, sulfate, tartrate, tosylate, triethiodide and valerate. Non-limiting examples of pharmaceutically acceptable cationic salt forms include aluminium, benzathine, calcium, ethylene diamine, lysine, magnesium, meglumine, potassium, procaine, sodium, tromethamine and zinc.

Dosage forms may be for enteral administration, such as nasal, buccal, rectal, transdermal or oral administration, or as an inhalation form or suppository. Alternatively, parenteral administration may be used, such as subcutaneous, intravenous, intrahepatic or intramuscular injection forms. Optionally, a pharmaceutically acceptable carrier and/or excipient may be present.

Topical administration is also within the scope of the advantageous uses of the invention. The skilled artisan is aware of a broad range of possible recipes for providing topical formulations, as exemplified by the content of Benson and Watkinson (Eds.), Topical and Transdermal Drug Delivery: Principles and Practice (1st Edition, Wiley 2011, ISBN-13: 978-0470450291); and Guy and Handcraft: Transdermal Drug Delivery Systems: Revised and Expanded (2^{nd} Ed., CRC Press 2002, ISBN-13: 978-0824708610); Osborne and Amann (Eds.): Topical Drug Delivery Formulations (1^{st} Ed. CRC Press 1989; ISBN-13: 978-0824781835).

### Pharmaceutical Compositions and Administration

Another aspect of the invention relates to a pharmaceutical composition comprising a compound of the present invention, or a pharmaceutically acceptable salt thereof, and a pharmaceutically acceptable carrier. In further embodiments, the composition comprises at least two pharmaceutically acceptable carriers, such as those described herein.

In certain embodiments of the invention, the compound of the present invention is typically formulated into pharmaceutical dosage forms to provide an easily controllable dosage of the drug and to give the patient an elegant and easily handleable product.

In embodiments of the invention relating to topical uses of the compounds of the invention, the pharmaceutical composition is formulated in a way that is suitable for topical administration such as aqueous solutions, suspensions, ointments, creams, gels or sprayable formulations, e.g., for delivery by aerosol or the like, comprising the active ingredient together with one or more of solubilizers, stabilizers, tonicity enhancing agents, buffers and preservatives that are known to those skilled in the art.

The pharmaceutical composition can be formulated for enteral administration, particularly oral administration or rectal administration. In addition, the pharmaceutical compositions of the present invention can be made up in a solid form (including without limitation capsules, tablets, pills, granules, powders or suppositories), or in a liquid form (including without limitation solutions, suspensions or emulsions).

The pharmaceutical composition can be formulated for parenteral administration, for example by i.v. infusion, intradermal, subcutaneous or intramuscular administration.

The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

In certain embodiments, the pharmaceutical composition of the present invention can be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The pharmaceutical compositions of the present invention can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

### Method of Manufacture and Method of Treatment According to the Invention

The invention further encompasses, as an additional aspect, the use of a compound according to the first aspect of the invention as identified herein, or its pharmaceutically acceptable salt, as specified in detail above, for use in a method of manufacture of a medicament for the treatment or prevention of a condition selected from tumor disease, overgrowth syndrome, neurological disease disorder and/or immunological disease disorder.

Similarly, the invention encompasses methods of treatment of a patient having been diagnosed with a disease associated with tumor disease, overgrowth syndrome, neurological disease disorder and/or immunological disease disorder. This method entails administering to the patient an effective amount of compound as identified herein (SPECIFY), or its pharmaceutically acceptable salt, as specified in detail herein.

Wherever alternatives for single separable features such as, for example, an isotype protein or coding sequence, ligand type or medical indication are laid out herein as “embodiments”, it is to be understood that such alternatives may be combined freely to form discrete embodiments of the invention disclosed herein. Thus, any of the alternative embodiments for a detectable label may be combined with any of the alternative embodiments of ligand and these combinations may be combined with any medical indication or diagnostic method mentioned herein.

The invention is further illustrated by the following examples and figures, from which further embodiments and advantages can be drawn. These examples are meant to illustrate the invention but not to limit its scope.

In certain embodiments, the compound according to the first aspect of the invention is selected from compounds 1 to 117 as shown in the following table.

In certain embodiments, the compound according to the first aspect of the invention is selected from compounds 1 to 119 as shown in the following table.

In certain embodiments, the compound according to the first aspect of the invention is selected from compounds 118 and 119 as shown in the following table.

In certain embodiments, the compound according to the first aspect of the invention is compound 118 as shown in the following table.

| | |
|---|---|
| 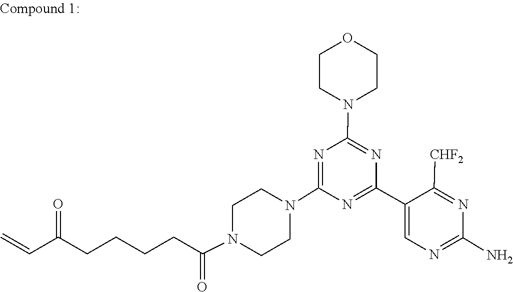 [SMILES: C=CC(=O)CCCCC(=O)N1CCN(c2nc(-c3cnc(N)nc3C(F)F)nc(N3CCOCC3)n2)CC1] | 1-(4-(4-(2-amino-4-(difluoromethyl)pyrimidin- 5-yl)-6-morpholino-1,3,5-triazin-2-yl)piperazin- 1-yl)oct-7-ene-1,6-dione |
| 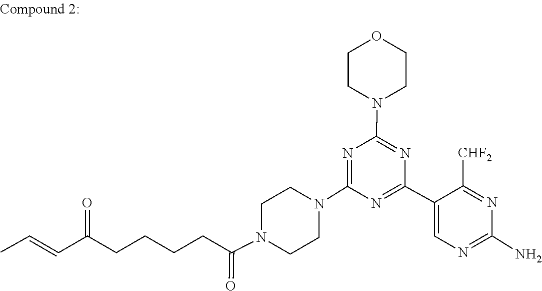 [SMILES: C/C=C/C(=O)CCCCC(=O)N1CCN(c2nc(-c3cnc(N)nc3C(F)F)nc(N3CCOCC3)n2)CC1] | (E)-1-(4-(4-(2-amino-4- (difluoromethyl)pyrimidin-5-yl)-6-morpholino- 1,3,5-triazin-2-yl)piperazin-1-yl)non-7-ene-1,6- dione |
| 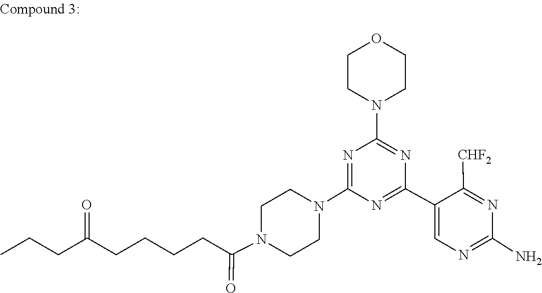 [SMILES: CCCC(=O)CCCCC(=O)N1CCN(c2nc(-c3cnc(N)nc3C(F)F)nc(N3CCOCC3)n2)CC1] | 1-(4-(4-(2-amino-4-(difluoromethyl)pyrimidin- 5-yl)-6-morpholino-1,3,5-triazin-2-yl)piperazin- 1-yl)nonane-1,6-dione |
| 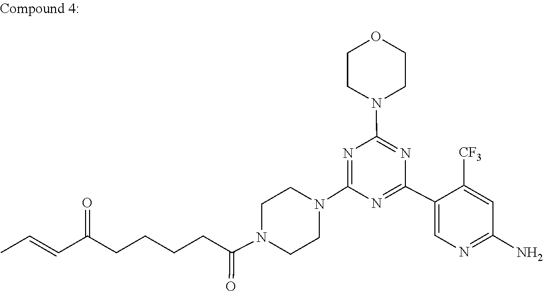 | (E)-1-(4-(4-(6-amino-4-(trifluoromethyl)pyridin- 3-yl)-6-morpholino-1,3,5-triazin-2-yl)piperazin- 1-yl)non-7-ene-1,6-dione |
| 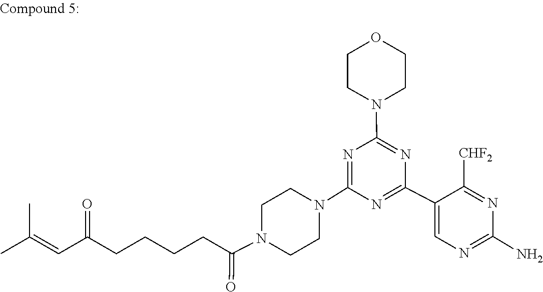 | 1-(4-(4-(2-amino-4-(difluoromethyl)pyrimidin- 5-yl)-6-morpholino-1,3,5-triazin-2-yl)piperazin- 1-yl)-8-methylnon-7-ene-1,6-dione |
| 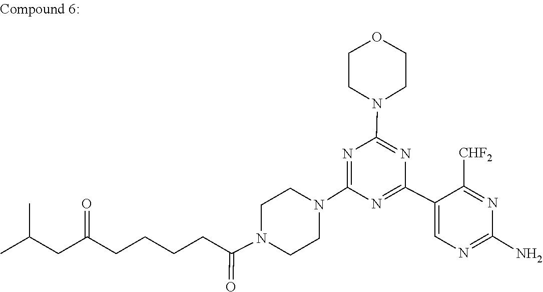 | 1-(4-(4-(2-amino-4-(difluoromethyl)pyrimidin- 5-yl)-6-morpholino-1,3,5-triazin-2-yl)piperazin- 1-yl)-8-methylnonane-1,6-dione |
| 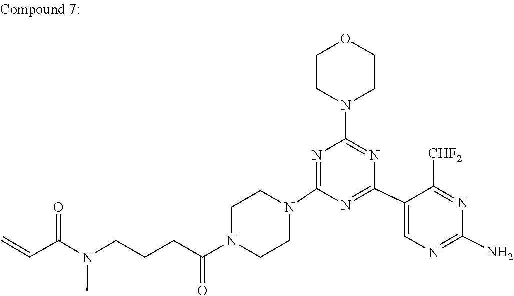 [SMILES: C=CC(=O)N(C)CCCC(=O)N1CCN(c2nc(-c3cnc(N)nc3C(F)F)nc(N3CCOCC3)n2)CC1] | N-(4-(4-(4-(2-amino-4- (difluoromethyl)pyrimidin-5-yl)-6-morpholino- 1,3,5-triazin-2-yl)piperazin-1-yl)-4-oxobutyl)-N- methylacrylamide |
| 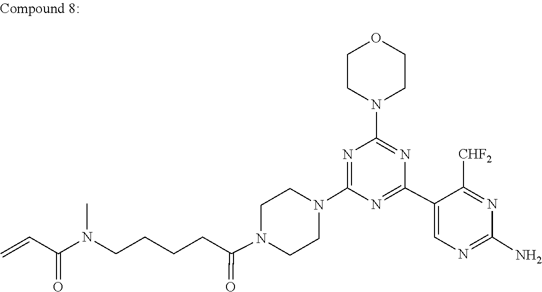 [SMILES: C=CC(=O)N(C)CCCCC(=O)N1CCN(c2nc(-c3cnc(N)nc3C(F)F)nc(N3CCOCC3)n2)CC1] | N-(5-(4-(4-(2-amino-4- (difluoromethyl)pyrimidin-5-yl)-6-morpholino- 1,3,5-triazin-2-yl)piperazin-1-yl)-5-oxopentyl)- N-methylacrylamide |
| 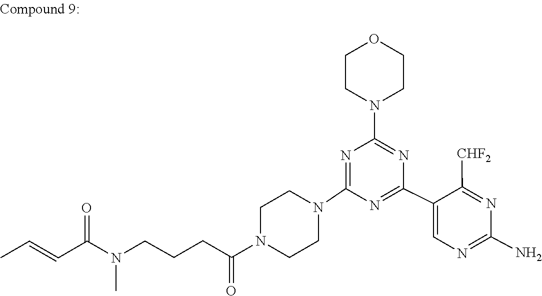 [SMILES: C/C=C/C(=O)N(C)CCCC(=O)N1CCN(c2nc(-c3cnc(N)nc3C(F)F)nc(N3CCOCC3)n2)CC1] | (E)-N-(4-(4-(4-(2-amino-4- (difluoromethyl)pyrimidin-5-yl)-6-morpholino- 1,3,5-triazin-2-yl)piperazin-1-yl)-4-oxobutyl)-N- methylbut-2-enamide |
| 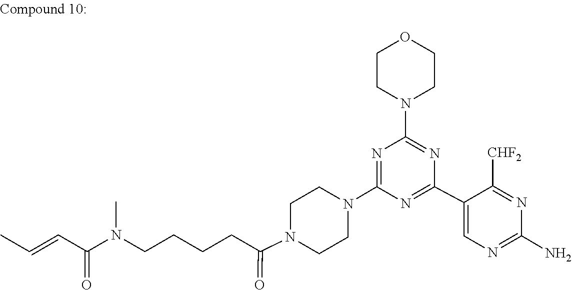 [SMILES: C/C=C/C(=O)N(C)CCCCC(=O)N1CCN(c2nc(-c3cnc(N)nc3C(F)F)nc(N3CCOCC3)n2)CC1] | (E)-N-(5-(4-(4-(2-amino-4- (difluoromethyl)pyrimidin-5-yl)-6-morpholino- 1,3,5-triazin-2-yl)piperazin-1-yl)-5-oxopentyl)- N-methylbut-2-enamide |
| 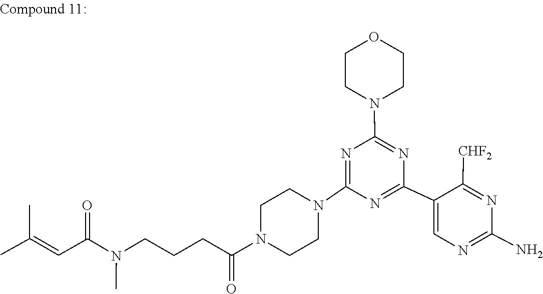 [SMILES: CC(C)=CC(=O)N(C)CCCC(=O)N1CCN(c2nc(-c3cnc(N)nc3C(F)F)nc(N3CCOCC3)n2)CC1] | N-(4-(4-(4-(2-amino-4- (difluoromethyl)pyrimidin-5-yl)-6-morpholino- 1,3,5-triazin-2-yl)piperazin-1-yl)-4-oxobutyl)- N,3-dimethylbut-2-enamide |
| 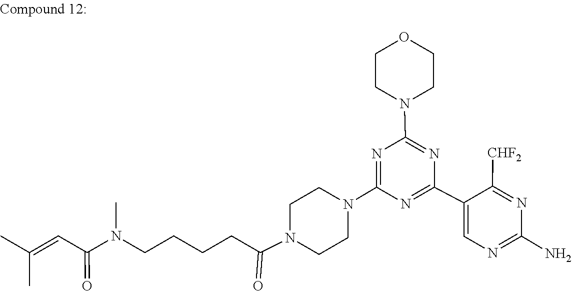 [SMILES: CC(C)=CC(=O)N(C)CCCCC(=O)N1CCN(c2nc(-c3cnc(N)nc3C(F)F)nc(N3CCOCC3)n2)CC1] | N-(5-(4-(4-(2-amino-4- (difluoromethyl)pyrimidin-5-yl)-6-morpholino- 1,3,5-triazin-2-yl)piperazin-1-yl)-5-oxopentyl)- N,3-dimethylbut-2-enamide |
| 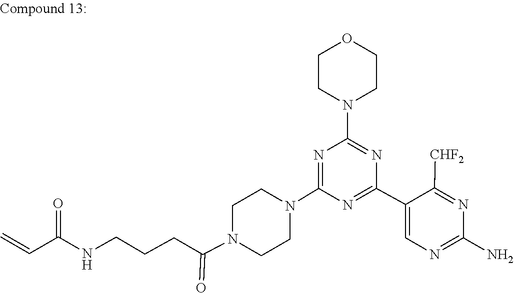 [SMILES: C=CC(=O)NCCCC(=O)N1CCN(c2nc(-c3cnc(N)nc3C(F)F)nc(N3CCOCC3)n2)CC1] | N-(4-(4-(4-(2-amino-4- (difluoromethyl)pyrimidin-5-yl)-6-morpholino- 1,3,5-triazin-2-yl)piperazin-1-yl)-4- oxobutyl)acrylamide |
| 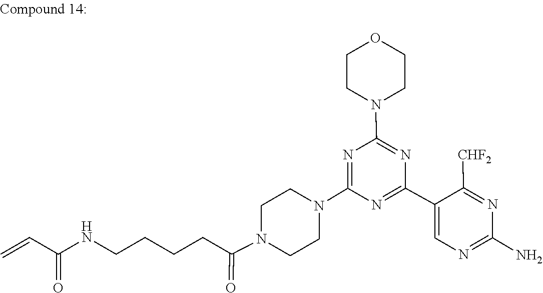 [SMILES: C=CC(=O)NCCCCC(=O)N1CCN(c2nc(-c3cnc(N)nc3C(F)F)nc(N3CCOCC3)n2)CC1] | N-(5-(4-(4-(2-amino-4- (difluoromethyl)pyrimidin-5-yl)-6-morpholino- 1,3,5-triazin-2-yl)piperazin-1-yl)-5- oxopentyl)acrylamide |
| 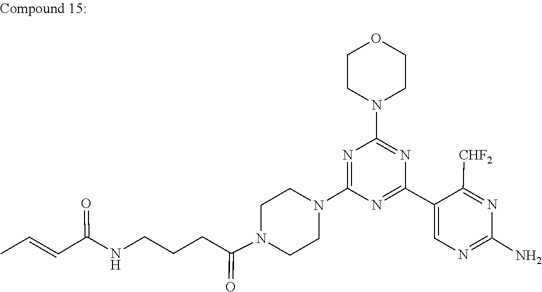 [SMILES: C/C=C/C(=O)NCCCC(=O)N1CCN(c2nc(-c3cnc(N)nc3C(F)F)nc(N3CCOCC3)n2)CC1] | (E)-N-(4-(4-(4-(2-amino-4- (difluoromethyl)pyrimidin-5-yl)-6-morpholino- 1,3,5-triazin-2-yl)piperazin-1-yl)-4- oxobutyl)but-2-enamide |
| 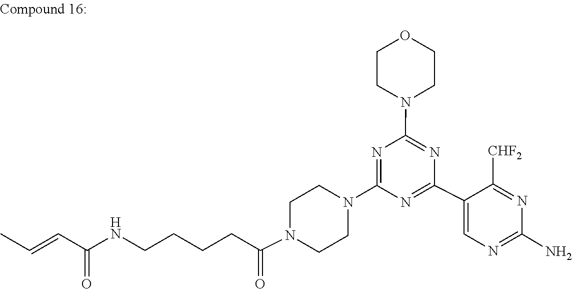 | (E)-N-(5-(4-(4-(2-amino-4- (difluoromethyl)pyrimidin-5-yl)-6-morpholino- 1,3,5-triazin-2-yl)piperazin-1-yl)-5- oxopentyl)but-2-enamide |
| 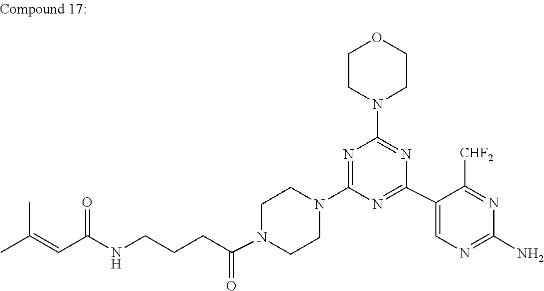 | N-(4-(4-(4-(2-amino-4- (difluoromethyl)pyrimidin-5-yl)-6-morpholino- 1,3,5-triazin-2-yl)piperazin-1-yl)-4-oxobutyl)-3- methylbut-2-enamide |
| 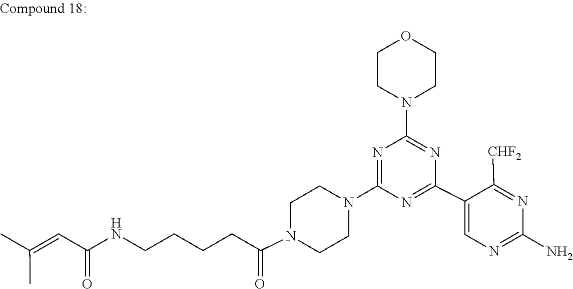 | N-(5-(4-(4-(2-amino-4- (difluoromethyl)pyrimidin-5-yl)-6-morpholino- 1,3,5-triazin-2-yl)piperazin-1-yl)-5-oxopentyl)- 3-methylbut-2-enamide |
| 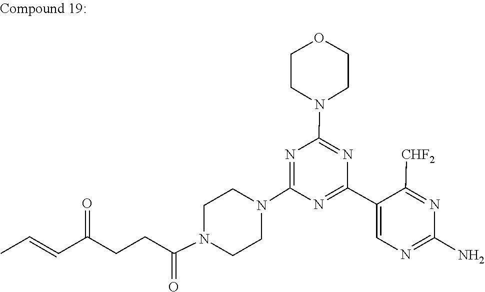 | (E)-1-(4-(4-(2-amino-4- (difluoromethyl)pyrimidin-5-yl)-6-morpholino- 1,3,5-triazin-2-yl)piperazin-1-yl)hept-5-ene- 1,4-dione |
| 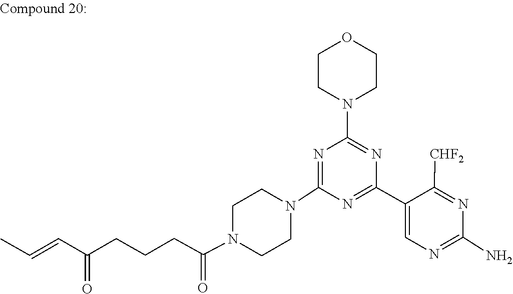 [SMILES: C/C=C/C(=O)CCCC(=O)N1CCN(c2nc(-c3cnc(N)nc3C(F)F)nc(N3CCOCC3)n2)CC1] | (E)-1-(4-(4-(2-amino-4- (difluoromethyl)pyrimidin-5-yl)-6-morpholino- 1,3,5-triazin-2-yl)piperazin-1-yl)oct-6-ene-1,5- dione |
| 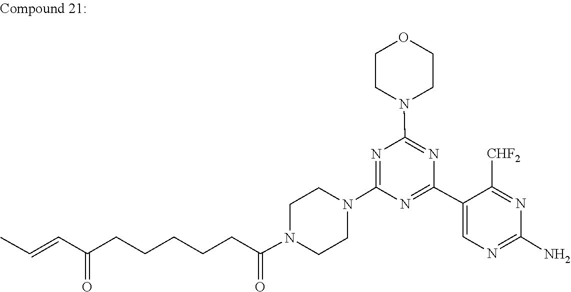 [SMILES: C/C=C/C(=O)CCCCCC(=O)N1CCN(c2nc(-c3cnc(N)nc3C(F)F)nc(N3CCOCC3)n2)CC1] | (E)-1-(4-(4-(2-amino-4- (difluoromethyl)pyrimidin-5-yl)-6-morpholino- 1,3,5-triazin-2-yl)piperazin-1-yl)dec-8-ene-1,7- dione |
| 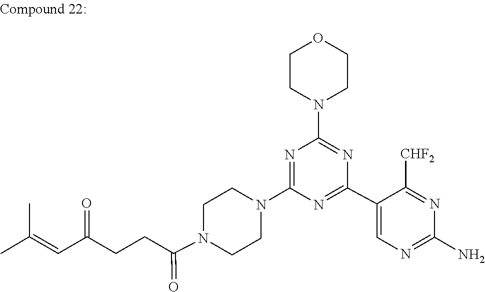 [SMILES: CC(C)=CC(=O)CCC(=O)N1CCN(c2nc(-c3cnc(N)nc3C(F)F)nc(N3CCOCC3)n2)CC1] | 1-(4-(4-(2-amino-4-(difluoromethyl)pyrimidin- 5-yl)-6-morpholino-1,3,5-triazin-2-yl)piperazin- 1-yl)-6-methylhept-5-ene-1,4-dione |
| 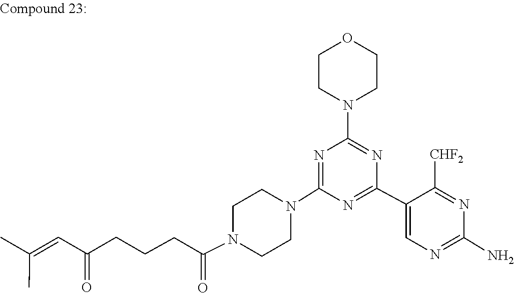 [SMILES: CC(C)=CC(=O)CCCC(=O)N1CCN(c2nc(-c3cnc(N)nc3C(F)F)nc(N3CCOCC3)n2)CC1] | 1-(4-(4-(2-amino-4-(difluoromethyl)pyrimidin- 5-yl)-6-morpholino-1,3,5-triazin-2-yl)piperazin- 1-yl)-7-methyloct-6-ene-1,5-dione |
| 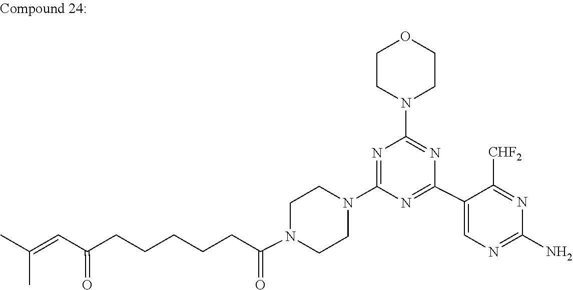 | 1-(4-(4-(2-amino-4-(difluoromethyl)pyrimidin- 5-yl)-6-morpholino-1,3,5-triazin-2-yl)piperazin- 1-yl)-9-methyldec-8-ene-1,7-dione |
| 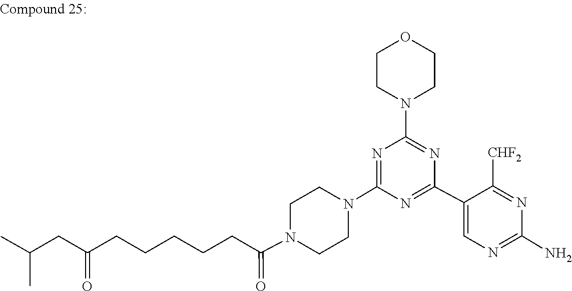 | 1-(4-(4-(2-amino-4-(difluoromethyl)pyrimidin- 5-yl)-6-morpholino-1,3,5-triazin-2-yl)piperazin- 1-yl)-9-methyldecane-1,7-dione |
| 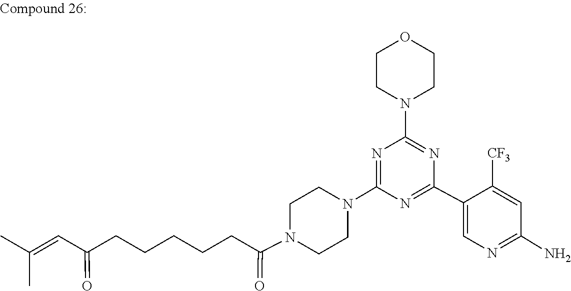 [SMILES: CC(C)=CC(=O)CCCCCC(=O)N1CCN(c2nc(-c3cnc(N)cc3C(F)(F)F)nc(N3CCOCC3)n2)CC1] | 1-(4-(4-(6-amino-4-(trifluoromethyl)pyridin-3- yl)-6-morpholino-1,3,5-triazin-2-yl)piperazin-1- yl)-9-methyldec-8-ene-1,7-dione |
| 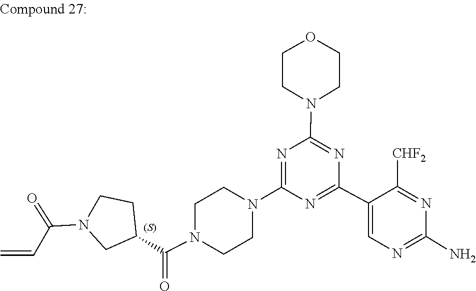 | (S)-1-(3-(4-(4-(2-amino-4- (difluoromethyl)pyrimidin-5-yl)-6-morpholino- 1,3,5-triazin-2-yl)piperazine-1- carbonyl)pyrrolidin-1-yl)prop-2-en-1-one |
| 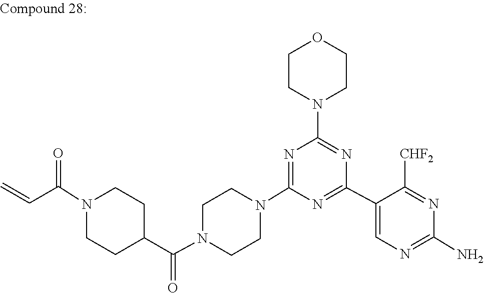 [SMILES: C=CC(=O)N1CCC(C(=O)N2CCN(c3nc(-c4cnc(N)nc4C(F)F)nc(N4CCOCC4)n3)CC2)CC1] | 1-(4-(4-(4-(2-amino-4- (difluoromethyl)pyrimidin-5-yl)-6-morpholino- 1,3,5-triazin-2-yl)piperazine-1- carbonyl)piperidin-1-yl)prop-2-en-1-one |
| 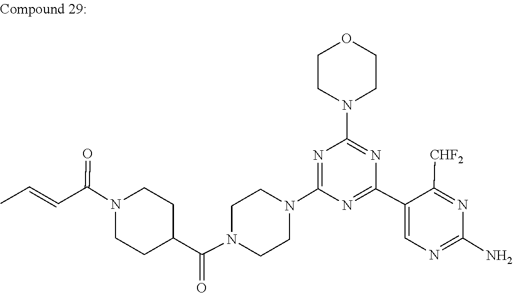 | (E)-1-(4-(4-(4-(2-amino-4- (difluoromethyl)pyrimidin-5-yl)-6-morpholino- 1,3,5-triazin-2-yl)piperazine-1- carbonyl)piperidin-1-yl)but-2-en-1-one |
| 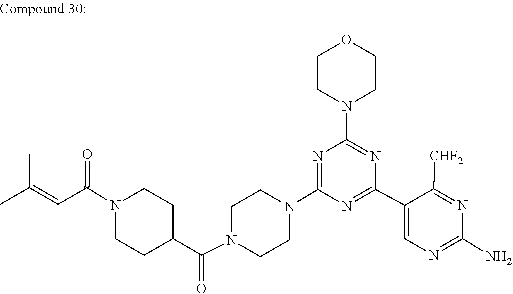 | 1-(4-(4-(4-(2-amino-4- (difluoromethyl)pyrimidin-5-yl)-6-morpholino- 1,3,5-triazin-2-yl)piperazine-1- carbonyl)piperidin-1-yl)-3-methylbut-2-en-1- one |
| 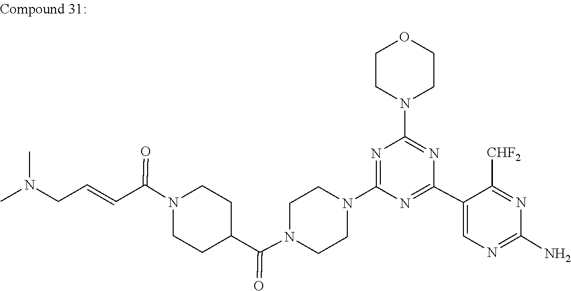 [SMILES: CN(C)C/C=C/C(=O)N1CCC(C(=O)N2CCN(c3nc(-c4cnc(N)nc4C(F)F)nc(N4CCOCC4)n3)CC2)CC1] | (E)-1-(4-(4-(4-(2-amino-4- (difluoromethyl)pyrimidin-5-yl)-6-morpholino- 1,3,5-triazin-2-yl)piperazine-1- carbonyl)piperidin-1-yl)-4-(dimethylamino)but- 2-en-1-one |
| 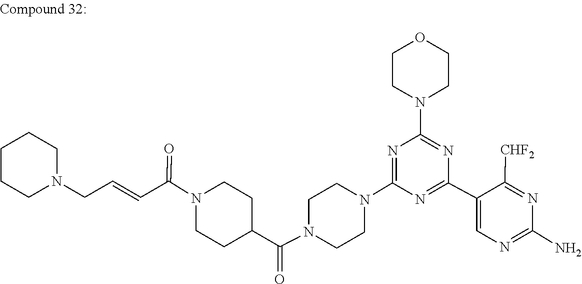 | (E)-1-(4-(4-(4-(2-amino-4- (difluoromethyl)pyrimidin-5-yl)-6-morpholino- 1,3,5-triazin-2-yl)piperazine-1- carbonyl)piperidin-1-yl)-4-(piperidin-1-yl)but-2- en-1-one |
| 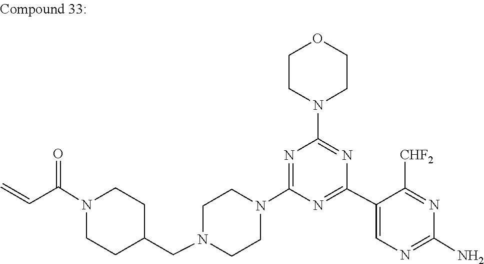 | 1-(4-((4-(4-(2-amino-4- (difluoromethyl)pyrimidin-5-yl)-6-morpholino- 1,3,5-triazin-2-yl)piperazin-1- yl)methyl)piperidin-1-yl)prop-2-en-1-one |
| 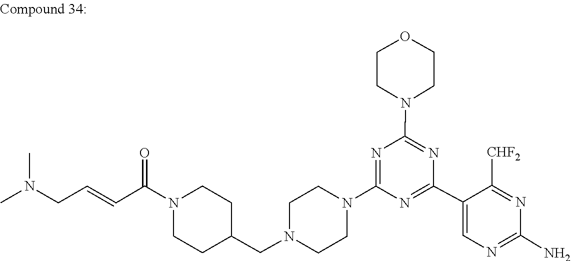 | (E)-1-(4-((4-(4-(2-amino-4- (difluoromethyl)pyrimidin-5-yl)-6-morpholino- 1,3,5-triazin-2-yl)piperazin-1- yl)methyl)piperidin-1-yl)-4-(dimethylamino)but- 2-en-1-one |
| 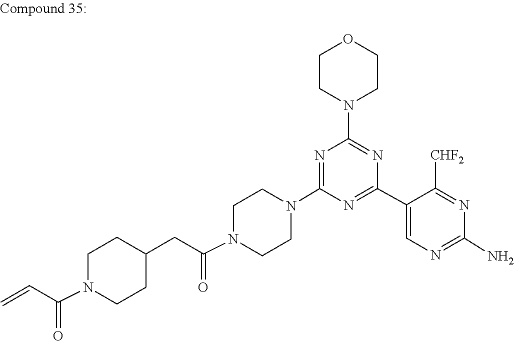 [SMILES: C=CC(=O)N1CCC(CC(=O)N2CCN(c3nc(-c4cnc(N)nc4C(F)F)nc(N4CCOCC4)n3)CC2)CC1] | 1-(4-(2-(4-(4-(2-amino-4- (difluoromethyl)pyrimidin-5-yl)-6-morpholino- 1,3,5-triazin-2-yl)piperazin-1-yl)-2- oxoethyl)piperidin-1-yl)prop-2-en-1-one |
| 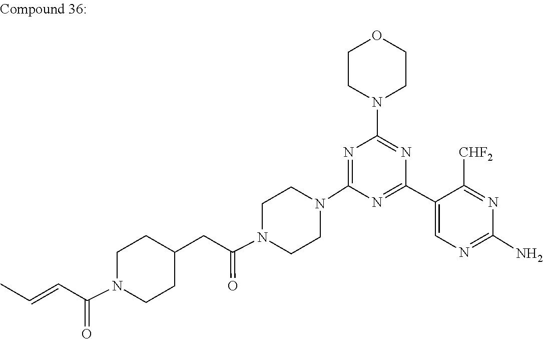 | (E)-1-(4-(2-(4-(4-(2-amino-4- (difluoromethyl)pyrimidin-5-yl)-6-morpholino- 1,3,5-triazin-2-yl)piperazin-1-yl)-2- oxoethyl)piperidin-1-yl)but-2-en-1-one |
| 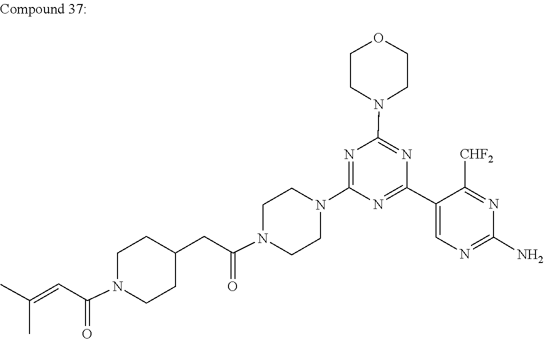 | 1-(4-(2-(4-(4-(2-amino-4- (difluoromethyl)pyrimidin-5-yl)-6-morpholino- 1,3,5-triazin-2-yl)piperazin-1-yl)-2- oxoethyl)piperidin-1-yl)-3-methylbut-2-en-1- one |
| 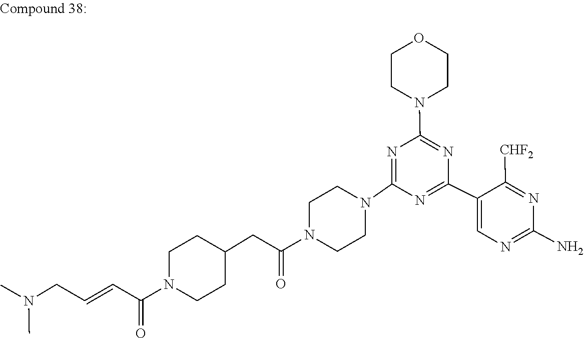 [SMILES: CN(C)C/C=C/C(=O)N1CCC(CC(=O)N2CCN(c3nc(-c4cnc(N)nc4C(F)F)nc(N4CCOCC4)n3)CC2)CC1] | (E)-1-(4-(2-(4-(4-(2-amino-4- (difluoromethyl)pyrimidin-5-yl)-6-morpholino- 1,3,5-triazin-2-yl)piperazin-1-yl)-2- oxoethyl)piperidin-1-yl)-4-(dimethylamino)but- 2-en-1-one |
| 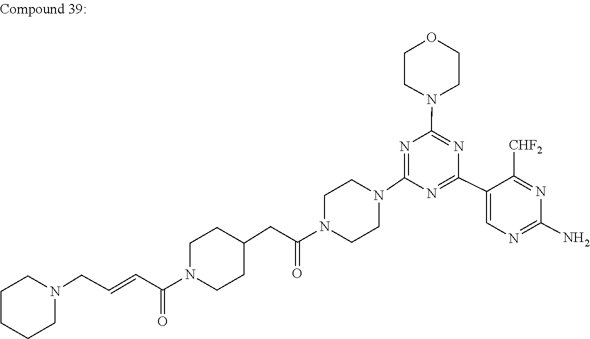 | (E)-1-(4-(2-(4-(4-(2-amino-4- (difluoromethyl)pyrimidin-5-yl)-6-morpholino- 1,3,5-triazin-2-yl)piperazin-1-yl)-2- oxoethyl)piperidin-1-yl)-4-(piperidin-1-yl)but-2- en-1-one |
| 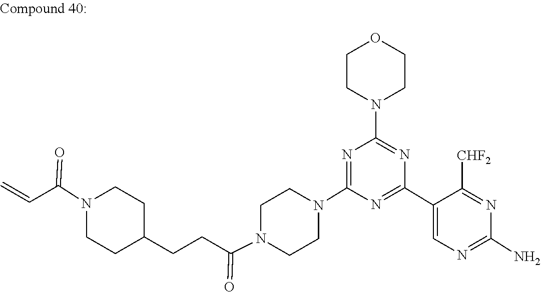 [SMILES: C=CC(=O)N1CCC(CCC(=O)N2CCN(c3nc(-c4cnc(N)nc4C(F)F)nc(N4CCOCC4)n3)CC2)CC1] | 1-(4-(3-(4-(4-(2-amino-4- (difluoromethyl)pyrimidin-5-yl)-6-morpholino- 1,3,5-triazin-2-yl)piperazin-1-yl)-3- oxopropyl)piperidin-1-yl)prop-2-en-1-one |
| 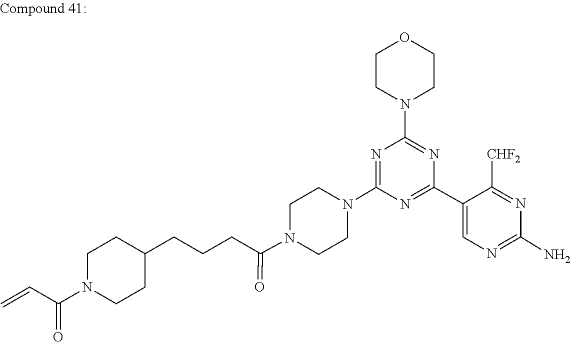 | 4-(1-acryloylpiperidin-4-yl)-1-(4-(4-(2-amino-4- (difluoromethyl)pyrimidin-5-yl)-6-morpholino- 1,3,5-triazin-2-yl)piperazin-1-yl)butan-1-one |
| 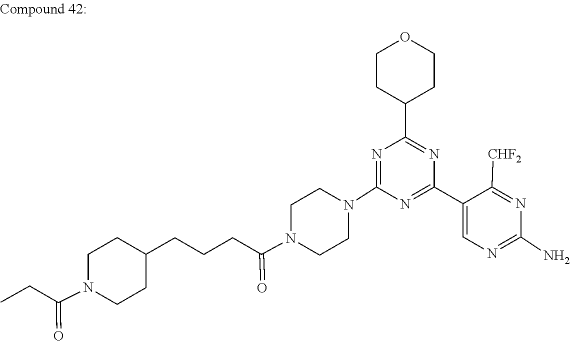 [SMILES: CCC(=O)N1CCC(CCCC(=O)N2CCN(c3nc(-c4cnc(N)nc4C(F)F)nc(C4CCOCC4)n3)CC2)CC1] | 1-(4-(4-(2-amino-4-(difluoromethyl)pyrimidin-5- yl)-6-morpholino-1,3,5-triazin-2-yl)piperazin-1- yl)-4-(1-propionylpiperidin-4-yl)butan-1-one |
| 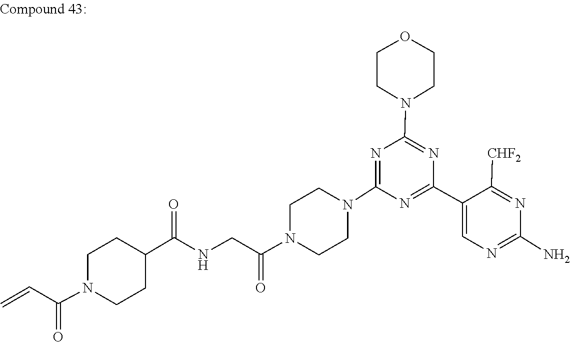 | 1-acryloyl-N-(2-(4-(4-(2-amino-4- (difluoromethyl)pyrimidin-5-yl)-6-morpholino- 1,3,5-triazin-2-yl)piperazin-1-yl)-2- oxoethyl)piperidine-4-carboxamide |
| 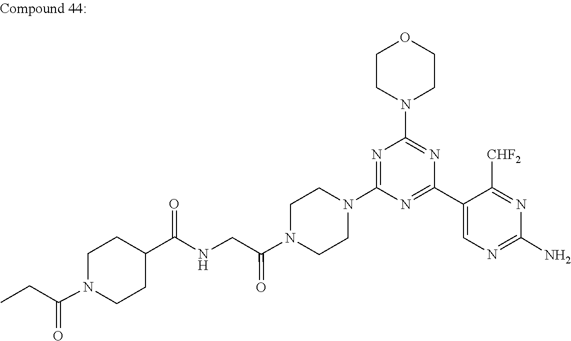 | N-(2-(4-(4-(2-amino-4- (difluoromethyl)pyrimidin-5-yl)-6-morpholino- 1,3,5-triazin-2-yl)piperazin-1-yl)-2-oxoethyl)-1- propionylpiperidine-4-carboxamide |
| 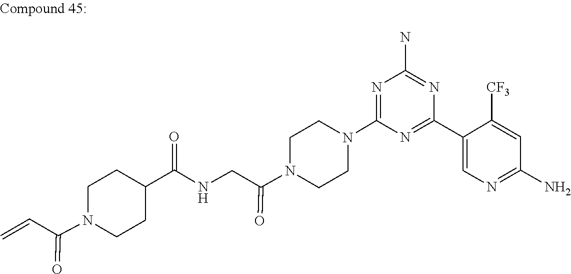 | 11-acryloyl-N-(2-(4-(4-(6-amino-4- (trifluoromethyl)pyridin-3-yl)-6-morpholino- 1,3,5-triazin-2-yl)piperazin-1-yl)-2- oxoethyl)piperidine-4-carboxamide |
| 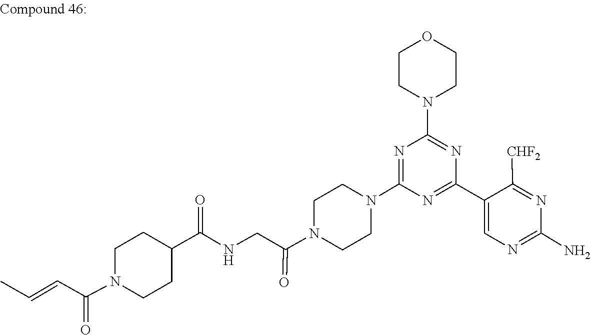 [SMILES: C/C=C/C(=O)N1CCC(C(=O)NCC(=O)N2CCN(c3nc(-c4cnc(N)nc4C(F)F)nc(N4CCOCC4)n3)CC2)CC1] | (E)-N-(2-(4-(4-(2-amino-4- (difluoromethyl)pyrimidin-5-yl)-6-morpholino- 1,3,5-triazin-2-yl)piperazin-1-yl)-2-oxoethyl)-1- (but-2-enoyl)piperidine-4-carboxamide |
| 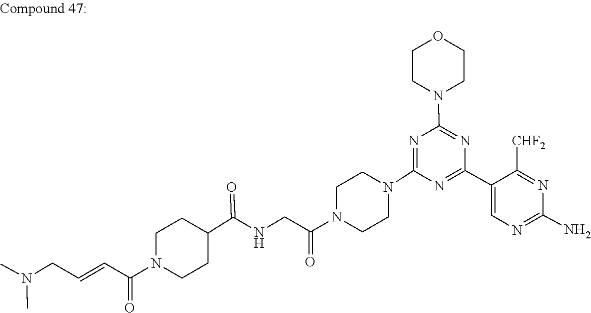 [SMILES: CN(C)C/C=C/C(=O)N1CCC(C(=O)NCC(=O)N2CCN(c3nc(-c4cnc(N)nc4C(F)F)nc(N4CCOCC4)n3)CC2)CC1] | (E)-N-(2-(4-(4-(2-amino-4- (difluoromethyl)pyrimidin-5-yl)-6-morpholino- 1,3,5-triazin-2-yl)piperazin-1-yl)-2-oxoethyl)-1- (4-(dimethylamino)but-2-enoyl)piperidine-4- carboxamide |
| 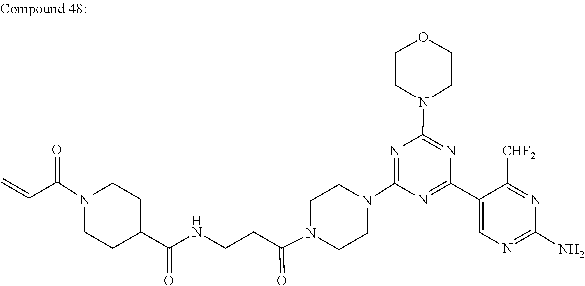 | 1-acryloyl-N-(3-(4-(4-(2-amino-4- (difluoromethyl)pyrimidin-5-yl)-6-morpholino- 1,3,5-triazin-2-yl)piperazin-1-yl)-3- oxopropyl)piperidine-4-carboxamide |
| 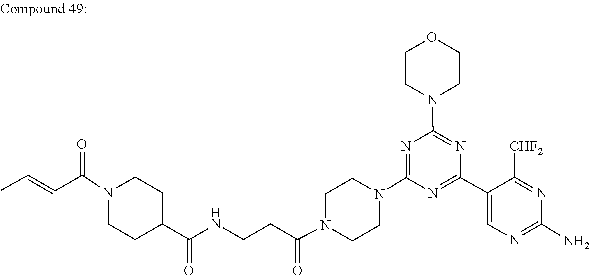 [SMILES: C/C=C/C(=O)N1CCC(C(=O)NCCC(=O)N2CCN(c3nc(-c4cnc(N)nc4C(F)F)nc(N4CCOCC4)n3)CC2)CC1] | (E)-N-(3-(4-(4-(2-amino-4- (difluoromethyl)pyrimidin-5-yl)-6-morpholino- 1,3,5-triazin-2-yl)piperazin-1-yl)-3-oxopropyl)- 1-(but-2-enoyl)piperidine-4-carboxamide |
| 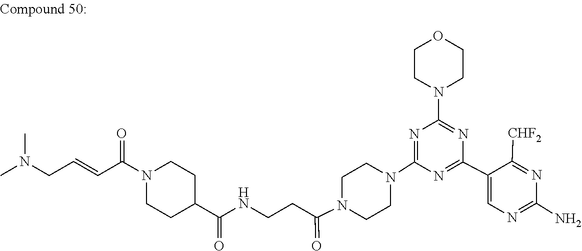 | (E)-N-(3-(4-(4-(2-amino-4- (difluoromethyl)pyrimidin-5-yl)-6-morpholino- 1,3,5-triazin-2-yl)piperazin-1-yl)-3-oxopropyl)- 1-(4-(dimethylamino)but-2-enoyl)piperidine-4- carboxamide |
| 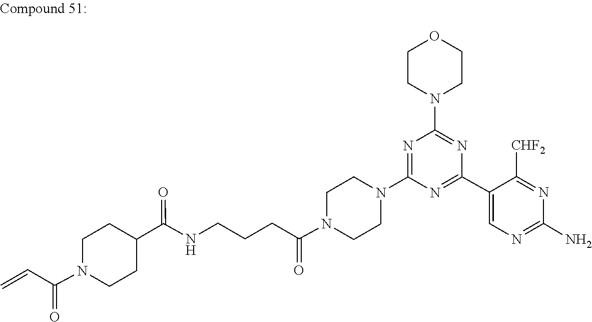 [SMILES: C=CC(=O)N1CCC(C(=O)NCCCC(=O)N2CCN(c3nc(-c4cnc(N)nc4C(F)F)nc(N4CCOCC4)n3)CC2)CC1] | 1-acryloyl-N-(4-(4-(4-(2-amino-4- (difluoromethyl)pyrimidin-5-yl)-6-morpholino- 1,3,5-triazin-2-yl)piperazin-1-yl)-4- oxobutyl)piperidine-4-carboxamide |
| 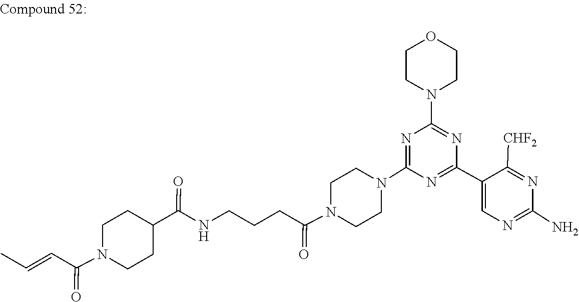 | (E)-N-(4-(4-(4-(2-amino-4- (difluoromethyl)pyrimidin-5-yl)-6-morpholino- 1,3,5-triazin-2-yl)piperazin-1-yl)-4-oxobutyl)-1- (but-2-enoyl)piperidine-4-carboxamide |
| 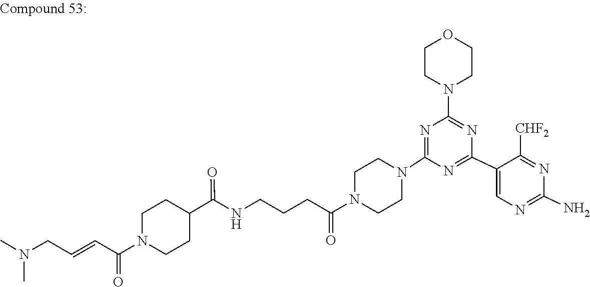 [SMILES: CN(C)C/C=C/C(=O)N1CCC(C(=O)NCCCC(=O)N2CCN(c3nc(-c4cnc(N)nc4C(F)F)nc(N4CCOCC4)n3)CC2)CC1] | (E)-N-(4-(4-(4-(2-amino-4- (difluoromethyl)pyrimidin-5-yl)-6-morpholino- 1,3,5-triazin-2-yl)piperazin-1-yl)-4-oxobutyl)-1- (4-(dimethylamino)but-2-enoyl)piperidine-4- carboxamide |
| 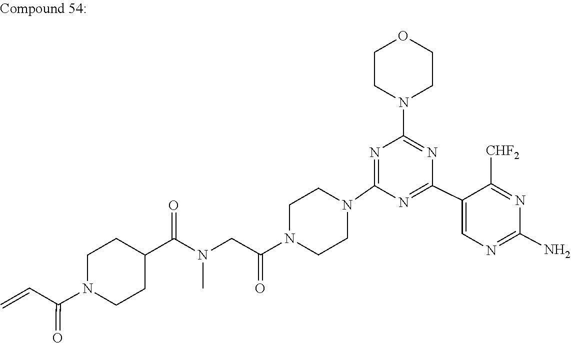 [SMILES: C=CC(=O)N1CCC(C(=O)N(C)CC(=O)N2CCN(c3nc(-c4cnc(N)nc4C(F)F)nc(N4CCOCC4)n3)CC2)CC1] | 1-acryloyl-N-(2-(4-(4-(2-amino-4- (difluoromethyl)pyrimidin-5-yl)-6-morpholino- 1,3,5-triazin-2-yl)piperazin-1-yl)-2-oxoethyl)-N- methylpiperidine-4-carboxamide |
| 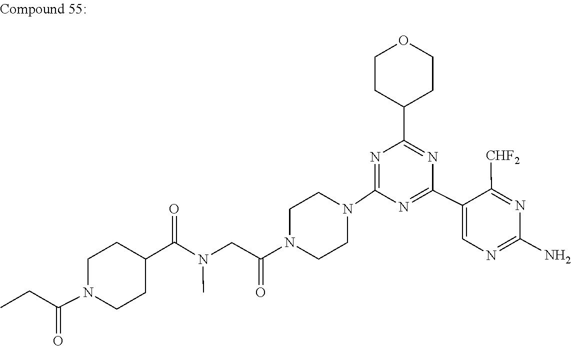 [SMILES: CCC(=O)N1CCC(C(=O)N(C)CC(=O)N2CCN(c3nc(-c4cnc(N)nc4C(F)F)nc(C4CCOCC4)n3)CC2)CC1] | N-(2-(4-(4-(2-amino-4- (difluoromethyl)pyrimidin-5-yl)-6-morpholino- 1,3,5-triazin-2-yl)piperazin-1-yl)-2-oxoethyl)-N- methyl-1-propionylpiperidine-4-carboxamide |
| 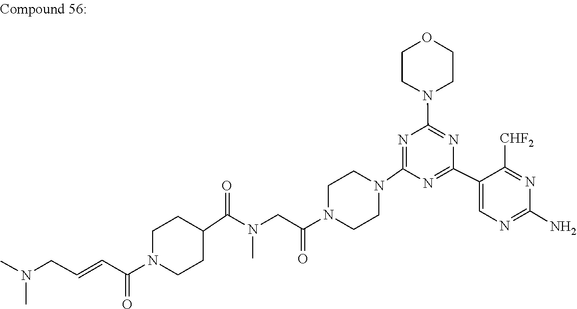 | (E)-N-(2-(4-(4-(2-amino-4- (difluoromethyl)pyrimidin-5-yl)-6-morpholino- 1,3,5-triazin-2-yl)piperazin-1-yl)-2-oxoethyl)-1- (4-(dimethylamino)but-2-enoyl)-N- methylpiperidine-4-carboxamide |
| 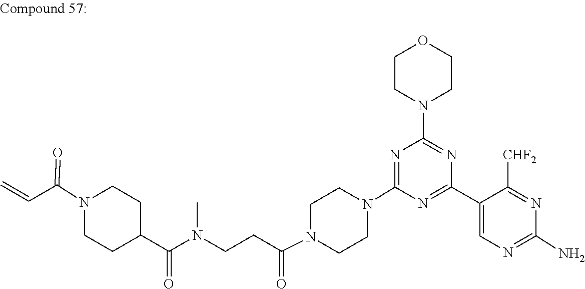 [SMILES: C=CC(=O)N1CCC(C(=O)N(C)CCC(=O)N2CCN(c3nc(-c4cnc(N)nc4C(F)F)nc(N4CCOCC4)n3)CC2)CC1] | 1-acryloyl-N-(3-(4-(4-(2-amino-4- (difluoromethyl)pyrimidin-5-yl)-6-morpholino- 1,3,5-triazin-2-yl)piperazin-1-yl)-3-oxopropyl)- N-methylpiperidine-4-carboxamide |
| 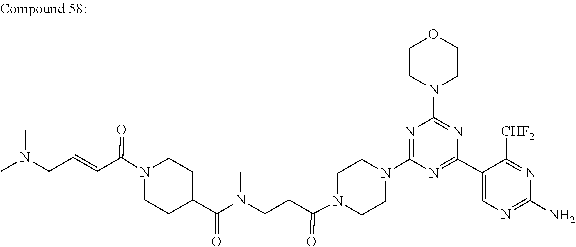 | (E)-N-(3-(4-(4-(2-amino-4- (difluoromethyl)pyrimidin-5-yl)-6-morpholino- 1,3,5-triazin-2-yl)piperazin-1-yl)-3-oxopropyl)- 1-(4-(dimethylamino)but-2-enoyl)-N- methylpiperidine-4-carboxamide |
| 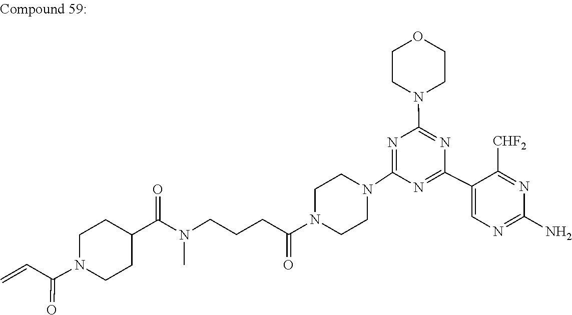 [SMILES: C=CC(=O)N1CCC(C(=O)N(C)CCCC(=O)N2CCN(c3nc(-c4cnc(N)nc4C(F)F)nc(N4CCOCC4)n3)CC2)CC1] | 1-acryloyl-N-(4-(4-(4-(2-amino-4- (difluoromethyl)pyrimidin-5-yl)-6-morpholino- 1,3,5-triazin-2-yl)piperazin-1-yl)-4-oxobutyl)-N- methylpiperidine-4-carboxamide |
| 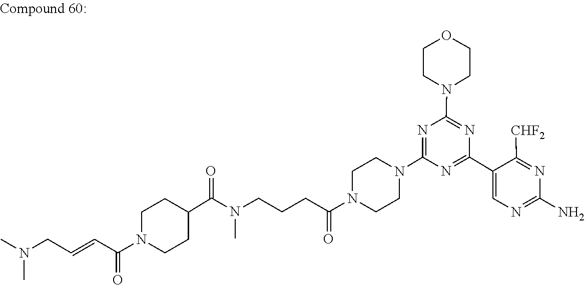 | (E)-N-(4-(4-(4-(2-amino-4- (difluoromethyl)pyrimidin-5-yl)-6-morpholino- 1,3,5-triazin-2-yl)piperazin-1-yl)-4-oxobutyl)-1- (4-(dimethylamino)but-2-enoyl)-N- methylpiperidine-4-carboxamide |
| 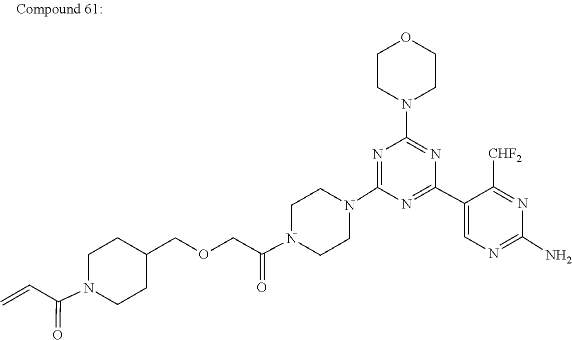 [SMILES: C=CC(=O)N1CCC(COCC(=O)N2CCN(c3nc(-c4cnc(N)nc4C(F)F)nc(N4CCOCC4)n3)CC2)CC1] | 1-(4-((2-(4-(4-(2-amino-4- (difluoromethyl)pyrimidin-5-yl)-6-morpholino- 1,3,5-triazin-2-yl)piperazin-1-yl)-2- oxoethoxy)methyl)piperidin-1-yl)prop-2-en-1- one |
| 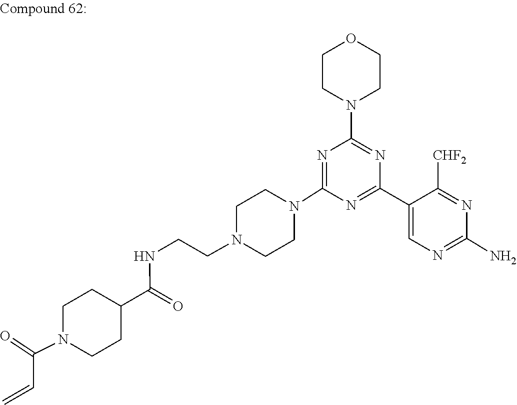 [SMILES: C=CC(=O)N1CCC(C(=O)NCCN2CCN(c3nc(-c4cnc(N)nc4C(F)F)nc(N4CCOCC4)n3)CC2)CC1] | 1-acryloyl-N-(2-(4-(4-(2-amino-4- (difluoromethyl)pyrimidin-5-yl)-6-morpholino- 1,3,5-triazin-2-yl)piperazin-1- yl)ethyl)piperidine-4-carboxamide |
| 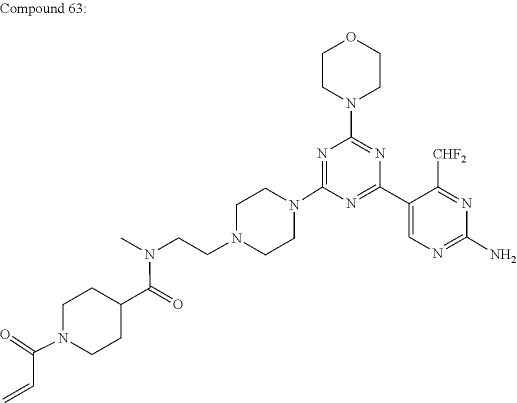 [SMILES: C=CC(=O)N1CCC(C(=O)N(C)CCN2CCN(c3nc(-c4cnc(N)nc4C(F)F)nc(N4CCOCC4)n3)CC2)CC1] | 1-(4-(((2-(4-(4-(2-amino-4- (difluoromethyl)pyrimidin-5-yl)-6-morpholino- 1,3,5-triazin-2-yl)piperazin-1-yl)-2- oxoethyl)(methyl)amino)methyl)piperidin-1- yl)prop-2-en-1-one |
| 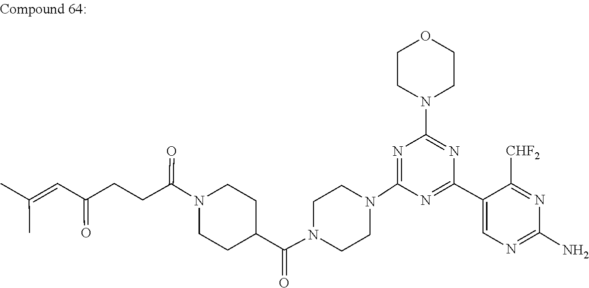 | 1-(4-(4-(4-(2-amino-4- (difluoromethyl)pyrimidin-5-yl)-6-morpholino- 1,3,5-triazin-2-yl)piperazine-1- carbonyl)piperidin-1-yl)-6-methylhept-5-ene- 1,4-dione |
| 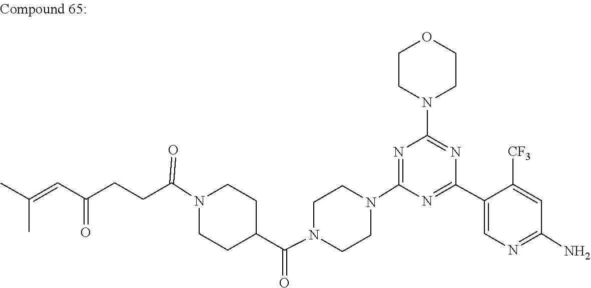 [SMILES: CC(C)=CC(=O)CCC(=O)N1CCC(C(=O)N2CCN(c3nc(-c4cnc(N)cc4C(F)(F)F)nc(N4CCOCC4)n3)CC2)CC1] | 1-(4-(4-(4-(6-amino-4-(trifluoromethyl)pyridin-3- yl)-6-morpholino-1,3,5-triazin-2-yl)piperazine- 1-carbonyl)piperidin-1-yl)-6-methylhept-5-ene- 1,4-dione |
| 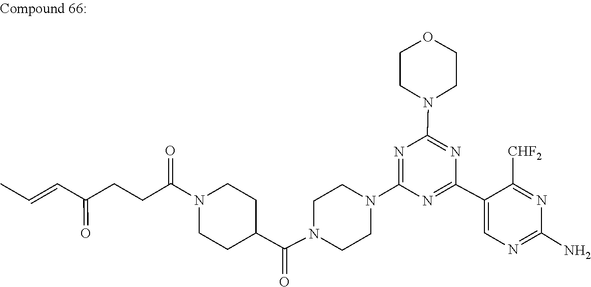 | (E)-1-(4-(4-(4-(2-amino-4- (difluoromethyl)pyrimidin-5-yl)-6-morpholino- 1,3,5-triazin-2-yl)piperazine-1- carbonyl)piperidin-1-yl)hept-5-ene-1,4-dione |
| 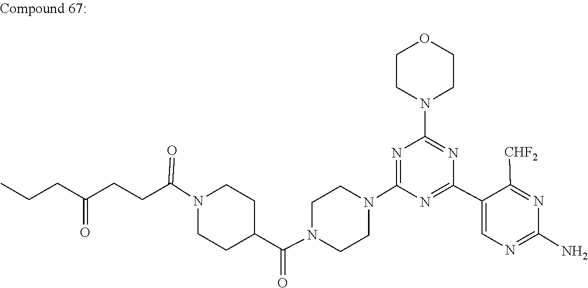 | 1-(4-(4-(4-(2-amino-4- (difluoromethyl)pyrimidin-5-yl)-6-morpholino- 1,3,5-triazin-2-yl)piperazine-1- carbonyl)piperidin-1-yl)heptane-1,4-dione |
| 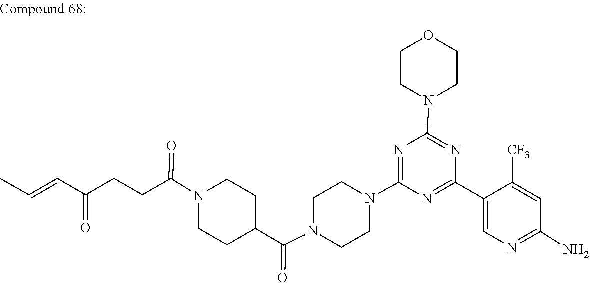 [SMILES: C/C=C/C(=O)CCC(=O)N1CCC(C(=O)N2CCN(c3nc(-c4cnc(N)cc4C(F)(F)F)nc(N4CCOCC4)n3)CC2)CC1] | (E)-1-(4-(4-(4-(6-amino-4- (trifluoromethyl)pyridin-3-yl)-6-morpholino- 1,3,5-triazin-2-yl)piperazine-1- carbonyl)piperidin-1-yl)hept-5-ene-1,4-dione |
| 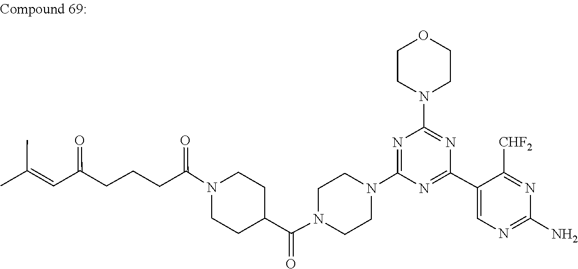 | 1-(4-(4-(4-(2-amino-4- (difluoromethyl)pyrimidin-5-yl)-6-morpholino- 1,3,5-triazin-2-yl)piperazine-1- carbonyl)piperidin-1-yl)-7-methyloct-6-ene- 1,5-dione |
| 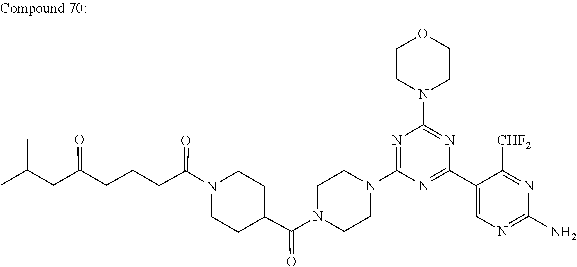 [SMILES: CC(C)CC(=O)CCCC(=O)N1CCC(C(=O)N2CCN(c3nc(-c4cnc(N)nc4C(F)F)nc(N4CCOCC4)n3)CC2)CC1] | 1-(4-(4-(4-(2-amino-4- (difluoromethyl)pyrimidin-5-yl)-6-morpholino- 1,3,5-triazin-2-yl)piperazine-1- carbonyl)piperidin-1-yl)-7-methyloctane-1,5- dione |
| 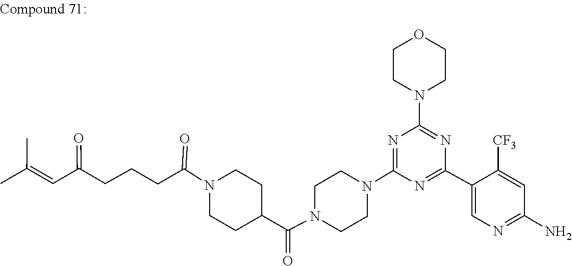 | 1-(4-(4-(4-(6-amino-4-(trifluoromethyl)pyridin- 3-yl)-6-morpholino-1,3,5-triazin-2- yl)piperazine-1-carbonyl)piperidin-1-yl)-7- methyloct-6-ene-1,5-dione |
| 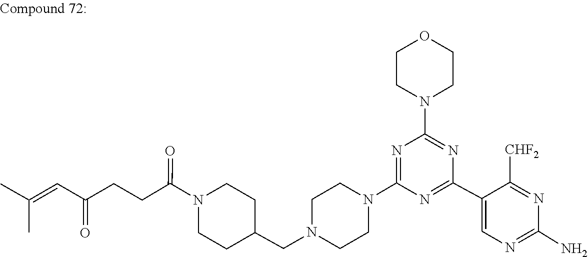 | 1-(4-((4-(4-(2-amino-4- (difluoromethyl)pyrimidin-5-yl)-6-morpholino- 1,3,5-triazin-2-yl)piperazin-1- yl)methyl)piperidin-1-yl)-6-methylhept-5-ene- 1,4-dione |
| 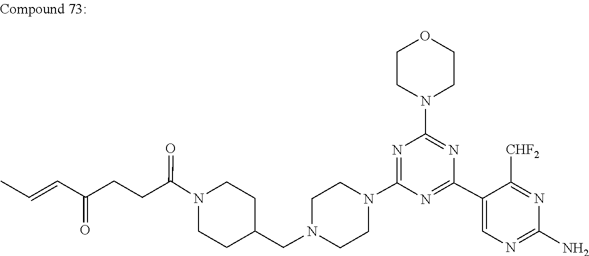 | (E)-1-(4-((4-(4-(2-amino-4- (difluoromethyl)pyrimidin-5-yl)-6-morpholino- 1,3,5-triazin-2-yl)piperazin-1- yl)methyl)piperidin-1-yl)hept-5-ene-1,4-dione |
| 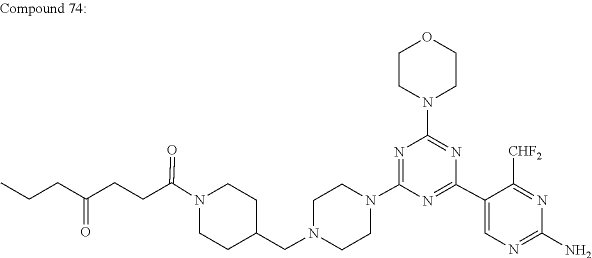 | 1-(4-((4-(4-(2-amino-4- (difluoromethyl)pyrimidin-5-yl)-6-morpholino- 1,3,5-triazin-2-yl)piperazin-1- yl)methyl)piperidin-1-yl)heptane-1,4-dione |
| 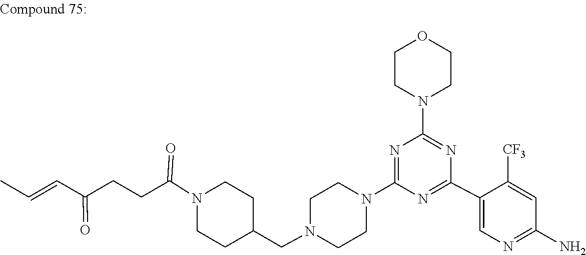 [SMILES: C/C=C/C(=O)CCC(=O)N1CCC(CN2CCN(c3nc(-c4cnc(N)cc4C(F)(F)F)nc(N4CCOCC4)n3)CC2)CC1] | (E)-1-(4-((4-(4-(6-amino-4- (trifluoromethyl)pyridin-3-yl)-6-morpholino- 1,3,5-triazin-2-yl)piperazin-1- yl)methyl)piperidin-1-yl)hept-5-ene-1,4-dione |
| 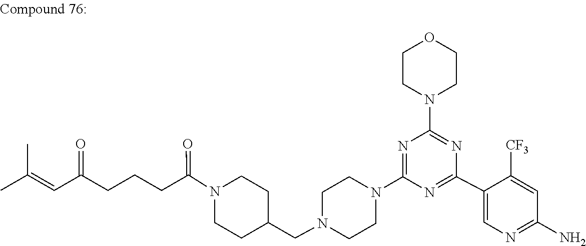 [SMILES: CC(C)=CC(=O)CCCC(=O)N1CCC(CN2CCN(c3nc(-c4cnc(N)cc4C(F)(F)F)nc(N4CCOCC4)n3)CC2)CC1] | 1-(4-((4-(4-(2-amino-4- (difluoromethyl)pyrimidin-5-yl)-6-morpholino- 1,3,5-triazin-2-yl)piperazin-1- yl)methyl)piperidin-1-yl)-7-methyloct-6-ene- 1,5-dione |
| 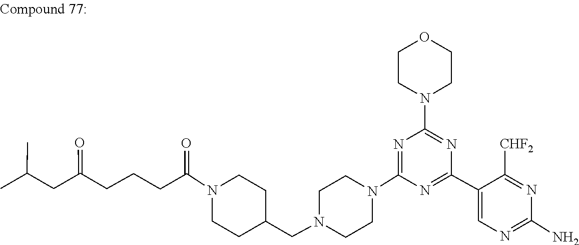 | 1-(4-((4-(4-(2-amino-4- (difluoromethyl)pyrimidin-5-yl)-6-morpholino- 1,3,5-triazin-2-yl)piperazin-1- yl)methyl)piperidin-1-yl)-7-methyloctane-1,5- dione |
| 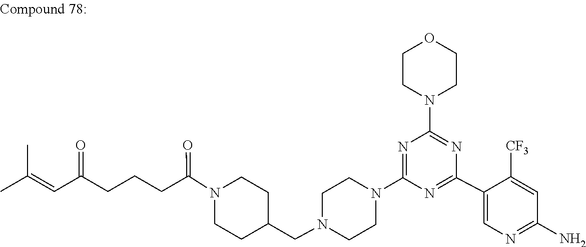 [SMILES: CC(C)=CC(=O)CCCC(=O)N1CCC(CN2CCN(c3nc(-c4cnc(N)cc4C(F)(F)F)nc(N4CCOCC4)n3)CC2)CC1] | 1-(4-((4-(4-(6-amino-4-(trifluoromethyl)pyridin- 3-yl)-6-morpholino-1,3,5-triazin-2-yl)piperazin- 1-yl)methyl)piperidin-1-yl)-7-methyloct-6-ene- 1,5-dione |
| 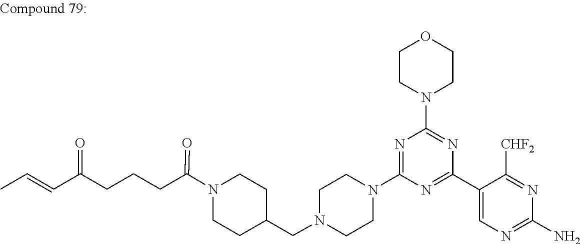 | (E)-1-(4-((4-(4-(2-amino-4- (difluoromethyl)pyrimidin-5-yl)-6-morpholino- 1,3,5-triazin-2-yl)piperazin-1- yl)methyl)piperidin-1-yl)oct-6-ene-1,5-dione |
| 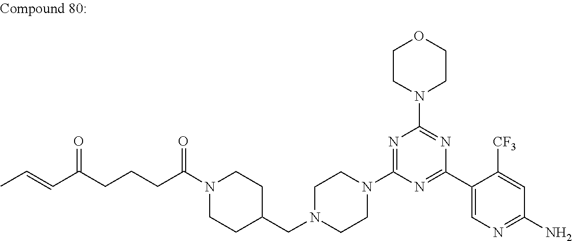 | (E)-1-(4-((4-(4-(6-amino-4- (trifluoromethyl)pyridin-3-yl)-6-morpholino- 1,3,5-triazin-2-yl)piperazin-1- yl)methyl)piperidin-1-yl)oct-6-ene-1,5-dione |
| 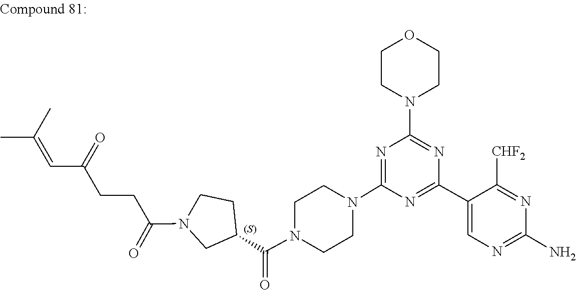 | (S)-1-(3-(4-(4-(2-amino-4- (difluoromethyl)pyrimidin-5-yl)-6-morpholino- 1,3,5-triazin-2-yl)piperazine-1- carbonyl)pyrrolidin-1-yl)-6-methylhept-5-ene- 1,4-dione |
| 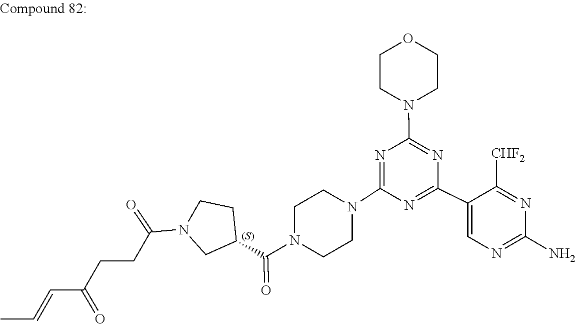 | (S,E)-1-(3-(4-(4-(2-amino-4- (difluoromethyl)pyrimidin-5-yl)-6-morpholino- 1,3,5-triazin-2-yl)piperazine-1- carbonyl)pyrrolidin-1-yl)hept-5-ene-1,4-dione |
| 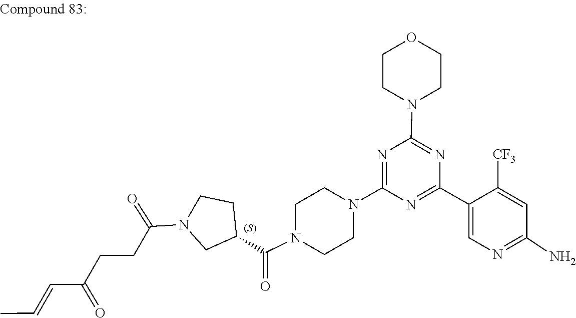 | (S,E)-1-(3-(4-(4-(6-amino-4- (trifluoromethyl)pyridin-3-yl)-6-morpholino- 1,3,5-triazin-2-yl)piperazine-1- carbonyl)pyrrolidin-1-yl)hept-5-ene-1,4-dione |
| 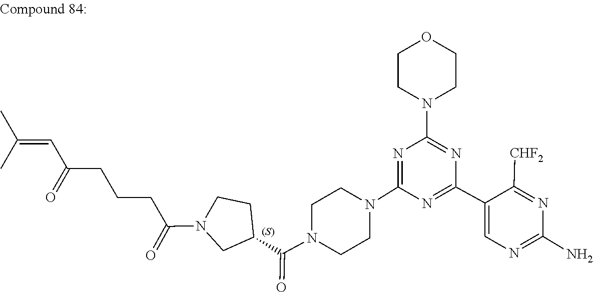 | (S)-1-(3-(4-(4-(2-amino-4- (difluoromethyl)pyrimidin-5-yl)-6-morpholino- 1,3,5-triazin-2-yl)piperazine-1- carbonyl)pyrrolidin-1-yl)-7-methyloct-6-ene- 1,5-dione |
| 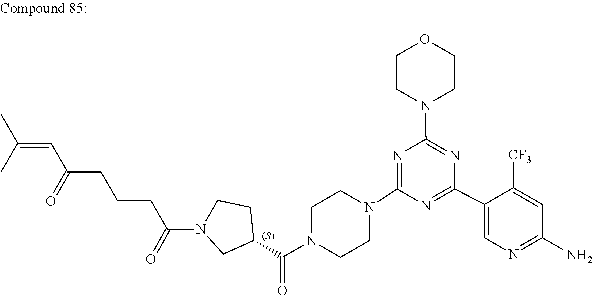 | (S)-1-(3-(4-(4-(6-amino-4- (trifluoromethyl)pyridin-3-yl)-6-morpholino- 1,3,5-triazin-2-yl)piperazine-1- carbonyl)pyrrolidin-1-yl)-7-methyloct-6-ene- 1,5-dione |
| 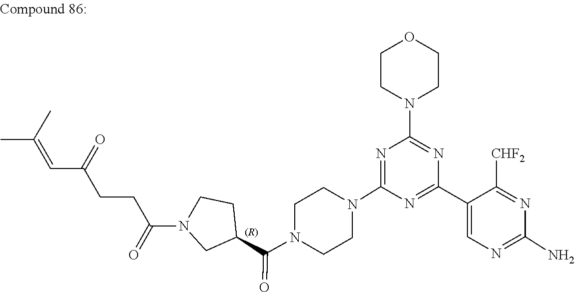 | (R)-1-(3-(4-(4-(2-amino-4- (difluoromethyl)pyrimidin-5-yl)-6-morpholino- 1,3,5-triazin-2-yl)piperazine-1- carbonyl)pyrrolidin-1-yl)-6-methylhept-5-ene- 1,4-dione |
| 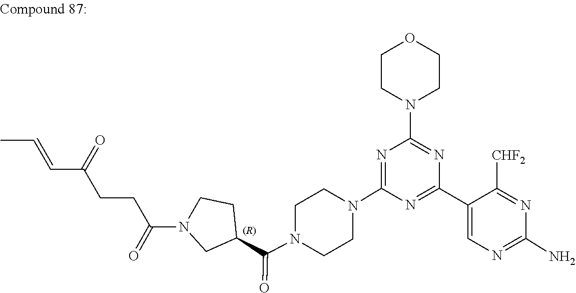 | (R,E)-1-(3-(4-(4-(2-amino-4- (difluoromethyl)pyrimidin-5-yl)-6-morpholino- 1,3,5-triazin-2-yl)piperazine-1- carbonyl)pyrrolidin-1-yl)hept-5-ene-1,4-dione |
| 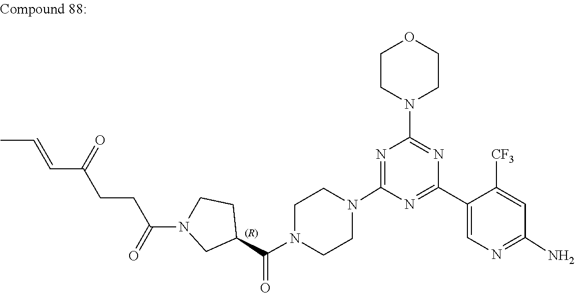 | (R,E)-1-(3-(4-(4-(6-amino-4- (trifluoromethyl)pyridin-3-yl)-6-morpholino- 1,3,5-triazin-2-yl)piperazine-1- carbonyl)pyrrolidin-1-yl)hept-5-ene-1,4-dione |
| 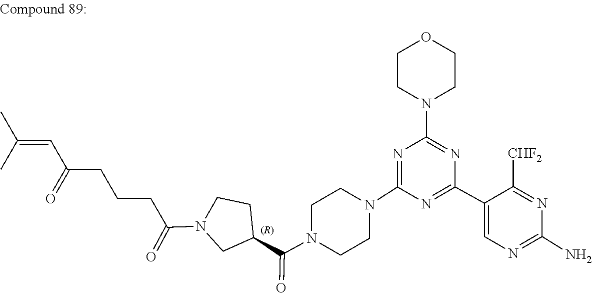 | (R)-1-(3-(4-(4-(2-amino-4- (difluoromethyl)pyrimidin-5-yl)-6-morpholino- 1,3,5-triazin-2-yl)piperazine-1- carbonyl)pyrrolidin-1-yl)-7-methyloct-6-ene- 1,5-dione |
| 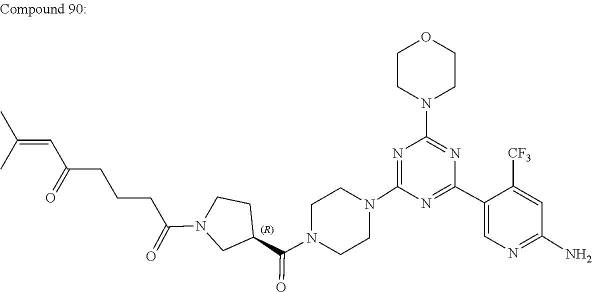 | (R)-1-(3-(4-(4-(6-amino-4- (trifluoromethyl)pyridin-3-yl)-6-morpholino- 1,3,5-triazin-2-yl)piperazine-1- carbonyl)pyrrolidin-1-yl)-7-methyloct-6-ene- 1,5-dione |
| 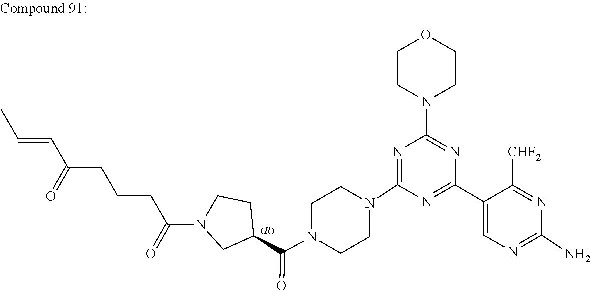 | (R,E)-1-(3-(4-(4-(2-amino-4- (difluoromethyl)pyrimidin-5-yl)-6-morpholino- 1,3,5-triazin-2-yl)piperazine-1- carbonyl)pyrrolidin-1-yl)oct-6-ene-1,5-dione |
| 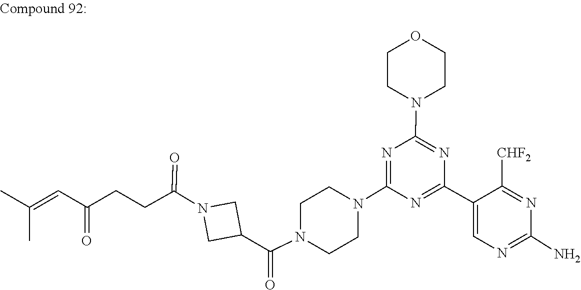 [SMILES: CC(C)=CC(=O)CCC(=O)N1CC(C(=O)N2CCN(c3nc(-c4cnc(N)nc4C(F)F)nc(N4CCOCC4)n3)CC2)C1] | 1-(3-(4-(4-(2-amino-4- (difluoromethyl)pyrimidin-5-yl)-6-morpholino- 1,3,5-triazin-2-yl)piperazine-1- carbonyl)azetidin-1-yl)-6-methylhept-5-ene- 1,4-dione |
| 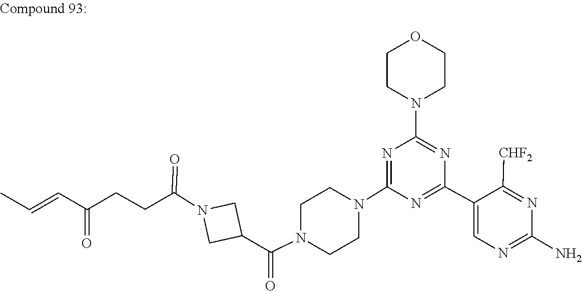 [SMILES: C/C=C/C(=O)CCC(=O)N1CC(C(=O)N2CCN(c3nc(-c4cnc(N)nc4C(F)F)nc(N4CCOCC4)n3)CC2)C1] | (E)-1-(3-(4-(4-(2-amino-4- (difluoromethyl)pyrimidin-5-yl)-6-morpholino- 1,3,5-triazin-2-yl)piperazine-1- carbonyl)azetidin-1-yl)hept-5-ene-1,4-dione |
| 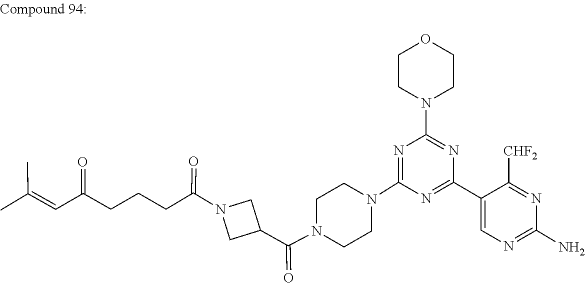 [SMILES: CC(C)=CC(=O)CCCC(=O)N1CC(C(=O)N2CCN(c3nc(-c4cnc(N)nc4C(F)F)nc(N4CCOCC4)n3)CC2)C1] | 1-(3-(4-(4-(2-amino-4- (difluoromethyl)pyrimidin-5-yl)-6-morpholino- 1,3,5-triazin-2-yl)piperazine-1- carbonyl)azetidin-1-yl)-7-methyloct-6-ene-1,5- dione |
| 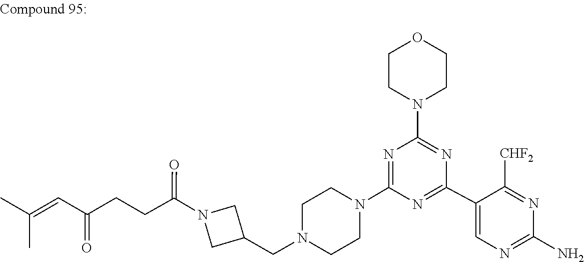 [SMILES: CC(C)=CC(=O)CCC(=O)N1CC(CN2CCN(c3nc(-c4cnc(N)nc4C(F)F)nc(N4CCOCC4)n3)CC2)C1] | 1-(3-((4-(4-(2-amino-4- (difluoromethyl)pyrimidin-5-yl)-6-morpholino- 1,3,5-triazin-2-yl)piperazin-1- yl)methyl)azetidin-1-yl)-6-methylhept-5-ene- 1,4-dione |
| 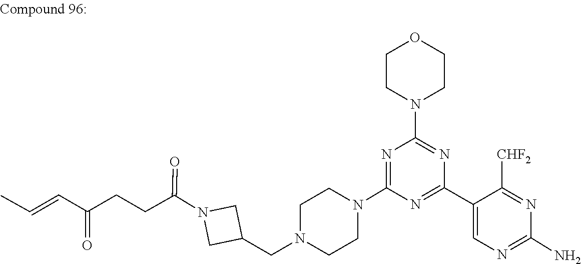 [SMILES: C/C=C/C(=O)CCC(=O)N1CC(CN2CCN(c3nc(-c4cnc(N)nc4C(F)F)nc(N4CCOCC4)n3)CC2)C1] | (E)-1-(3-((4-(4-(2-amino-4- (difluoromethyl)pyrimidin-5-yl)-6- morpholino-1,3,5-triazin-2-yl)piperazin- 1-yl)methyl)azetidin-1-yl)hept-5-ene- 1,4-dione |
| 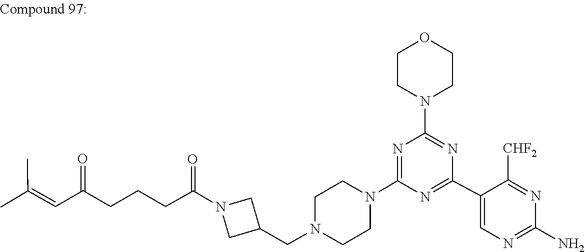 [SMILES: CC(C)=CC(=O)CCCC(=O)N1CC(CN2CCN(c3nc(-c4cnc(N)nc4C(F)F)nc(N4CCOCC4)n3)CC2)C1] | 1-(3-((4-(4-(2-amino-4- (difluoromethyl)pyrimidin-5-yl)-6- morpholino-1,3,5-triazin-2-yl)piperazin- 1-yl)methyl)azetidin-1-yl)-7-methyloct- 6-ene-1,5-dione |
| 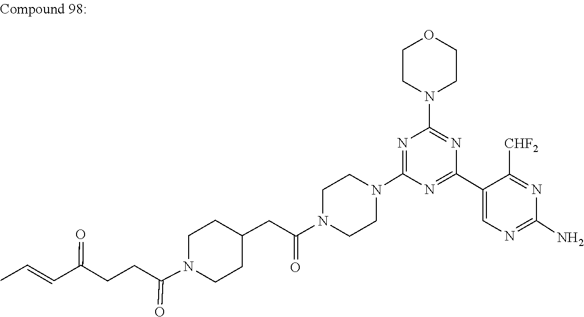 [SMILES: C/C=C/C(=O)CCC(=O)N1CCC(CC(=O)N2CCN(c3nc(-c4cnc(N)nc4C(F)F)nc(N4CCOCC4)n3)CC2)CC1] | (E)-1-(4-(2-(4-(4-(2-amino-4- (difluoromethyl)pyrimidin-5-yl)-6- morpholino-1,3,5-triazin-2-yl)piperazin- 1-yl)-2-oxoethyl)piperidin-1-yl)hept-5- ene-1,4-dione |
| 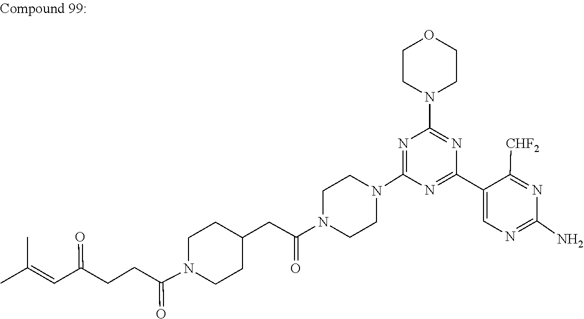 [SMILES: CC(C)=CC(=O)CCC(=O)N1CCC(CC(=O)N2CCN(c3nc(-c4cnc(N)nc4C(F)F)nc(N4CCOCC4)n3)CC2)CC1] | 1-(4-(2-(4-(4-(2-amino-4- (difluoromethyl)pyrimidin-5-yl)-6- morpholino-1,3,5-triazin-2-yl)piperazin- 1-yl)-2-oxoethyl)piperidin-1-yl)-6- methylhept-5-ene-1,4-dione |
| 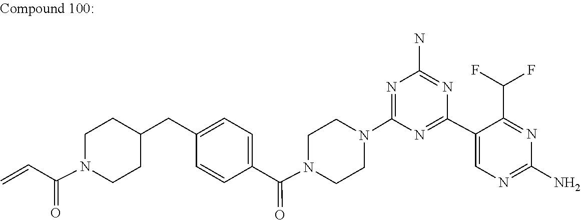 | 1-(4-(4-(4-(4-(2-amino-4- (difluoromethyl)pyrimidin-5-yl)-6- morpholino-1,3,5-triazin-2- yl)piperazine-1- carbonyl)benzyl)piperidin-1-yl)prop-2- en-1-one |
| 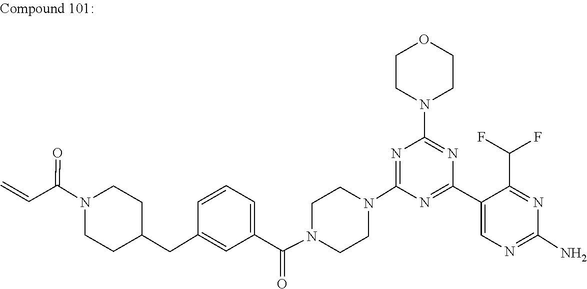 [SMILES: C=CC(=O)N1CCC(Cc2cccc(C(=O)N3CCN(c4nc(-c5cnc(N)nc5C(F)F)nc(N5CCOCC5)n4)CC3)c2)CC1] | 1-(4-(3-(4-(4-(2-amino-4- (difluoromethyl)pyrimidin-5-yl)-6- morpholino-1,3,5-triazin-2- yl)piperazine-1- carbonyl)benzyl)piperidin-1-yl)prop-2- en-1-one |
| 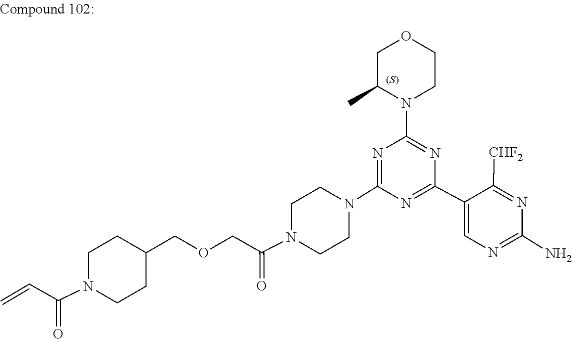 | (S)-1-(4-((2-(4-(4-(2-amino-4- (difluoromethyl)pyrimidin-5-yl)-6-(3- methylmorpholino)-1,3,5-triazin-2- yl)piperazin-1-yl)-2- oxoethoxy)methyl)piperidin-1-yl)prop- 2-en-1-one |
| 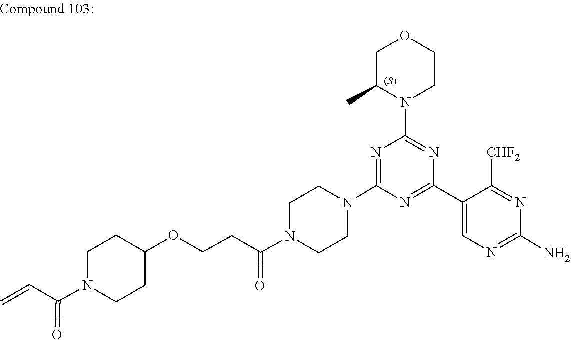 | (S)-1-(4-(3-(4-(4-(2-amino-4- (difluoromethyl)pyrimidin-5-yl)-6-(3- methylmorpholino)-1,3,5-triazin-2- yl)piperazin-1-yl)-3- oxopropoxy)piperidin-1-yl)prop-2-en-1- one |
| 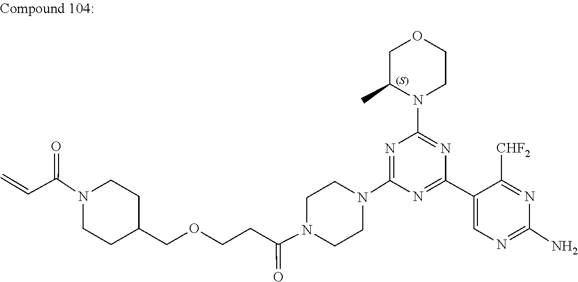 | (S)-1-(4-((3-(4-(4-(2-amino-4- (difluoromethyl)pyrimidin-5-yl)-6-(3- methylmorpholino)-1,3,5-triazin-2- yl)piperazin-1-yl)-3- oxopropoxy)methyl)piperidin-1-yl)prop- 2-en-1-one |
| 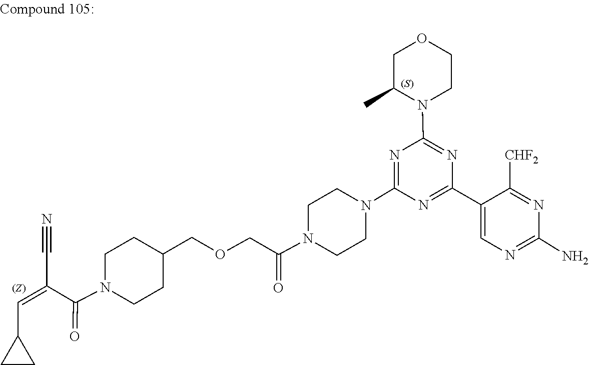 | (S,Z)-2-(4-((2-(4-(4-(2-amino-4- (difluoromethyl)pyrimidin-5-yl)-6-(3- methylmorpholino)-1,3,5-triazin-2- yl)piperazin-1-yl)-2- oxoethoxy)methyl)piperidine-1- carbonyl)-3-cyclopropylacrylonitrile |
| 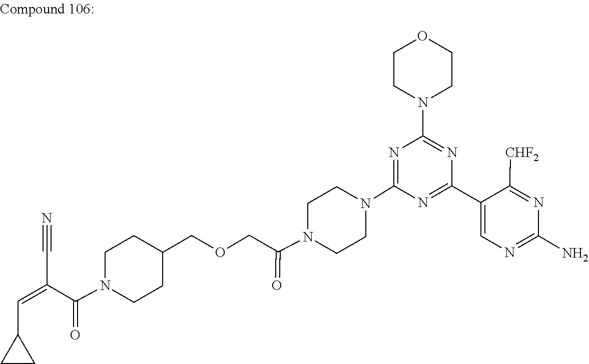 [SMILES: N#C/C(=C/C1CC1)C(=O)N1CCC(COCC(=O)N2CCN(c3nc(-c4cnc(N)nc4C(F)F)nc(N4CCOCC4)n3)CC2)CC1] | (Z)-2-(4-((2-(4-(4-(2-amino-4- (difluoromethyl)pyrimidin-5-yl)-6- morpholino-1,3,5-triazin-2-yl)piperazin- 1-yl)-2-oxoethoxy)methyl)piperidine-1- carbonyl)-3-cyclopropylacrylonitrile |
| 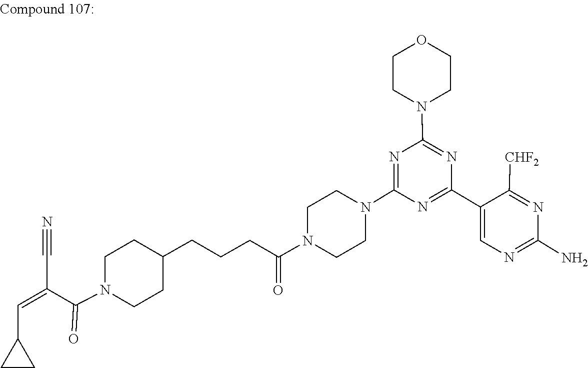 [SMILES: N#C/C(=C/C1CC1)C(=O)N1CCC(CCCC(=O)N2CCN(c3nc(-c4cnc(N)nc4C(F)F)nc(N4CCOCC4)n3)CC2)CC1] | (Z)-2-(4-(4-(4-(4-(2-amino-4- (difluoromethyl)pyrimidin-5-yl)-6- morpholino-1,3,5-triazin-2-yl)piperazin- 1-yl)-4-oxobutyl)piperidine-1-carbonyl)- 3-cyclopropylacrylonitrile |
| 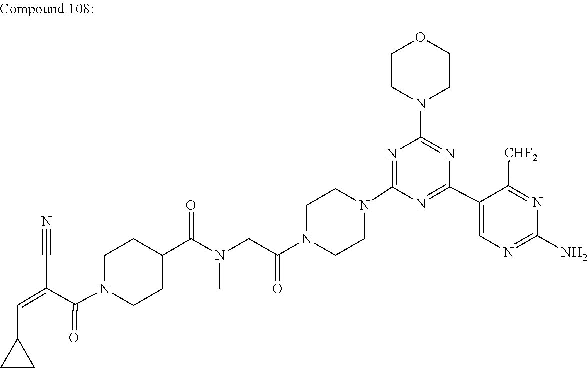 [SMILES: CN(CC(=O)N1CCN(c2nc(-c3cnc(N)nc3C(F)F)nc(N3CCOCC3)n2)CC1)C(=O)C1CCN(C(=O)/C(C#N)=C\C2CC2)CC1] | (Z)-N-(2-(4-(4-(2-amino-4- (difluoromethyl)pyrimidin-5-yl)-6- morpholino-1,3,5-triazin-2-yl)piperazin- 1-yl)-2-oxoethyl)-1-(2-cyano-3- cyclopropylacryloyl)-N- methylpiperidine-4-carboxamide |
| 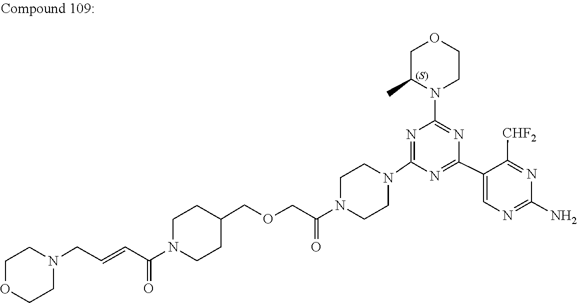 | (S,E)-1-(4-((2-(4-(4-(2-amino-4- (difluoromethyl)pyrimidin-5-yl)-6-(3- methylmorpholino)-1,3,5-triazin-2- yl)piperazin-1-yl)-2- oxoethoxy)methyl)piperidin-1-yl)-4- morpholinobut-2-en-1-one |
| 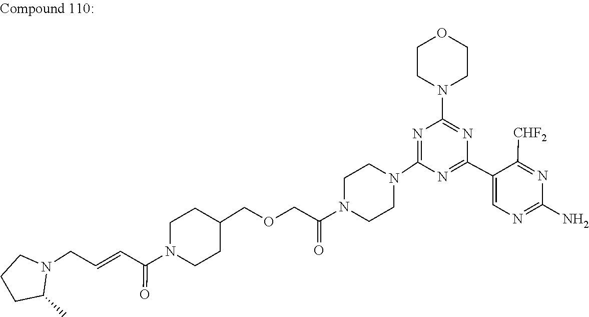 [SMILES: C[C@@H]1CCCN1C/C=C/C(=O)N1CCC(COCC(=O)N2CCN(c3nc(-c4cnc(N)nc4C(F)F)nc(N4CCOCC4)n3)CC2)CC1] | (R,E)-1-(4-((2-(4-(4-(2-amino-4- (difluoromethyl)pyrimidin-5-yl)-6- morpholino-1,3,5-triazin-2-yl)piperazin- 1-yl)-2-oxoethoxy)methyl)piperidin-1- yl)-4-(2-methylpyrrolidin-1-yl)but-2-en- 1-one |
| 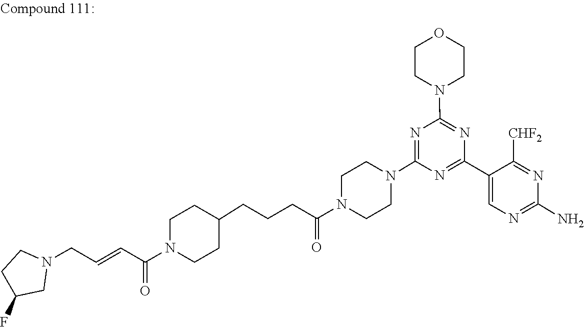 [SMILES: Nc1ncc(-c2nc(N3CCOCC3)nc(N3CCN(C(=O)CCCC4CCN(C(=O)/C=C/CN5CC[C@H](F)C5)CC4)CC3)n2)c(C(F)F)n1] | (S,E)-1-(4-(4-(4-(4-(2-amino-4- (difluoromethyl)pyrimidin-5-yl)-6- morpholino-1,3,5-triazin-2-yl)piperazin- 1-yl)-4-oxobutyl)piperidin-1-yl)-4-(3- fluoropyrrolidin-1-yl)but-2-en-1-one |
| 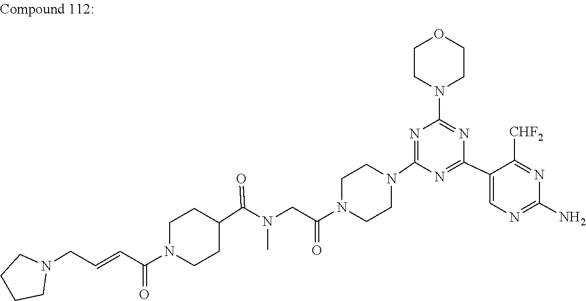 [SMILES: CN(CC(=O)N1CCN(c2nc(-c3cnc(N)nc3C(F)F)nc(N3CCOCC3)n2)CC1)C(=O)C1CCN(C(=O)/C=C/CN2CCCC2)CC1] | (E)-N-(2-(4-(4-(2-amino-4- (difluoromethyl)pyrimidin-5-yl)-6- morpholino-1,3,5-triazin-2-yl)piperazin- 1-yl)-2-oxoethyl)-N-methyl-1-(4- (pyrrolidin-1-yl)but-2-enoyl)piperidine- 4-carboxamide |
| 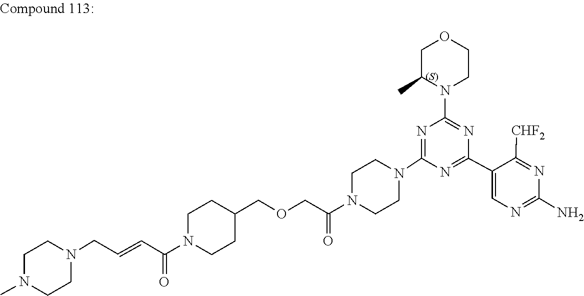 | (S,E)-1-(4-((2-(4-(4-(2-amino-4- (difluoromethyl)pyrimidin-5-yl)-6-(3- methylmorpholino)-1,3,5-triazin-2- yl)piperazin-1-yl)-2- oxoethoxy)methyl)piperidin-1-yl)-4-(4- methylpiperazin-1-yl)but-2-en-1-one |
| 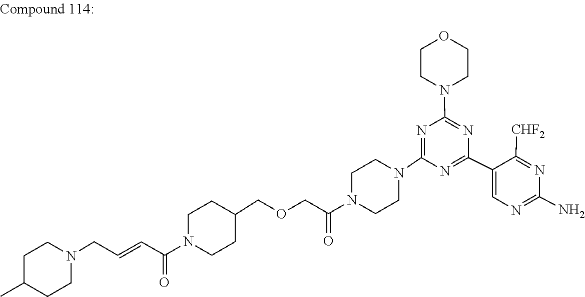 [SMILES: CC1CCN(C/C=C/C(=O)N2CCC(COCC(=O)N3CCN(c4nc(-c5cnc(N)nc5C(F)F)nc(N5CCOCC5)n4)CC3)CC2)CC1] | (E)-1-(4-((2-(4-(4-(2-amino-4- (difluoromethyl)pyrimidin-5-yl)-6- morpholino-1,3,5-triazin-2-yl)piperazin- 1-yl)-2-oxoethoxy)methyl)piperidin-1- yl)-4-(4-methylpiperidin-1-yl)but-2-en-1- one |
| 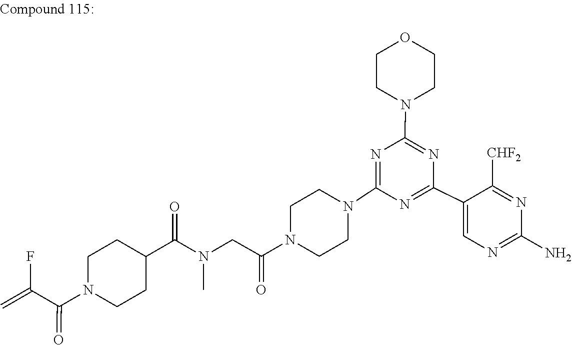 [SMILES: C=C(F)C(=O)N1CCC(C(=O)N(C)CC(=O)N2CCN(c3nc(-c4cnc(N)nc4C(F)F)nc(N4CCOCC4)n3)CC2)CC1] | N-(2-(4-(4-(2-amino-4- (difluoromethyl)pyrimidin-5-yl)-6- morpholino-1,3,5-triazin-2-yl)piperazin- 1-yl)-2-oxoethyl)-1-(2-fluoroacryloyl)-N- methylpiperidine-4-carboxamide |
| 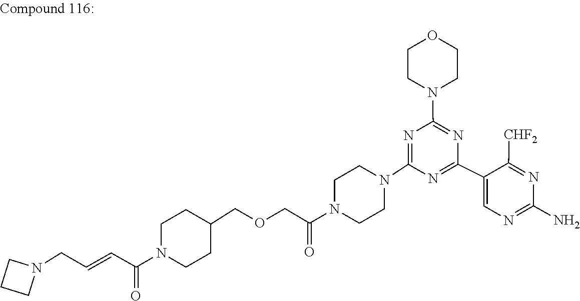 [SMILES: Nc1ncc(-c2nc(N3CCOCC3)nc(N3CCN(C(=O)COCC4CCN(C(=O)/C=C/CN5CCC5)CC4)CC3)n2)c(C(F)F)n1] | (E)-1-(4-((2-(4-(4-(2-amino-4- (difluoromethyl)pyrimidin-5-yl)-6- morpholino-1,3,5-triazin-2-yl)piperazin- 1-yl)-2-oxoethoxy)methyl)piperidin-1- yl)-4-(azetidin-1-yl)but-2-en-1-one |
| 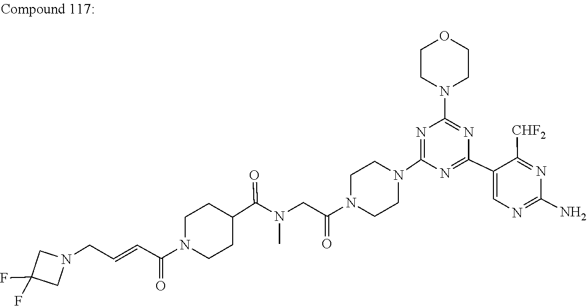 [SMILES: CN(CC(=O)N1CCN(c2nc(-c3cnc(N)nc3C(F)F)nc(N3CCOCC3)n2)CC1)C(=O)C1CCN(C(=O)/C=C/CN2CC(F)(F)C2)CC1] | (E)-N-(2-(4-(4-(2-amino-4- (difluoromethyl)pyrimidin-5-yl)-6- morpholino-1,3,5-triazin-2-yl)piperazin- 1-yl)-2-oxoethyl)-1-(4-(3,3- difluoroazetidin-1-yl)but-2-enoyl)-N- methylpiperidine-4-carboxamide |
| 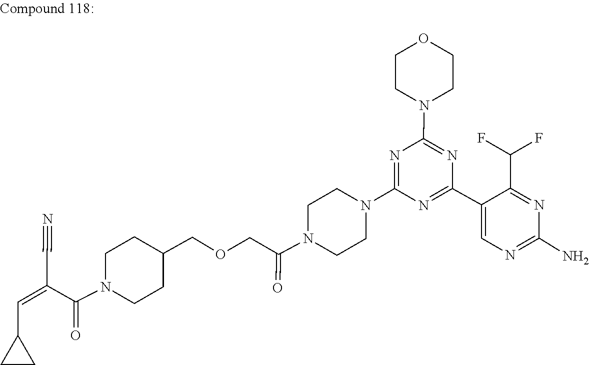 [SMILES: N#C/C(=C/C1CC1)C(=O)N1CCC(COCC(=O)N2CCN(c3nc(-c4cnc(N)nc4C(F)F)nc(N4CCOCC4)n3)CC2)CC1] | (Z)-2-(4-((2-(4-(4-(2-amino-4- (difluoromethyl)pyrimidin-5-yl)-6- morpholino-1,3,5-triazin-2-yl)piperazin- 1-yl)-2-oxoethoxy)methyl)piperidine-1- carbonyl)-3-cyclopropylacrylonitrile |
| 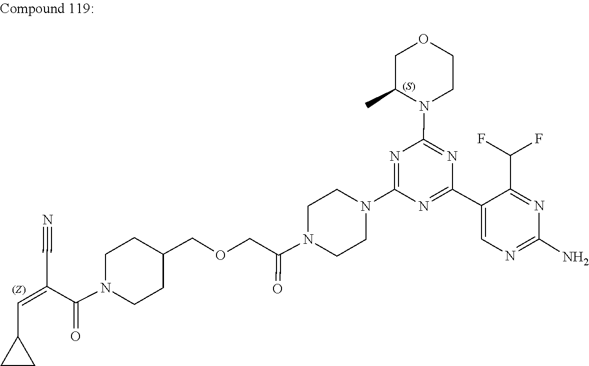 | (S,Z)-2-(4-((2-(4-(4-(2-amino-4- (difluoromethyl)pyrimidin-5-yl)-6-(3- methylmorpholino)-1,3,5-triazin-2- yl)piperazin-1-yl)-2- oxoethoxy)methyl)piperidine-1- carbonyl)-3-cyclopropylacrylonitrile |

Most preferred linkers are the following compounds shown by formula:

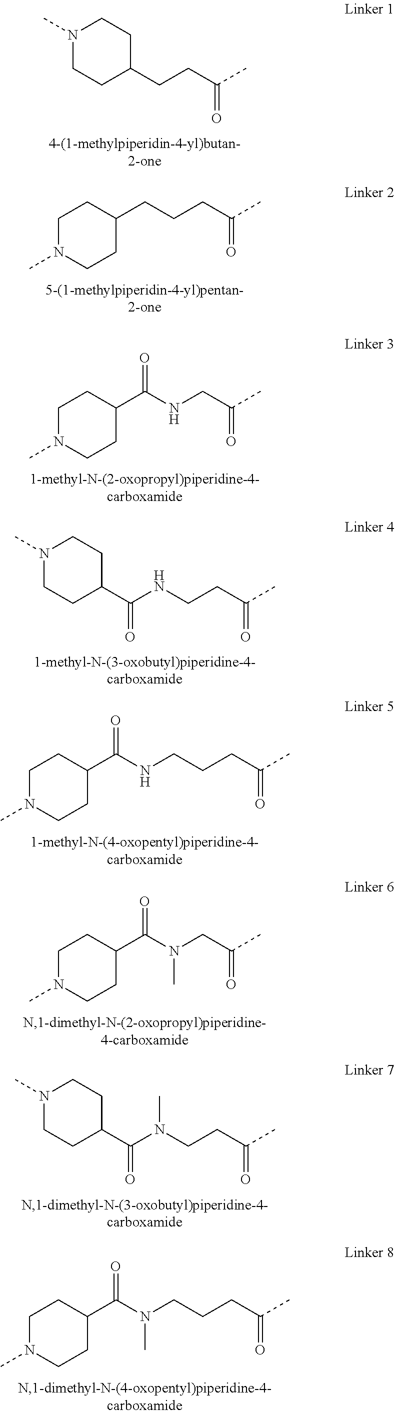

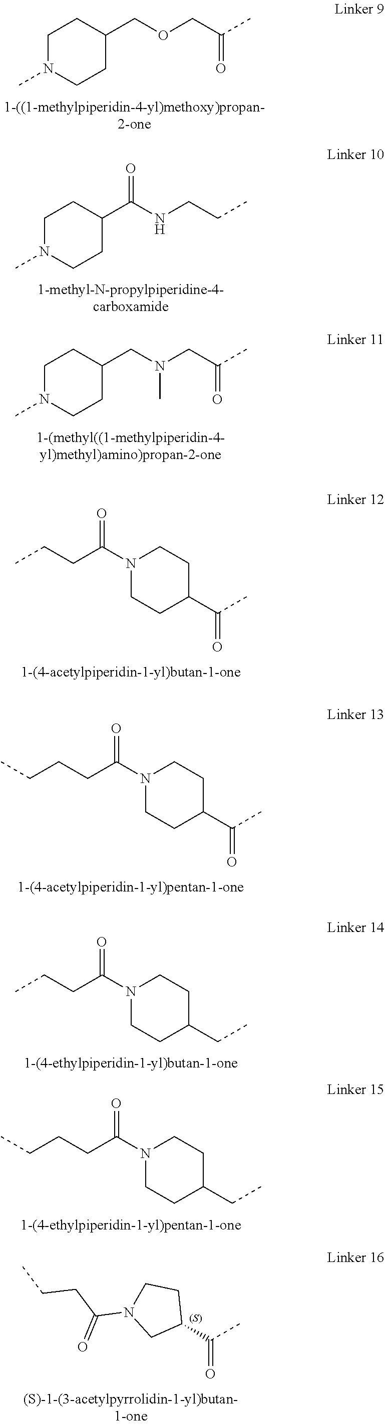

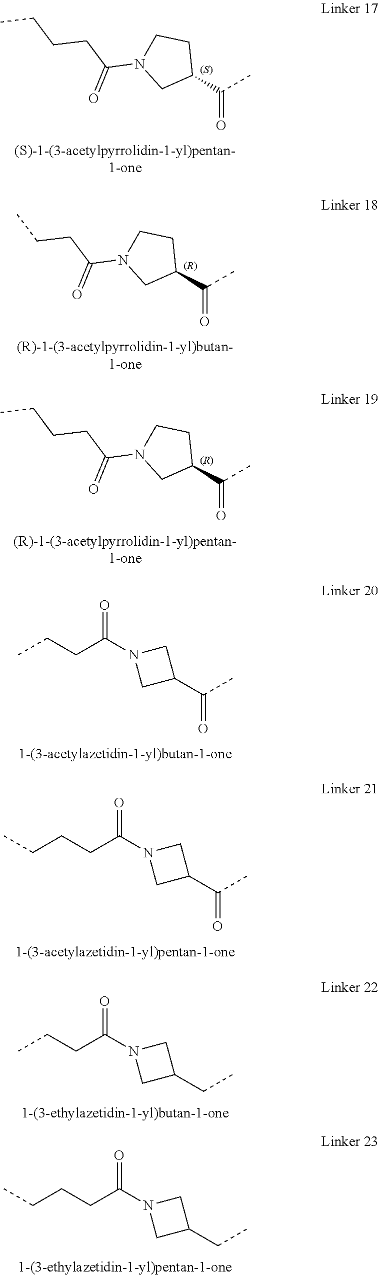

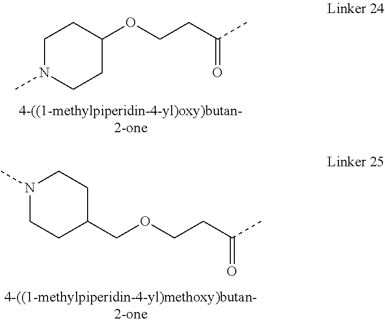

### BRIEF DESCRIPTION OF THE FIGURES

Summary data for some compounds of the invention in comparison to CNX-1351: intrinsic reactivity and in vitro data (Table 1); cellular potency (Table 2); logD and solubility (Table 3) for some of the compounds of the invention and CNX-1351.

FIG. 1 depicts the results of compounds 43 (and 54 (and their reversible analogs 44 (and 55 (in a washout experiment in SKOV3 cells as compared with a known reversible inhibitor (PQR514)²⁴ and CNX-1351²².

FIG. 2. Metabolic stability of 43 and 54 and CNX-1351 using rat liver microsomes fortified with Phase I metabolism cofactor NADPH.

FIG. 3 shows on the left-hand side a model for cellular on- and off target reactions of covalent inhibitors. In the on-target reaction, enzyme E and inhibitor I form first a reversible E˜I complex (equilibrium dependent on Kᵢ), which is then converted to a covalent EI complex (controlled by k_{inact}). Competing off-target reactions with cellular sulfhydryls (S) consume the inhibitor and form a stable SI adduct. Right-hand side: On- and off-target covalent reactions of inhibitors as depicted on the left-hand side were modelled using KinTek Global Kinetic Explorer: concentrations were set to 7 mM for intracellular sulfhydryls (for intracellular reduced glutathione (GSH) concentrations see Ref. 80); for the targeted enzyme (PI3Kα) to 10 nM; and for indicated covalent inhibitors to 100 nM. Experimental values for k_{inact} and k_{chem} used for the modelling are listed in Table 1 and FIG. 4.

FIG. 4 shows KinTek modelling for on- and off-target reactivity. (a, c) Modelling drug-target covalent binding between 10 nM enzyme (PI3Ka) and 100 nM of the indicated inhibitors using experimentally determined Kᵢ (reversible binding affinity, nM), and k_{inact} (covalent bond formation rate, s−1) values for each inhibitor. (b, d) Side reactions between 7 mM GSH and the indicated inhibitors modelled using experimentally determined k_{chem} (intrinsic reactivity, nM−1.·s−1). Modelling was performed using KinTek Global Kinetic Explorer using values from Tables 1. kchem could not be determined reliably for compounds 11 and 17 due to a lack of reactivity with 12 M βME, and for 1 due to a too rapidly completed reaction with 1 mM βME preventing reliable data collection. k_{chem} for compound 15 was lower than 1·10⁻⁶ [M⁻¹·s⁻¹], and was thus considered un-reactive. The graphs in FIG. 4a that are formed by circles are as follows: the upper graph relates to compound 13, the lower graph relates to compound 7.

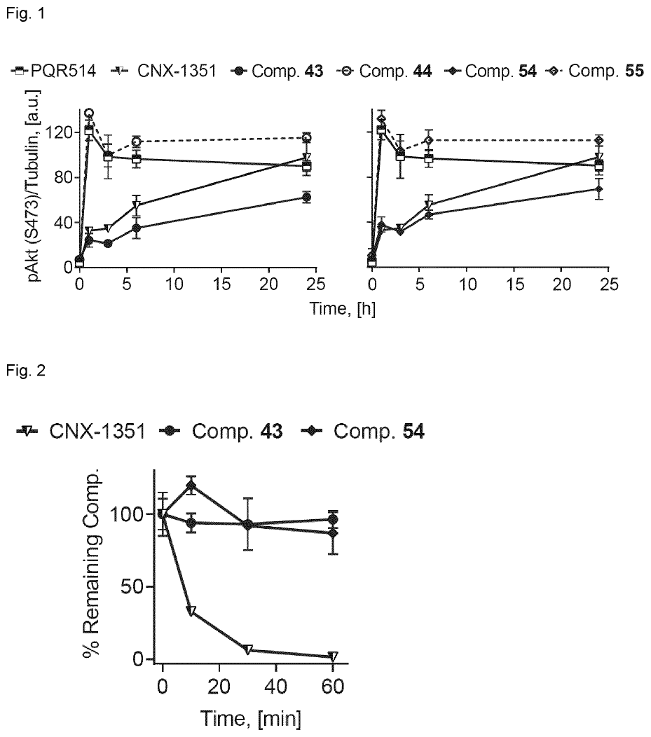

### EXAMPLES

### Preparation of Compounds of the Invention

The compounds of the invention may be synthesized by synthetic routes that include processes analogous to those well known in the chemical arts, particularly in light of the description contained herein. The starting materials are generally available from commercial sources or are readily prepared using methods well known to those skilled in the art.

For illustrative purposes, Schemes 1-9 show general methods for preparing the compounds of the present invention as well as key intermediates. For a more detailed description of the individual reaction steps, see the Examples herein below. Those skilled in the art will appreciate that other synthetic routes may be used to synthesize the compounds of the invention. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In preparing compounds of the invention, protection and deprotection of remote functionality (e.g., primary or secondary amine) of intermediates may be necessary. The need for such protection will vary depending on the nature of the remote functionality and the conditions of the preparation methods. Suitable amino-protecting groups include tert-butyloxycarbonyl (BOC) or N,N-dimethylformamidinyl. The need for such protection is readily determined by one skilled in the art. For a general description of protecting groups and their use, see T. W. Greene, Protective Groups in Organic Synthesis, John Wiley & Sons, New York, 1991.

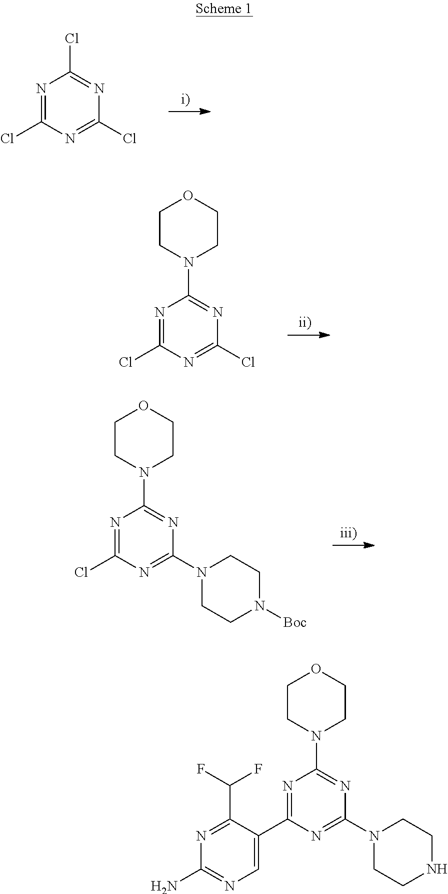

Scheme 1 shows a general method for the preparation of the inhibitors building block using nucleophilic aromatic substitutions and Suzuki coupling. Reagents and conditions: (i) Et₃N, DCM, −50° C., 3 h; (ii) 1-boc-piperazine, DIPEA, EtOH, 0° C.→r.t., 5 h; (iii) tert-Butyl N-[5-bromo-4-(difluoromethyl)pyrimidin-2-yl]-N-[(tert-butoxy)carbonyl]carbamate, bis(pinacolato)diboron, AcOK, Pd(dppf)Cl₂, dioxane, 95° C., 1.5 h, (2) monochloro-triazine, XPhosPdG₂ (cat.), K₃PO₄, H₂O, 100° C., o/n, (3) HCl, dioxane/H₂O, 80° C., o/n.

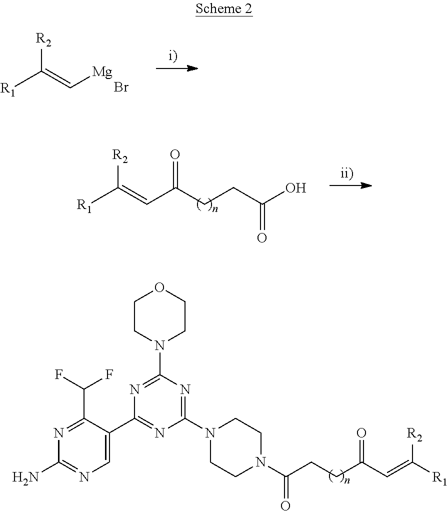

Scheme 2 shows a general method for the preparation of covalent inhibitors of formula (I) having enones as warheads. R₁, R₂ are, independently from each other, H or CH₃; n is 1 or 2 or 3 or 4 or 5. Reactions of cyclic anhydrides with Grignard reagents lead to ring-opened keto acids. The final compounds are obtained by coupling of keto acids with the amine building block reported in Scheme 1. Reagents and conditions: (i) anhydride derivative, THF or DCM, Grignard reagent (0.5 M solution in THF), −78° C., 2-4 h; (ii) keto acid derivative, HCTU, DIPEA, DMF, 0° C.→r.t., 4-16 h.

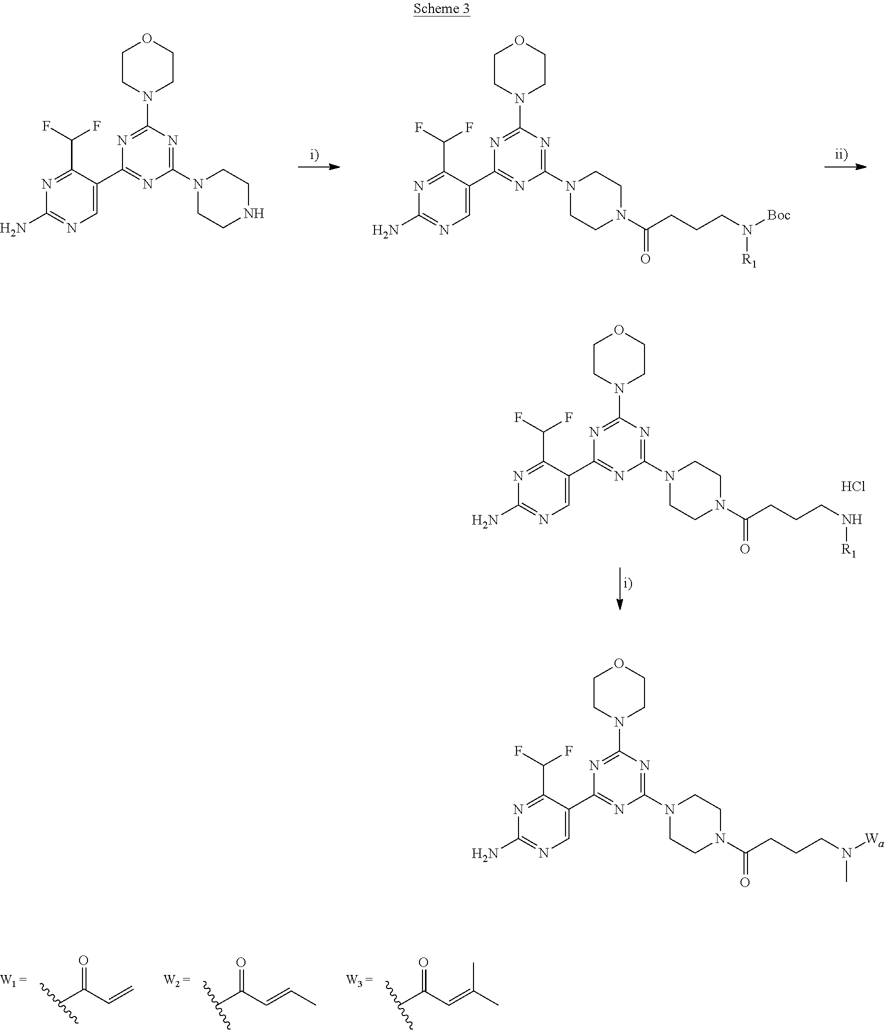

Scheme 3 shows a general method for the preparation of covalent inhibitors of formula (I) having acrylamides as warheads. R₁ is H or CH₃; W stands for warhead. Reagents and conditions: (i) building block or HCl salt, HCTU, DIPEA, DMF, 0° C.→r.t., 4-16 h; (ii) HCl in dioxane (4 M), THF, r.t., 3-16 h.

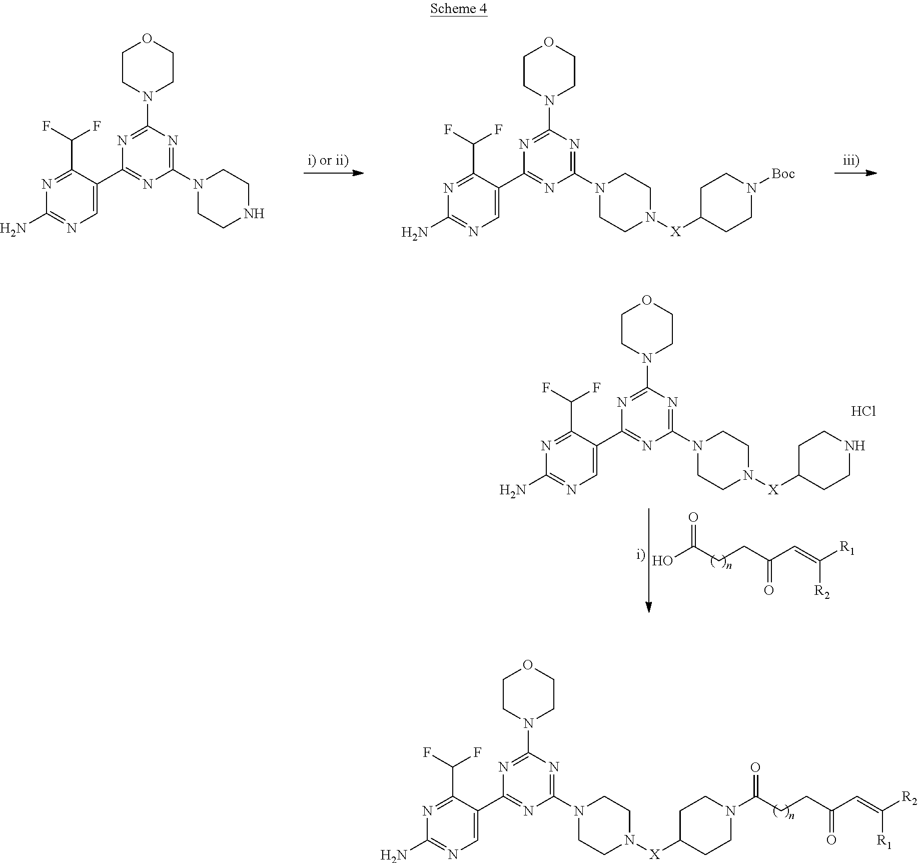

Scheme 4 shows a general method for the preparation of covalent inhibitors of formula (II) having enones as warheads. R₁, R₂ are, independently from each other, H or CH₃; n is 1 or 2. If X is CO: the compounds are synthesized exploiting a sequence of coupling reactions with Boc-protected acid derivatives followed by Boc-deprotection using HCl in dioxane. Reagents and conditions: (i) building block or HCl salt, HCTU, DIPEA, DMF, 0° C.→r.t., 4-16 h; (iii) HCl in dioxane (4 M), THF, r.t., 3-16 h. If X is CH₂: first a nucleophilic substitution takes places, then after Boc-deprotection using HCl in dioxane, the warhead is introduced using a coupling reaction. Reagents and conditions: (ii) building block, K₂CO₃, DMF, 80° C., 5-16 h; (iii) HCl in dioxane (4 M), THF, r.t., 3-16 h; (i) HCl salt, HCTU, DIPEA, DMF, 0° C.→r.t., 4-16 h. The keto acids are synthesized as reported in Scheme 2 by reacting a of cyclic anhydride with a Grignard reagent.

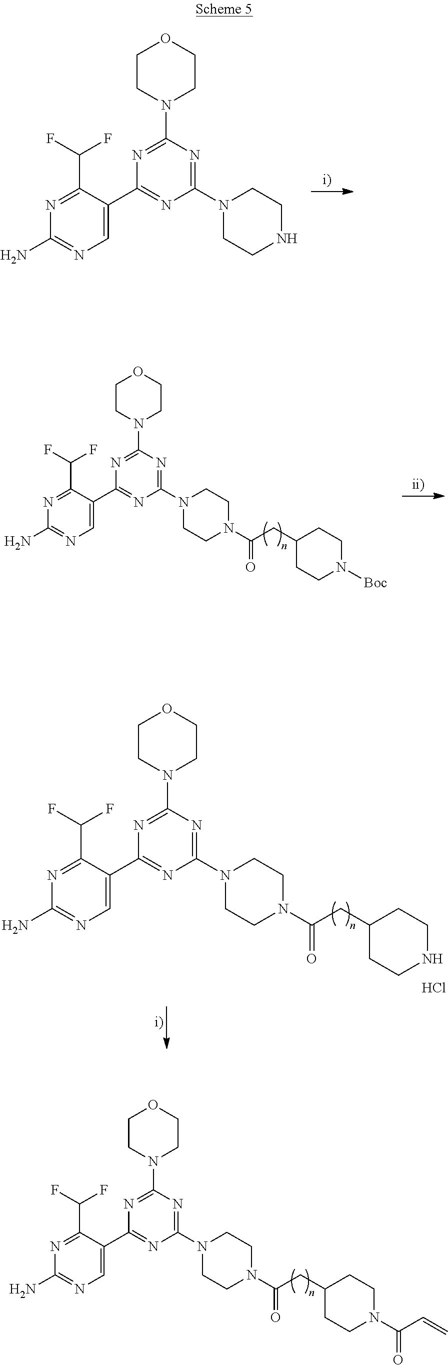

Scheme 5 shows a general method for the preparation of covalent inhibitors of formula (III) having a carbon spacer. n is equal to 0 or 1 or 2 or 3. Reagents and conditions: (i) building block or HCl salt, HCTU, DIPEA, DMF, 0° C.→r.t., 4-16 h; (ii) HCl in dioxane (4 M), THF, r.t., 3-16 h.

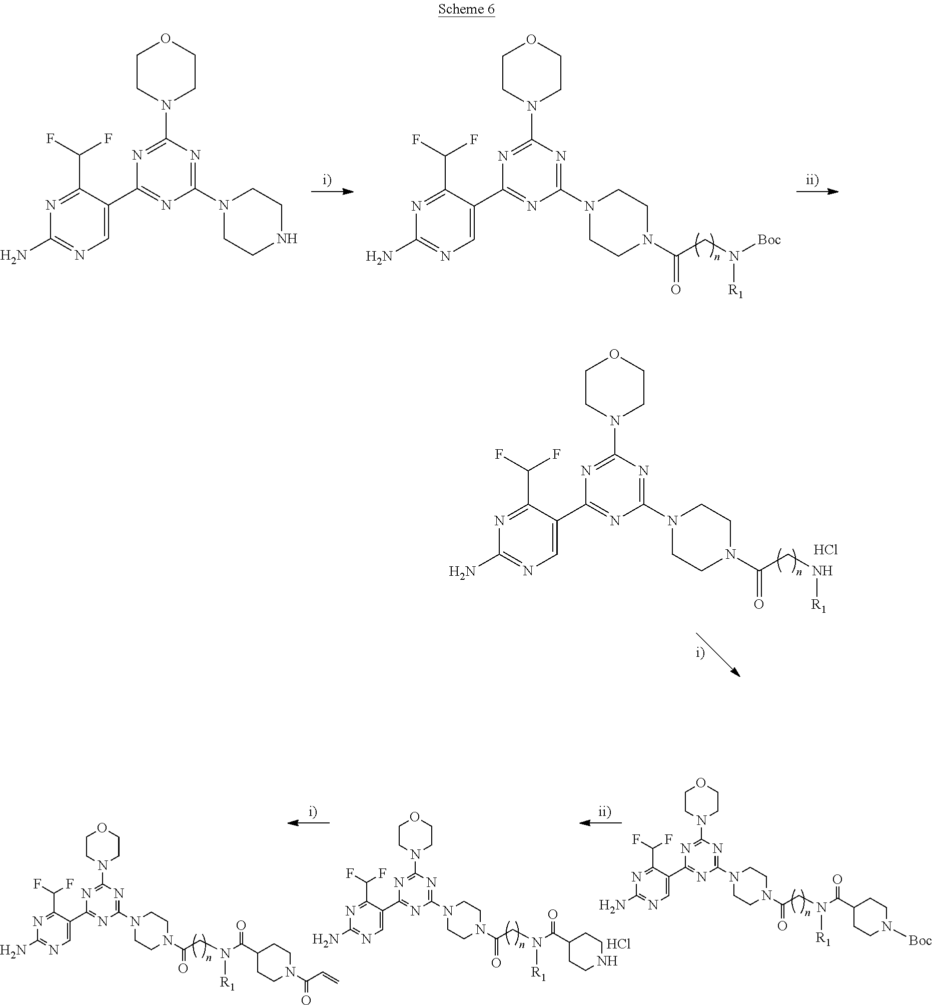

Scheme 6 shows a general method for the preparation of covalent inhibitors of formula (III) having an amide spacer. R₁ is H or CHs; n is equal to 1 or 2 or 3. Reagents and conditions: (i) building block or HCl salt, HCTU, DIPEA, DMF, 0° C.→r.t., 4-16 h; (ii) HCl in dioxane (4 M), THF, r.t., 3-16 h.

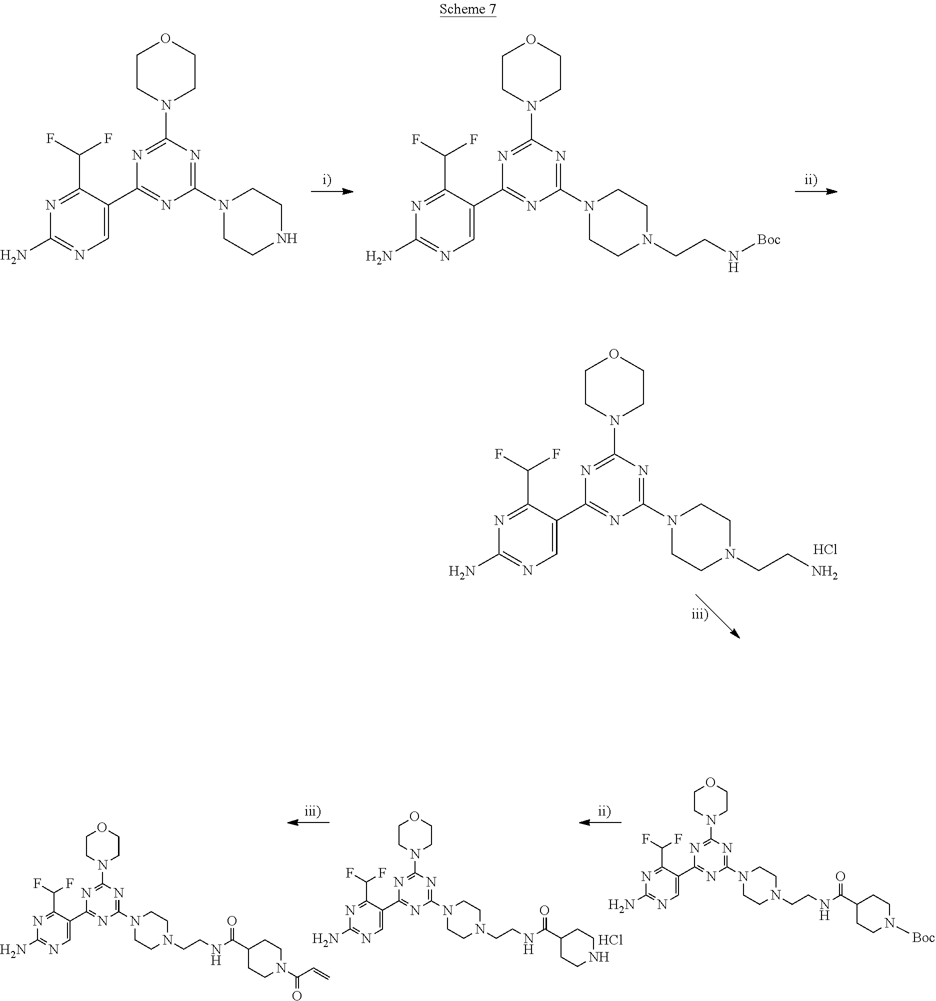

Scheme 7 shows the method for the preparation of compound 62 of formula (III). Reagents and conditions: (1) building block, K₂CO₃, DMF, 80° C., 5-16 h; (ii) HCl in dioxane (4 M), THF, r.t., 3-16 h; (iii) HCl salt, HCTU, DIPEA, DMF, 0° ° C.→r.t., 4-16 h.

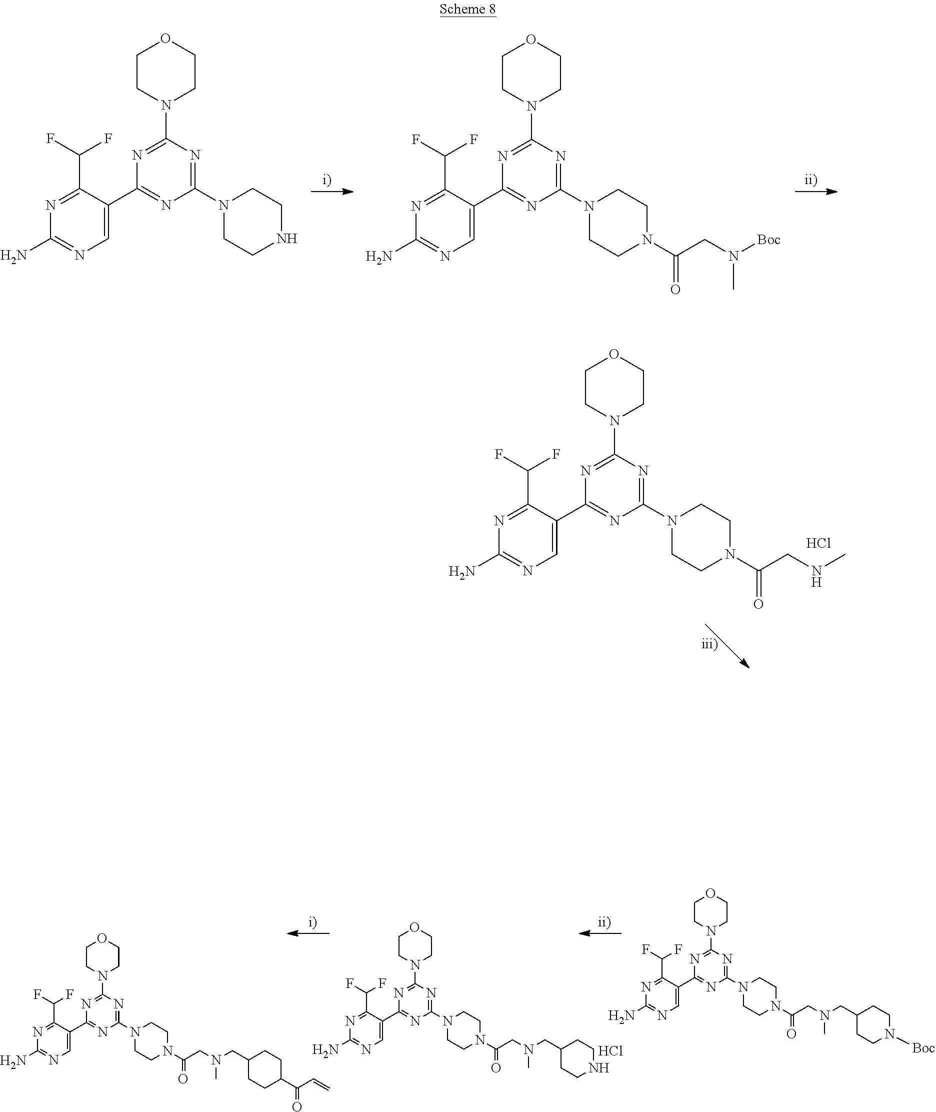

Scheme 8 shows the method for the preparation of compound 63 of formula (III). Reagents and conditions: (i) building block or HCl salt, HCTU, DIPEA, DMF, 0° C.→r.t., 4-16 h.; (ii) HCl in dioxane (4 M), THF, r.t., 3-16 h; (iii) HCl salt, K₂CO₃, DMF, 80° C., 5-16 h.

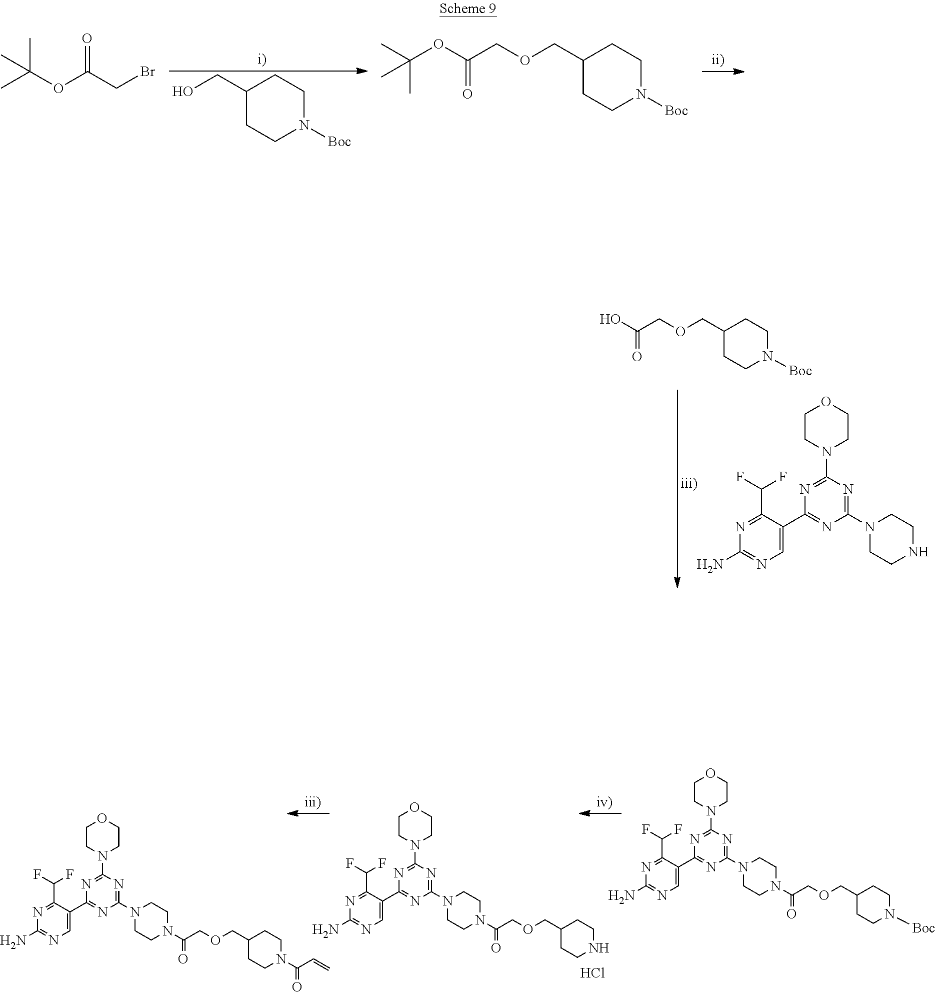

Scheme 9 shows the method for the preparation of compound 61 of formula (III). First the acid bearing an ether in the linker is synthesized by a nucleophilic substitution followed by a hydrolysis of the ester using a strong base in water/THF. Reagents and conditions: (i) NaH (60% dispersion in mineral oil), DMF, 0° C., 15 min; (ii) LiOH (5M in H₂O), THF; (iii) building block or HCl salt, HCTU, DIPEA, DMF, 0° C.→r.t., 2 h; (iv) HCl in dioxane (4 M), THF, r.t., o/n.

### Methods of Separation

In the methods of preparing the compounds of this invention, it may be advantageous to separate reaction products from one another and/or from starting materials. The desired products of each step or series of steps are separated and/or purified to the desired degree of homogeneity by the techniques common in the art. Typically such separations involve extraction, crystallization from a solvent or solvent mixture, or chromatography. Chromatography can involve any number of methods including, for example: reverse-phase and normal phase; high, medium and low-pressure liquid chromatography methods and apparatus; small scale analytical; and preparative thin or thick layer chromatography, as well as techniques of small scale thin layer and flash chromatography.

Selection of appropriate methods of separation depends on the nature of the materials involved, for example, presence or absence of polar functional groups in chromatography, stability of materials in acidic and basic media in multiphase extraction. One skilled in the art will apply techniques most likely to achieve the desired separation.

### EXAMPLES

The Examples are intended to illustrate the present invention without restricting it.

The chemical reactions described in the Examples may be readily adapted to prepare a number of other inhibitors of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

Reagents were purchased at the highest commercial quality from Acros Organics, Sigma-Aldrich, Apollo Scientific or Fluorochem and used without further purification. Solvents were purchased from Acros Organics in AcroSeal® bottles over molecular sieves. Grignard reactions, cross-coupling reactions and peptide-coupling reactions were carried out under nitrogen atmosphere in anhydrous solvents, and glassware was oven dried prior to use. Thin layer chromatography (TLC) plates were purchased from Merck KGAA (Polygram SIL/UV254, 0.2 mm silica with fluorescence indicator) and UV light (254 nm) was used to visualize the compounds. Flash chromatography was performed with Isco CombiFlash Companion systems using prepacked silica gel columns (40-60 μm particle size RediSep). ¹H, ¹⁹F and ¹³C NMR spectra were recorded on a Bruker Avance 400 spectrometer. NMR spectra were obtained in deuterated solvents, namely CDCl₃ or (CD₃)₂SO. The chemical shift (δ values) are reported in ppm and corrected to the signal of the deuterated solvents (7.26 ppm (¹H NMR) and 77.16 ppm (¹³C NMR) for CDCl₃; and 2.50 ppm (¹H NMR) and 39.52 ppm (¹³C NMR) for (CD₃)₂SO. ¹⁹F NMR spectra are calibrated relative to CFCl₃ (δ=0 ppm) as external standard. When peak multiplicities are reported, the following abbreviations are used: s (singlet), d (doublet), dd (doublet of doublets), t (triplet), td (triplet of doublets), q (quartet), doublet of quartets (dq), m (multiplet), br (broadened signal). Coupling constants, when given, are reported in Hertz (Hz). High resolution mass spectra (HRMS) were recorded on a Thermo Fisher Scientific LTQ Orbitrap XL (nanoESI-MS) spectrometer. MALDI-TOF mass spectra were obtained on a Voyager-De™ Pro measured in m/z. The chromatographic purity of final compounds was determined by high performance liquid chromatography (HPLC) analyses on an Ultimate 3000SD System from ThermoFisher with LPG-3400SD pump system, ACC-3000 autosampler and column oven, and DAD-3000 diode array detector. An Acclaim-120 C18 reversed-phase column from ThermoFisher was used as stationary phase. Gradient elution (5:95 for 0.2 min, 5:95→100:0 over 10 min, 100:0 for 3 min) of the mobile phase consisting of CH₃CN/MeOH:H₂O_{(10.90)} was used at a flow rate of 0.5 mL/min at 40° C. The purity of all final compounds was higher than 95%.

The following abbreviations are used hereinafter: DMSO (dimethyl sulfoxide), HCl (hydrochloric acid), M (molar), MALDI (Matrix-assisted Laser Desorption/lonization), HRMS (High resolution mass spectra), MS (mass spectrometry), PBS (phosphate buffered saline), TLC (thin layer chromatography).

### Preparation of Intermediate Compounds

The following methods were used to prepare the intermediates compounds used to produce compounds of formula (I, II, III, IV and V).

### Method 1: tert-butyl 4-(4-chloro-6-morpholino-1,3,5-triazin-2-yl)piperazine-1-carboxylate (I1)

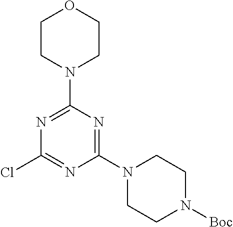 [SMILES: CC(C)(C)OC(=O)N1CCN(c2nc(Cl)nc(N3CCOCC3)n2)CC1]

To a solution of 2,4-dichloro-6-(morpholin-4-yl)-1,3,5-triazine (20.7 g, 87.9 mmol, 0.9 equiv) in ethanol, DIPEA (16.2 g, 21.4 ml, 125.6 mmol, 1.3 equiv) and 1-Boc-piperazine (18.0 g, 96.6 mmol, 1.0 equiv) were added at 0° C. The reaction mixture was stirred at room temperature for 5 hours. The solvent was removed under reduced pressure. Dichloromethane (300 mL) was added and the resulting organic layer was washed with an aqueous saturated NaHSO₄-solution (4×200 mL). The organic layer was dried over anhydrous Na₂SO₄, filtered and concentrated under reduced pressure. The product was recrystallized from dichloromethane/heptanes to obtain tert-butyl 4-(4-chloro-6-morpholino-1,3,5-triazin-2-yl)piperazine-1-carboxylate as a colorless solid (22.7 g, 58.9 mmol, 80%). MALDI-MS: m/z=385.643 [M+H]⁺. HPLC: t_{R}=6.53 min (100.0% purity).

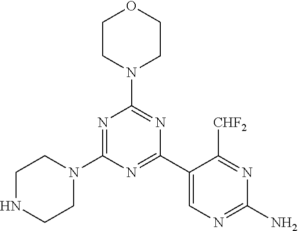 [SMILES: Nc1ncc(-c2nc(N3CCNCC3)nc(N3CCOCC3)n2)c(C(F)F)n1]

### Method 2: 4-(difluoromethyl)-5-(4-morpholino-6-(piperazin-1-yl)-1,3,5-triazin-2-yl)pyrimidin-2-amine (I2)

Step 1. tert-Butyl N-[5-bromo-4-(difluoromethyl)pyrimidin-2-yl]-N-[(tert-butoxy)carbonyl]carbamate (7.00 g, 16.50 mmol, 1.0 equiv), Bis(pinacolato)diboron (6.29 g, 24.75 mmol, 1.5 equiv), potassium acetate (5.02 g, 51.15 mmol, 3.1 equiv), [1,1′-bis(diphenylphosphino)ferrocene]dichloropalladium(II) (Pd(dppf)Cl₂, 1.21 g, 1.65 mmol, 0.1 equiv) were charged in flask under nitrogen atmosphere. Absolute 1,4-dioxane (40 mL) was added and the mixture was stirred at 95° C. for 1.5 hour. After completion of the reaction, the mixture was allowed to cool down to room temperature. Step 2. Monochloro-triazine 4-(4-chloro-6-morpholino-1,3,5-triazin-2-yl)piperazine-1-carboxylate (6.99 g, 18.15 mmol, 1.1 equiv), chloro(2-dicyclohexylphosphino-2′,4′,6′-triisopropyl-1,1′-biphenyl)[2-(2′-amino-1,1′-biphenyl)]palladium(II) (XPhos Pd G2, 0.519 g, 0.66 mmol, 0.04 equiv), potassium phosphate tribasic (10.51 g, 49.50 mmol, 3.0 equiv) and deionized H₂O (10 mL) were added. The resulting mixture was stirred at 100° C. overnight. After completion of the reaction, the mixture was allowed to cool down to room temperature and the crude was filtered on celite. To the solution, deionized H₂O (300 mL) and ethyl acetate (300 mL) were added. The layers were separated and the organic layer was washed with deionized H₂O and brine (2×). The aqueous layer was extracted with dichloromethane (2×). The combined organic layers were concentrated under reduced pressure. Step 3. The above residue was dissolved in 1,4-dioxane (40 mL) and an aqueous solution of HCl (3 M, 40 mL) was added. The reaction mixture was stirred at 80° C. overnight. After completion of the reaction, the mixture was cool down to room temperature. Ethyl acetate (200 mL) and deionized H₂O (200 mL) were added and the two layers were separated. The aqueous layer was washed with ethyl acetate (3×). The aqueous layer was basified to pH=10. The solid formed was filtered and washed with acetonitrile to obtain 4-(difluoromethyl)-5-(4-morpholino-6-(piperazin-1-yl)-1,3,5-triazin-2-yl)pyrimidin-2-amine as a beige solid (4.91 g, 12.48 mmol, 76%). MALDI-MS: m/z=394.205 [M+H]⁺.

### Method 3: (E)-4-oxohept-5-enoic acid (I3)

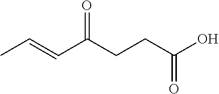

To a solution of succinic anhydride (500 mg, 5.00 mmol, 1.0 equiv) in anhydrous tetrahydrofuran (THF, approx. 1 mL/0.25 mmol) at −78° C. under nitrogen atmosphere, 1-propenylmagnesium bromide (0.5 M solution in THF, 8.99 mmol, 18.0 ml, 1.8 equiv) was added dropwise. The reaction was stirred at −78° C. for 2 hours. A 1 N aqueous HCl-solution (approx. 1 mL / 0.55 mmol) was added and the mixture was slowly warmed up to room temperature. The pH was adjusted to ˜2 by 1 N aqueous HCl-solution. Tetrahydrofuran was removed under reduced pressure and the remaining aqueous layer was extracted with dichloromethane (3×). The combined organic layers were dried over Na₂SO₄, filtered and concentrated under reduced pressure. (E)-4-oxohept-5-enoic acid was obtained as a yellowish oil (512 mg, 3.60 mmol, 72%) and used for the next step without further purification. ¹H NMR (400 MHZ, CDCl₃): δ6.91 (dq, ³J_{H,H}=15.8 Hz, ³J_{H,H}=6.8 Hz, 1 H), 6.16 (dd, ³J_{H,H}=15.8 Hz, ⁴J_{H,H}=1.7 Hz, 1 H), 2.88 (t, ³J_{H,H}=6.6 Hz, 2 H), 2.67 (t, ³J_{H,H}=6.6 Hz, 2 H), 1.92 (dd, ³J_{H,H}=6.8 Hz, ⁴J_{H,H}=1.7 Hz, 3 H).

### Method 4: tert-butyl 4-(4-(4-(2-amino-4-(difluoromethyl)pyrimidin-5-yl)-6-morpholino-1,3,5-triazin-2-yl)piperazine-1-carbonyl)piperidine-1-carboxylate (I4)

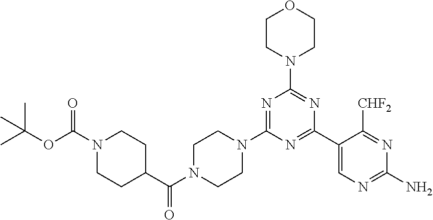 [SMILES: CC(C)(C)OC(=O)N1CCC(C(=O)N2CCN(c3nc(-c4cnc(N)nc4C(F)F)nc(N4CCOCC4)n3)CC2)CC1]

To a solution of 1-(tert-Butoxycarbonyl)piperidine-4-carboxylic acid (513 mg, 2.24 mmol, 1.1 equiv) in anhydrous N,N-dimethylformamide (DMF, approx. 1 mL/0.18 mmol), O-(1H-6-chlorobenzotriazole-1-yl)-1,1,3,3-tetramethyluronium hexafluorophosphate (HCTU, 1.1 equiv) and N,N-diisopropylethylamine (3.2 equiv) were added at 0° ° C. under nitrogen atmosphere. The resulting mixture was stirred for 5 minutes at 0° C., then 4-(difluoromethyl)-5-(4-morpholino-6-(piperazin-1-yl)-1,3,5-triazin-2-yl)pyrimidin -2-amine (or the respective HCl salt) was added and the reaction was stirred at room temperature for 4-16 hours. After completion of the reaction, DMF was removed under high vacuum. The crude was dissolved in DCM and the organic layer was washed with deionized H₂O (2×) and an aqueous saturated Na₂CO₃-solution (3×). The organic layer was dried over Na₂SO₄, filtered and concentrated under reduced pressure. Purification by column chromatography on silica gel (dichloromethane/methanol/ammonia: 100:0:0→96:4:0.04) gave compound tert-butyl 4-(4-(4-(2-amino-4-(difluoromethyl)pyrimidin -5-yl)-6-morpholino-1,3,5-triazin-2-yl)piperazine-1-carbonyl)piperidine-1-carboxylate as a colorless solid (1.061 g, 1.75 mmol, 86%). MALDI-MS: m/z=605.893 [M+H]⁺; m/z=549.645 [(M−t-Butyl)+H]⁺.

### Method 5: 2-(4-(4-(2-amino-4-(difluoromethyl)pyrimidin-5-yl)-6-morpholino-1,3,5-triazin-2-yl)piperazin-1-yl)-2-oxoethan-1-aminium chloride (I5)

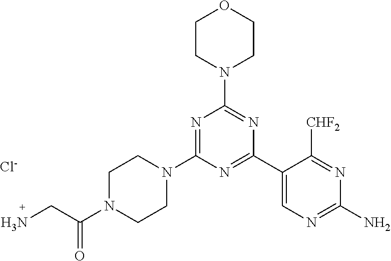 [SMILES: [Cl-] ; NCC(=O)N1CCN(c2nc(-c3cnc(N)nc3C(F)F)nc(N3CCOCC3)n2)CC1]

To a solution of the respective Boc-protected amine (1.0 equiv.) in THF (approx. 1 mL/0.10 mmol), a 4 M solution of HCl in dioxane (17 equiv.) was added dropwise. The mixture was stirred at room temperature for 16 hours. The mixture was reduced to dryness under reduced pressure. The product was precipitated from ACN, filtered and washed with cold ACN. The HCl salt of the product was used for the next step without further purification. 4-(4-(4-(2-Amino-4-(difluoromethyl)pyrimidin-5-yl)-6-morpholino-1,3,5-triazin-2-yl)piperazin-1-yl)-N-methyl-4-oxobutan-1-aminium chloride was obtained as a colorless solid (1.059 mg, 2.18 mmol, quantitative). MALDI-MS: m/z=451.140 [M+H]⁺.

### Method 6: tert-butyl 3-((4-(4-(2-amino-4-(difluoromethyl)pyrimidin-5-yl)-6-morpholino-1,3,5-triazin-2-yl)piperazin-1-yl)methyl)azetidine-1-carboxylate (I6)

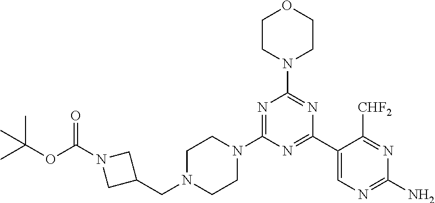 [SMILES: CC(C)(C)OC(=O)N1CC(CN2CCN(c3nc(-c4cnc(N)nc4C(F)F)nc(N4CCOCC4)n3)CC2)C1]

To a suspension of 4-(difluoromethyl)-5-(4-morpholino-6-(piperazin-1-yl)-1,3,5-triazin-2-yl)pyrimidin-2-amine in N,N-dimethylformamide (5 ml), N,N-diisopropylethylamine (1.05 g, 1.4 mL, 8.12 mmol, 3.2 equiv) and 1-boc-3-(bromomethyl)azetidine (521 mg, 2.54 mmol, 1 equiv) were added under nitrogen atmosphere. The resulting mixture was stirred at 90° C. overnight. After completion of the reaction, N,N-dimethylformamide was removed under high vacuum. The crude was dissolved in dichloromethane and the organic layer was washed with deionized water (2×) and an aqueous saturated Na₂CO₃-solution (3×). The organic layer was dried over Na₂SO₄, filtered and concentrated under reduced pressure. Purification by column chromatography on silica gel (dichloromethane/methanol/ammonia: 100:0:0→96:4:0.04) gave the desired compound as a yellowish solid (940 mg, 1.67 mmol, 66%). MALDI-MS: m/z=563.534 [M+H]⁺.

Method 3 is also used for the preparation of the following intermediate compounds, but not limited to them:

| | Reagent | Structure | NMR |
|---|---|---|---|
| I7 | 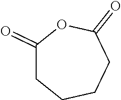 | 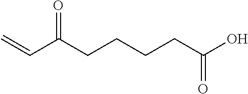 | ¹H NMR (400 MHz, CDCl₃): δ 6.36 (dd, ³J_{H,H} = 17.7 Hz, ³J_{H,H} = 10.5 Hz, 1 H), 6.22 (d, ³J_{H,H} = 17.7 Hz, 1 H), 5.84 (dd, ³J_{H,H} = 10.5 Hz, ²J_{H,H} = 1.4 Hz, 1 H), 2.64-2.61 (m, 2 H), 2.41-2.37 (m, 2 H), 1.74-1.63 (m, 4 H). |
| I8 | 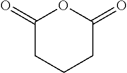 | 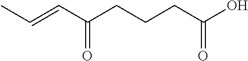 | ¹H NMR (400 MHz, CDCl₃): δ 6.87 (dq, ³J_{H,H} = 15.8 Hz, ³J_{H,H} = 6.8 Hz, 1 H), 6.12 (dq, ³J_{H,H} = 15.8 Hz, ⁴J_{H,H} = 1.7 Hz, 1 H), 2.63 (t, ³J_{H,H} = 7.2 Hz, 2 H), 2.42 (t, ³J_{H,H} = 7.2 Hz, 2 H), 1.96 (q, ³J_{H,H} = 7.1 Hz, 2 H), 1.90 (dd, ³J_{H,H} = 6.8, |
| | | | ⁴J_{H,H} = 1.7 Hz, 3 H). |
| I9 | 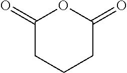 | 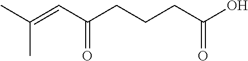 | ¹H NMR (400 MHz, CDCl₃): δ 6.07-6.05 (m, 1 H), 2.50 (t, ³J_{H,H} = 7.2 Hz, 2 H), 2.40 (t, ³J_{H,H} = 7.3 Hz, 2 H), 2.15 (d, ⁴J_{H,H} = 1.3 Hz, 3 H), 1.96-1.90 (m, 2 H), 1.89 (d, ⁴J_{H,H} = 1.3 Hz, 3 H). |

Method 4 is also used for the preparation of the following intermediate compounds, but not limited to them:

| | Reagent | Structure |
|---|---|---|
| I10 | 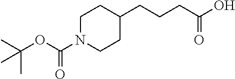 [SMILES: CC(C)(C)OC(=O)N1CCC(CCCC(=O)O)CC1] | 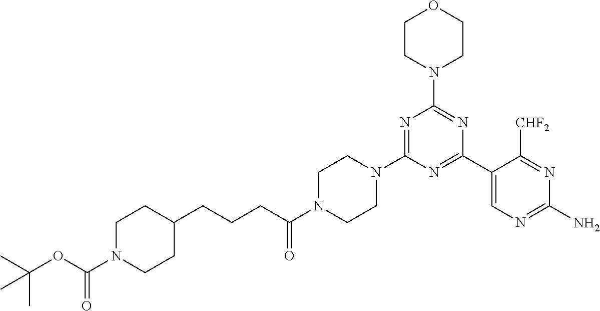 [SMILES: CC(C)(C)OC(=O)N1CCC(CCCC(=O)N2CCN(c3nc(-c4cnc(N)nc4C(F)F)nc(N4CCOCC4)n3)CC2)CC1] |
| | 45¹H NMR (400 MHz, CDCl₃): δ 9.23 (s, 1 H), 7.60 (t, ²J_{H,F} = 54.4 Hz, 1 H), 5.66 (br s, 2 H), | |
| | 4.12-4.03 (m, 2 H), 3.92-3.80 (m, 8 H), 3.78-3.73 (m, 4 H), 3.72-3.68 (m, 2 H), 3.56-3.51 (m, | |
| | 2 H), 2.72-2.63 (m, 2 H), 2.36 (t, ³J_{H,H} = 7.5 Hz, 2 H), 1.72-1.63 (m, 4 H), 1.45 (s, 9 H), 1.48- | |
| | 1.38 (m, 1 H), 1.33-1.25 (m, 2 H), 1.14-1.03 (m, 2 H). | |
| | MALDI-MS: m/z = 647.767 [M + H]⁺, 591.531 [M − tert-butyl + H]⁺. | |
| I11 | 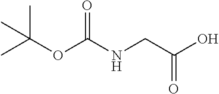 | 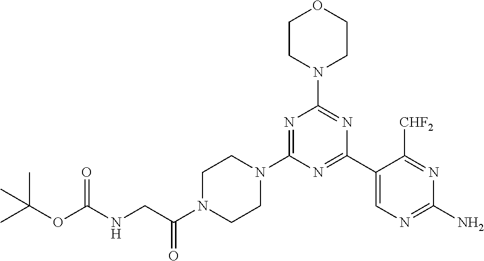 [SMILES: CC(C)(C)OC(=O)NCC(=O)N1CCN(c2nc(-c3cnc(N)nc3C(F)F)nc(N3CCOCC3)n2)CC1] |
| | ¹H NMR (400 MHz, CDCl₃): δ 9.22 (s, 1 H), 7.58 (t, ²J_{H,F} = 54.4 Hz, 1 H), 5.62 (br s, 2 H), 5.51 | |
| | (br s, 1 H), 4.06-3.97 (m, 2 H), 3.97-3.61 (m, 14 H), 3.50-3.42 (m, 2 H), 1.46 (s, 9 H). | |
| | MALDI-MS: m/z = 495.417 [M − Boc + H]⁺. | |
| I12 | 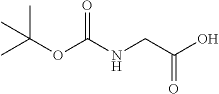 | 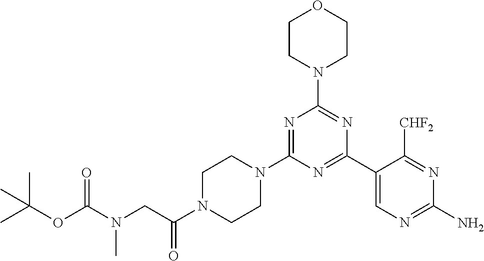 [SMILES: CN(CC(=O)N1CCN(c2nc(-c3cnc(N)nc3C(F)F)nc(N3CCOCC3)n2)CC1)C(=O)OC(C)(C)C] |
| | ¹H NMR (400 MHz, CDCl₃): δ 9.22 (s, 1 H), 7.59 (t, ²J_{H,F} = 54.4 Hz, 1 H), 5.60 (br s, 2 H), 4.15- | |
| | 4.09 (m, 2 H), 4.03-3.80 (m, 8 H), 3.78-3.73 (m, 4 H), 3.73-3.66 (m, 2 H), 3.56-3.48 (m, 2 H), | |
| | 2.05 (s, 3 H), 1.48 (s, 9 H). | |
| | MALDI-MS: m/z = 565.111 [M + H]⁺. | |
| I13 | 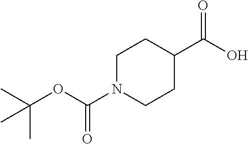 [SMILES: CC(C)(C)OC(=O)N1CCC(C(=O)O)CC1] | 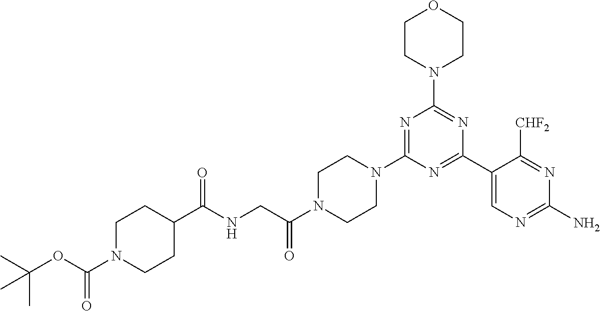 [SMILES: CC(C)(C)OC(=O)N1CCC(C(=O)NCC(=O)N2CCN(c3nc(-c4cnc(N)nc4C(F)F)nc(N4CCOCC4)n3)CC2)CC1] |
| | ¹H NMR (400 MHz, CDCl₃): δ 9.22 (s, 1 H), 7.58 (t, ²J_{H,F} = 54.4 Hz, 1 H), 6.63 (br s, 1 H), 5.62 | |
| | (br s, 2 H), 4.21-4.09 (m, 2 H), 4.11 (d, ³J_{H,H} = 4.0 Hz, 2 H), 3.96-3.80 (m, 8 H), 3.79-3.70 (m, | |
| | 6 H), 3.49 (t, ³J_{H,H} = 5.2 Hz, 2 H), 2.80-2.71 (m, 2 H), 2.38-2.30 (m, 1 H), 1.88-1.81 (m, 2 H), | |
| | 1.72-1.63 (m, 2 H), 1.46 (s, 9 H). | |
| | MALDI-MS: m/z = 662.366 [M + H]⁺. | |
| I14 | 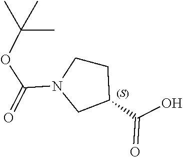 | 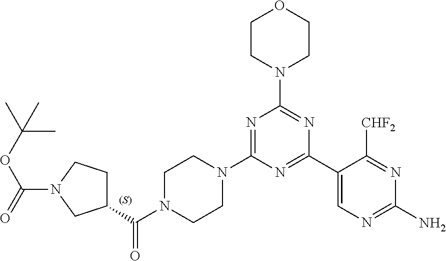 |

¹H NMR (400 MHz, CDCl₃): δ 9.16 (s, 1 H), 7.52 (t, ²J_{H,F} = 54.4 Hz, 1 H), 5.56 (br s, 2 H), 3.92-
3.73 (m, 8 H), 3.70-3.67 (m, 7 H), 3.59-3.40 (m, 4 H), 3.35-3.25 (m, 1 H), 3.22-3.12 (m, 1 H),
2.28-1.93 (m, 2 H), 1.39 (s, 9 H). The compound exists as a mixture of rotamers.
MALDI-MS: m/z = 591.508 [M + H]⁺; m/z = 535.327 [(M − t-Butyl) + H]⁺.

Method 5 is also used for the preparation of the following intermediate compounds, but not limited to them:

| | Structure | NMR | MS |
|---|---|---|---|
| I15 | 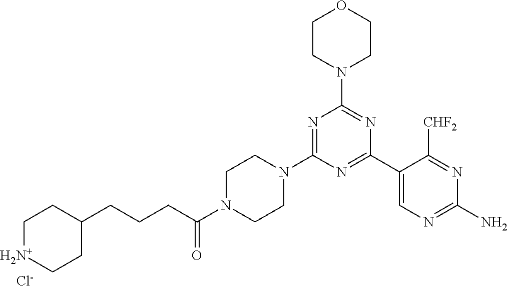 [SMILES: Nc1ncc(-c2nc(N3CCOCC3)nc(N3CCN(C(=O)CCCC4CC[NH2+]CC4)CC3)n2)c(C(F)F)n1 ; [Cl-]] | ¹H NMR (400 MHz, DMSO- d₆): δ 9.11 (s, 1 H), 8.97 (br s, 1 H), 8.73 (br s, 1 H), 7.64 (t, ²J_{H,F} = 54.0 Hz, 1 H), 7.65 (br s, 2 H), 3.86-3.71 (m, 7 H), 3.69-3.61 (m, 4 H), 3.56-3.49 (m, 4 H), 3.21 (d, ³J_{H,H} = 12.5 Hz, 2 H), 2.80 (q, ³J_{H,H} = 12.0 Hz, 2 H), 2.35 (t, ³J_{H,H} = 7.4 Hz, 2 H), 1.82-1.72 (m, 3 H), 1.57-1.47 (m, 3 H), 1.36-1.21 (m, 4 H). | MALDI-MS: m/z = 547.389 [M + H]⁺. |
| I16 | 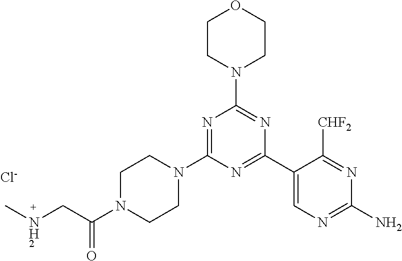 [SMILES: CNCC(=O)N1CCN(c2nc(-c3cnc(N)nc3C(F)F)nc(N3CCOCC3)n2)CC1 ; [Cl-]] | ¹H NMR (400 MHz, DMSO- d6): δ 9.12 (s, 1 H), 9.07 (br s, 2 H), 7.64 (br s, 2 H), 7.63 (t, ²J_{H,F} = 54.0 Hz, 1 H), 4.10 (t, ³J_{H,H} = 5.9 Hz, 2 H), 3.90-3.74 (m, 8 H), 3.68-3.64 (m, 4 H), 3.62-3.58 (m, 2 H), 3.50-3.45 (m, 2 H), 2.58-2.55 (m, 3 H). | MALDI-MS: m/z = 465.437 [M + H]⁺. |
| I17 | 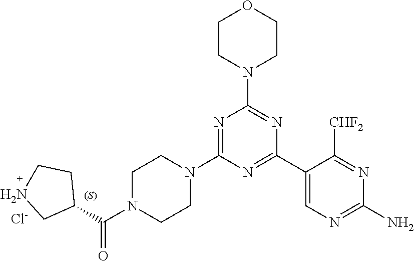 | ¹H NMR (400 MHz, CDCl₃): δ 9.47 (br s, 1 H), 9.17 (br s, 1 H), 9.12 (s, 1 H), 7.64 (t, ²J_{H,F} = 54.0 Hz, 1 H), 3.90-3.74 (m, 8 H), 3.67-3.53 (m, 10 H), 3.43-3.36 (m, 1 H), 3.34-3.28 (m, 1 H), 3.21-3.14 (m, 2 H), 2.26-2.17 (m, 1 H), 1.96-1.90 (m, 1 H). The compound exists as a mixture of rotamers. | MALDI-MS: m/z = 491.753 [M + H]⁺. |

Method 6 is also used for the preparation of the following intermediate compounds, but not limited to them:

| | Reagent | Structure |
|---|---|---|
| 118 | 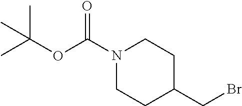 | 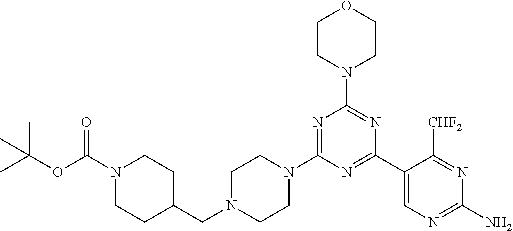 [SMILES: CC(C)(C)OC(=O)N1CCC(CN2CCN(c3nc(-c4cnc(N)nc4C(F)F)nc(N4CCOCC4)n3)CC2)CC1] |

¹H NMR (400 MHz, CDCl₃): δ 9.23 (s, 1 H), 7.63 (t, ²J_{A,F} = 54.4 Hz, 1 H), 5.59 (br s, 2
H), 4.17-4.06 (m, 2 H), 3.91-3.80 (m, 8 H), 3.76-3.74 (m, 4 H), 2.74-2.67 (m, 2 H),
2.44 (t, ³J_{H,H} = 5.1 Hz, 4 H), 2.21 (d, ³J_{H,H} = 7.1 Hz, 2 H), 1.77-1.74 (m, 2 H), 1.70-1.65
(m, 1 H), 1.46 (s, 9H), 1.15-1.05 (m, 2 H).
MALDI-MS: m/z = 591.531 [M + H]⁺.

### Preparation of Compounds of the Invention

### General Procedure 1:

To a solution of the respective carboxylic acid (1.0-1.5 equiv) in anhydrous N,N-dimethylformamide (DMF, approx. 1 mL/0.18 mmol), O-(1H-6-chlorobenzotriazole-1-yl)-1,1,3,3-tetramethyluronium hexafluorophosphate (HCTU, 1.1 equiv) and N,N-diisopropylethylamine (3.2 equiv) were added at 0° C. under nitrogen atmosphere. The resulting mixture was stirred for 5 minutes at 0° C., then 4-(difluoromethyl)-5-(4-morpholino-6-(piperazin-1-yl)-1,3,5-triazin-2-yl)pyrimidin-2-amine or the respective HCl salt (1.0 equiv) was added and the reaction was stirred at room temperature for 4-16 hours. After completion of the reaction, DMF was removed under high vacuum. The crude was dissolved in DCM and the organic layer was washed with deionized H₂O (2×) and an aqueous saturated Na₂CO₃-solution (3×). The organic layer was dried over Na₂SO₄, filtered and concentrated under reduced pressure. The crude product was purified by column chromatography on silica gel and then recrystallized from dichloromethane/pentane.

### Example 1

### 1-(4-(4-(2-amino-4-(difluoromethyl)pyrimidin-5-yl)-6-morpholino-1,3,5-triazin-2-yl)piperazin-1-yl)oct-7ene-1,6-dione

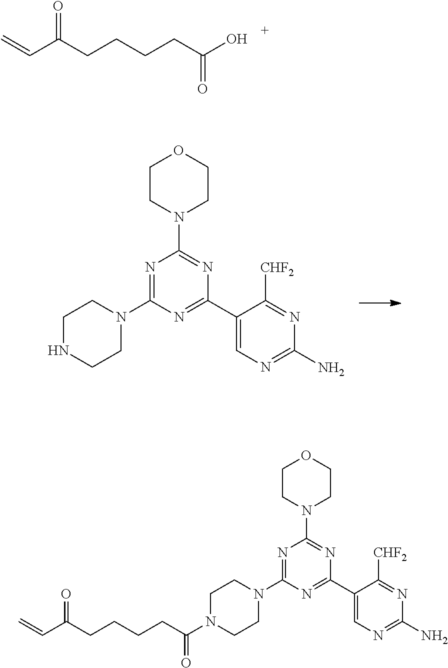 [SMILES: C=CC(=O)CCCCC(=O)O ; Nc1ncc(-c2nc(N3CCNCC3)nc(N3CCOCC3)n2)c(C(F)F)n1 ; C=CC(=O)CCCCC(=O)N1CCN(c2nc(-c3cnc(N)nc3C(F)F)nc(N3CCOCC3)n2)CC1]

1-(4-(4-(2-amino-4-(difluoromethyl)pyrimidin-5-yl)-6-morpholino-1,3,5-triazin-2-yl)piperazin-1-yl)oct -7-ene-1,6-dione was prepared according to general procedure 1 from 6-oxooct-7-enoic acid (163 mg, 1.04 mmol, 1.1 equiv). Purification by column chromatography on silica gel (dichloromethane/methanol/ammonia: 100:0:0→96:4:0.04) gave the desired compound as a colorless solid (73 mg, 0.14 mmol, 34%). HRMS (m/z): [M+H]⁺ calc. for C₂₄H₃₂F₂N₉O₃, 532.2591; found: 532.2593; [M+Na]+ calc. for C₂₄H₃₁F₂N₉NaO₃, 554.2410; found: 554.2415. HPLC: t_{R}=7.16 min (96.1% purity).

### Example 2

### (E)-1-(4-(4-(2-amino-4-(difluoromethyl)pyrimidin-5-yl)-6-morpholino-1,3,5-triazin-2-yl)piperazin-1-yl)non-7-ene-1,6-dione

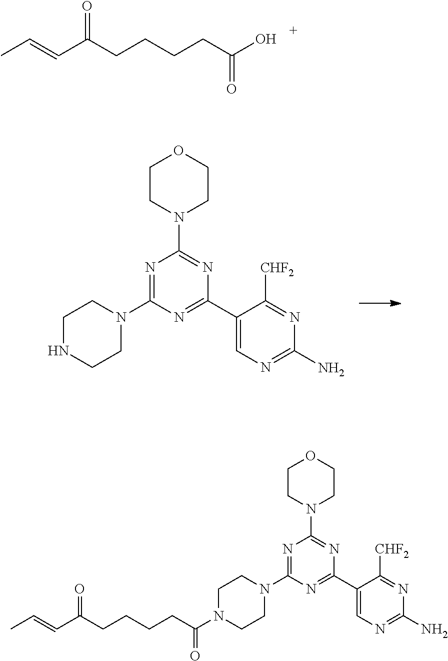 [SMILES: C/C=C/C(=O)CCCCC(=O)O ; Nc1ncc(-c2nc(N3CCNCC3)nc(N3CCOCC3)n2)c(C(F)F)n1 ; C/C=C/C(=O)CCCCC(=O)N1CCN(c2nc(-c3cnc(N)nc3C(F)F)nc(N3CCOCC3)n2)CC1]

(E)-1-(4-(4-(2-amino-4-(difluoromethyl)pyrimidin-5-yl)-6-morpholino-1,3,5-triazin-2-yl)piperazin-1-yl)non-7-ene-1,6-dione was prepared according to general procedure 1 from intermediate (E)-6-oxonon -7-enoic acid (130 mg, 0.76 mmol, 1.1 equiv). Purification by column chromatography on silica gel (dichloromethane/methanol/ammonia: 100:0:0→97:3:0.03) gave the desired compound as a colorless solid (90 mg, 0.16 mmol, 22%). HRMS (m/z): [M+H]⁺ calc. for C₂₅H₃₄F₂N₉O₃, 546.2747; found: 546.2750; [M+Na]+ calc. for C₂₅H₃₃F₂N₉NaO₃, 568.2567; found: 568.2570. HPLC: t_{R}=7.47 min (96.8% purity).

### Example 3

### 1-(4-(4-(2-amino-4-(difluoromethyl)pyrimidin-5-yl)-6-morpholino-1,3,5-triazin-2-yl)piperazin-1-yl)-8-methylnon-7-ene-1,6-dione

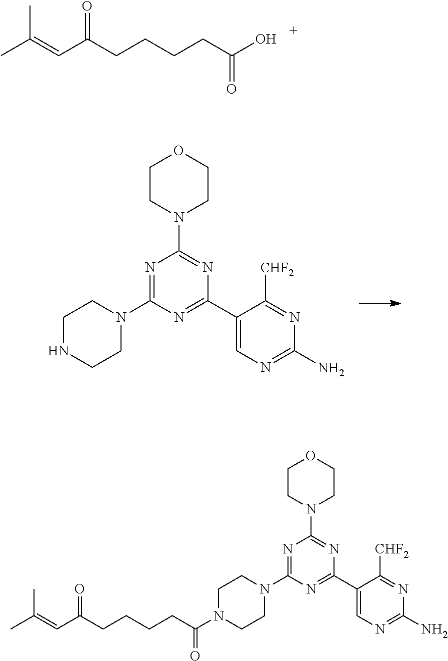 [SMILES: CC(C)=CC(=O)CCCCC(=O)O ; Nc1ncc(-c2nc(N3CCNCC3)nc(N3CCOCC3)n2)c(C(F)F)n1 ; CC(C)=CC(=O)CCCCC(=O)N1CCN(c2nc(-c3cnc(N)nc3C(F)F)nc(N3CCOCC3)n2)CC1]

1-(4-(4-(2-amino-4-(difluoromethyl)pyrimidin-5-yl)-6-morpholino-1,3,5-triazin-2-yl)piperazin-1-yl)-8-methylnon-7-ene-1,6-dione was prepared according to general procedure 1 from intermediate 8-methyl -6-oxonon-7-enoic acid (165 mg, 0.90 mmol, 1.1 equiv). Purification by column chromatography on silica gel (dichloromethane/methanol: 100:0→97:3) gave the desired compound as a colorless solid (65 mg, 0.12 mmol, 13%). HRMS (m/z): [M+H]⁺ calc. for C₂₆H₃₆F₂N₉O₃, 560.2904; found: 560.2906; [M+Na]+ calc. for C₂₆H₃₅F₂N₉NaO₃, 582.2723; found: 582.2726. HPLC: t_{R}=8.04 min (95.6% purity).

### Example 4

### N-(4-(4-(4-(2-amino-4-(difluoromethyl)pyrimidin-5-yl)-6-morpholino-1,3,5-triazin-2-yl)piperazin-1-yl)-4-oxobutyl)-N-methylacrylamide

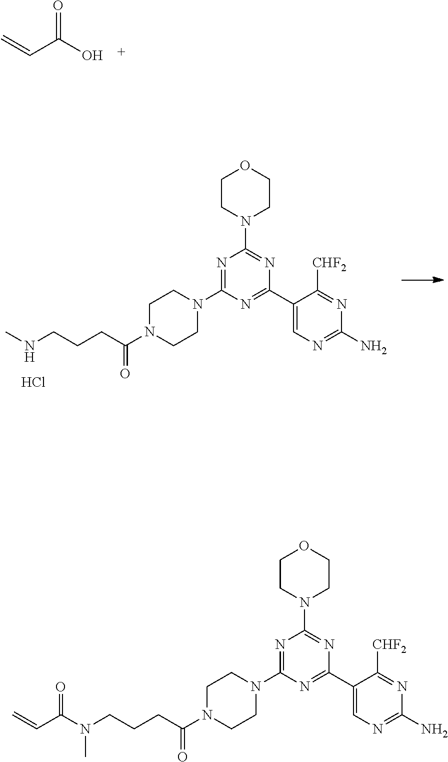

N-(4-(4-(4-(2-amino-4-(difluoromethyl)pyrimidin-5-yl)-6-morpholino-1,3,5-triazin-2-yl)piperazin-1-yl) -4-oxobutyl)-N-methylacrylamide was prepared according to general procedure 1 from 1-(4-(4-(2-amino -4-(difluoromethyl)pyrimidin-5-yl)-6-morpholino-1,3,5-triazin-2-yl)piperazin-1-yl)-4-(methylamino)butan -1-one (250 mg, 0.47 mmol, 1.0 equiv) and acrylic acid (34 mg, 32 μL, 0.47 mmol, 1.0 equiv). Purification by column chromatography on silica gel (dichloromethane/methanol/ammonia: 100:0:0→96:4:0.04) gave the desired compound as a colorless solid (90 mg, 0.16 mmol, 35%). HRMS (m/z): [M+H]⁺ calc. for C₂₄H₃₃F₂N₁₀O₃, 547.2700; found: 547.2704; [M+Na]+ calc. for C₂₄H₃₂F₂N₁₀NaO₃, 569.2519; found: 569.2525. HPLC: t_{R}=6.38 min (99.3% purity). The compound exists as a mixture of cis-trans amide bond rotamers.

### Example 5

### (E)-N-(4-(4-(4-(2-amino-4-(difluoromethyl)pyrimidin-5-yl)-6-morpholino-1,3,5-triazin-2-yl)piperazin-1-yl)-4-oxobutyl)-N-methylbut-2-enamide

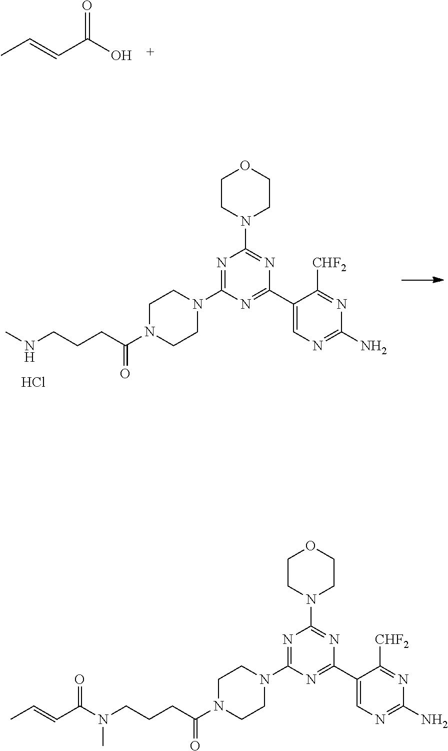

(E)-N-(4-(4-(4-(2-amino-4-(difluoromethyl)pyrimidin-5-yl)-6-morpholino-1,3,5-triazin-2-yl)piperazin -1-yl)-4-oxobutyl)-N-methylbut-2-enamide was prepared according to general procedure 1 from 1-(4-(4-(2-amino-4-(difluoromethyl)pyrimidin-5-yl)-6-morpholino-1,3,5-triazin-2-yl)piperazin-1-yl)-4-(methylamino)butan-1-one (250 mg, 0.47 mmol, 1.0 equiv) and crotonic acid (41 mg, 0.47 mmol, 1.0 equiv). Purification by column chromatography on silica gel (dichloromethane/methanol/ammonia: 100:0:0→96:4:0.04) gave the desired compound as a colorless solid (141 mg, 0.25 mmol, 53%). HRMS (m/z): [M+H]⁺ calc. for C₂₅H₃₅F₂N₁₀O₃, 561.2856; found: 561.2862; [M+Na]+ calc. for C₂₅H₃₄F₂N₁₀NaO₃, 583.2676; found: 583.2681. HPLC: t_{R}=6.68 min (97.3% purity). The compound exists as a mixture of cis-trans amide bond rotamers.

### Example 6

### N-(4-(4-(4-(2-amino-4-(difluoromethyl)pyrimidin-5-yl)-6-morpholino-1,3,5-triazin-2-yl)piperazin-1-yl)-4-oxobutyl)-N,3-dimethylbut-2-enamide

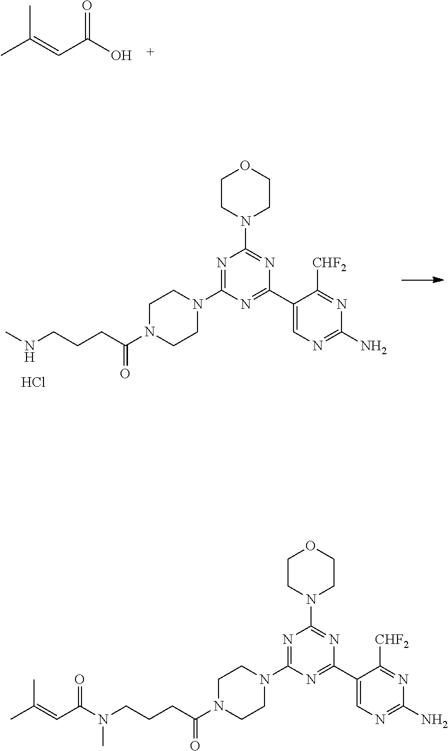

N-(4-(4-(4-(2-amino-4-(difluoromethyl)pyrimidin-5-yl)-6-morpholino-1,3,5-triazin-2-yl)piperazin-1-yl)-4-oxobutyl)-N,3-dimethylbut-2-enamide was prepared according to general procedure 1 from 1-(4-(4-(2-amino-4-(difluoromethyl)pyrimidin-5-yl)-6-morpholino-1,3,5-triazin-2-yl)piperazin-1-yl)-4-(methylamino)butan-1-one (250 mg, 0.47 mmol, 1.0 equiv) and 3-methylbut-2-enoic acid (47 mg, 0.47 mmol, 1.0 equiv). Purification by column chromatography on silica gel (dichloromethane/methanol/ammonia: 100:0:0→96:4:0.04) gave the desired compound as a colorless solid (133 mg, 0.23 mmol, 49%). HRMS (m/z): [M+Na]+ calc. for C₂₆H₃₆F₂N₁₀NaO₃, 597.2832; found: 597.2835. HPLC: t_{R}=6.98 min (97.4% purity). The compound exists as a mixture of cis-trans amide bond rotamers.

### Example 7

### 1-acryloyl-N-(2-(4-(4-(2-amino-4-(difluoromethyl)pyrimidin-5-yl)-6-morpholino-1,3,5-triazin-2-yl)piperazin-1-yl)-2-oxoethyl)piperidine-4-carboxamide

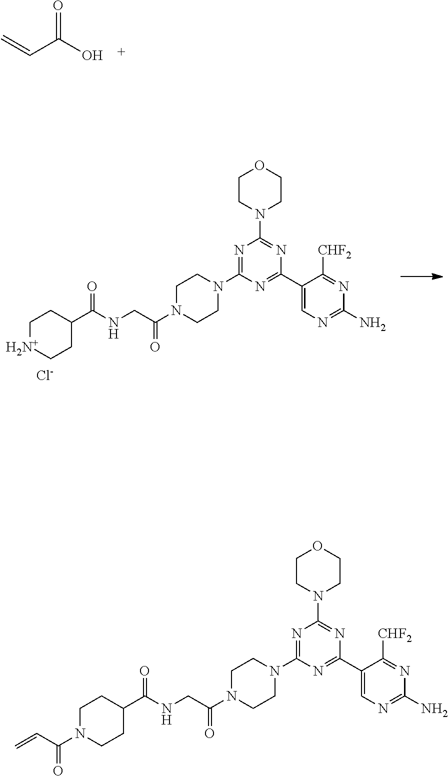

1-acryloyl-N-(2-(4-(4-(2-amino-4-(difluoromethyl)pyrimidin-5-yl)-6-morpholino-1,3,5-triazin-2-yl)piperazin-1-yl)-2-oxoethyl)piperidine-4-carboxamide was prepared according to general procedure 1 from 4-((2-(4-(4-(2-amino-4-(difluoromethyl)pyrimidin-5-yl)-6-morpholino-1,3,5-triazin-2-yl)piperazin-1-yl)-2-oxoethyl)carbamoyl)piperidin-1-ium chloride (250 mg, 0.42 mmol, 1.0 equiv). Purification by column chromatography on silica gel (dichloromethane/methanol/ammonia: 100:0:0→97:3:0.03) gave the desired compound as a colorless solid (67 mg, 0.11 mmol, 26%). HRMS (m/z): [M+H]⁺ calc. for C₂₇H₃₆F₂N₁₁O₄, 616.2914; found: 616.2912; [M+Na]+ calc. for C₂₇H₃₅F₂N₁₁NaO₄, 638.2734; found: 638.2730. HPLC: t_{R}=5.92 min (97.3% purity).

### Example 8

### 1-acryloyl-N-(2-(4-(4-(2-amino-4-(difluoromethyl)pyrimidin-5-yl)-6-morpholino-1,3,5-triazin-2-yl)piperazin-1-yl)-2-oxoethyl)-N-methylpiperidine-4-carboxamide

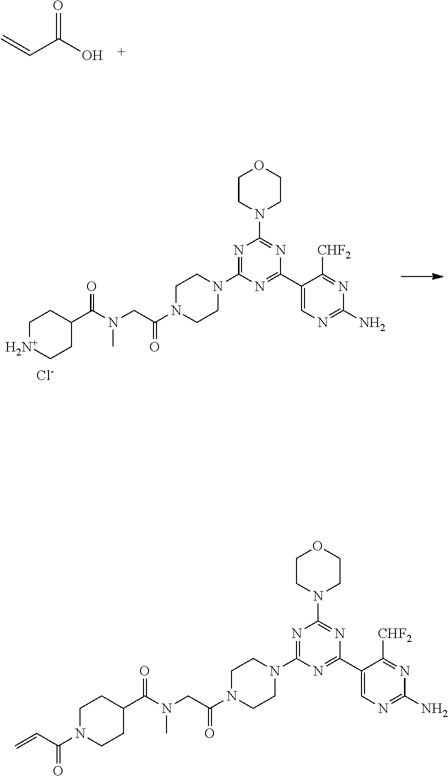

1-acryloyl-N-(2-(4-(4-(2-amino-4-(difluoromethyl)pyrimidin-5-yl)-6-morpholino-1,3,5-triazin-2-yl)piperazin-1-yl)-2-oxoethyl)-N-methylpiperidine-4-carboxamide was prepared according to general procedure 1 from 4-((2-(4-(4-(2-amino-4-(difluoromethyl)pyrimidin-5-yl)-6-morpholino-1,3,5-triazin-2-yl)piperazin-1-yl)-2-oxoethyl)(methyl)carbamoyl)piperidin-1-ium chloride (900 mg, 1.47 mmol, 1.0 eq.). Purification by column chromatography on silica gel (dichloromethane/methanol/ammonia: 100:0:0→92:8:0.08) gave the desired compound as a colorless solid (296 mg, 0.47 mmol, 32%). HRMS (m/z): [M+H]⁺ calc. for C₂₈H₃₈F₂N₁₁O₄, 630.3071; found: 630.3070; [M+Na]+ calc. for C₂₈H₃₇F₂N₁₁NaO₄, 652.2890; found: 652.2889. HPLC: t_{R}=6.03 min (98.6% purity).

### Example 9

### 4-(1-acryloylpiperidin-4-yl)-1-(4-(4-(2-amino-4-(difluoromethyl)pyrimidin-5-yl)-6-morpholino-1,3,5-triazin-2-yl)piperazin-1-yl)butan-1-one

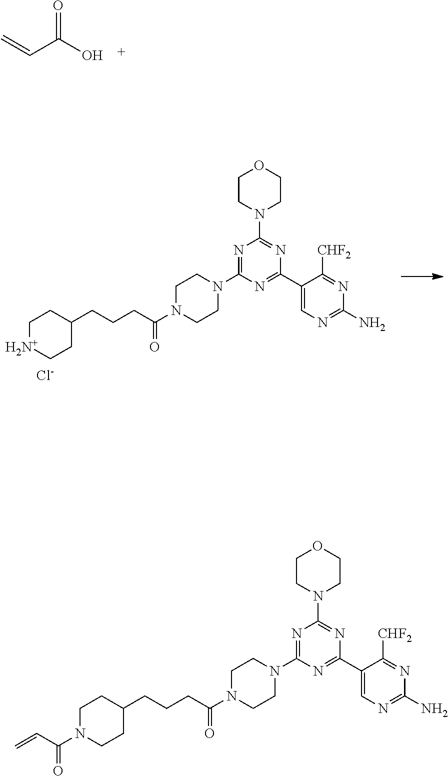

4-(1-acryloylpiperidin-4-yl)-1-(4-(4-(2-amino-4-(difluoromethyl)pyrimidin-5-yl)-6-morpholino-1,3,5-triazin-2-yl)piperazin-1-yl)butan-1-one was prepared according to general procedure 1 from 4-(4-(4-(4-(2-amino-4-(difluoromethyl)pyrimidin-5-yl)-6-morpholino-1,3,5-triazin-2-yl)piperazin-1-yl)-4-oxobutyl)piperidin-1-ium chloride (250 mg, 0.60 mmol, 1.0 equiv). Purification by column chromatography on silica gel (dichloromethane/methanol/ammonia: 100:0:0→97:3:0.03) gave the desired compound as a colorless solid (141 mg, 0.23 mmol, 39%). HRMS (m/z): [M+H]⁺ calc. for C₂₈H₃₉F₂N₁₀O₃, 601.3169; found: 601.3169; [M+Na]+ calc. for C₂₈H₃₈F₂N₁₀NaO₃, 623.2989; found: 623.2984. HPLC: t_{R}=7.22 min (99.0% purity).

### Example 10

### 1-(4-(4-(4-(2-amino-4-(difluoromethyl)pyrimidin-5-yl)-6-morpholino-1,3,5-triazin-2-yl)piperazine-1-carbonyl)piperidin-1-yl)-7-methyloct-6-ene-1,5-dione

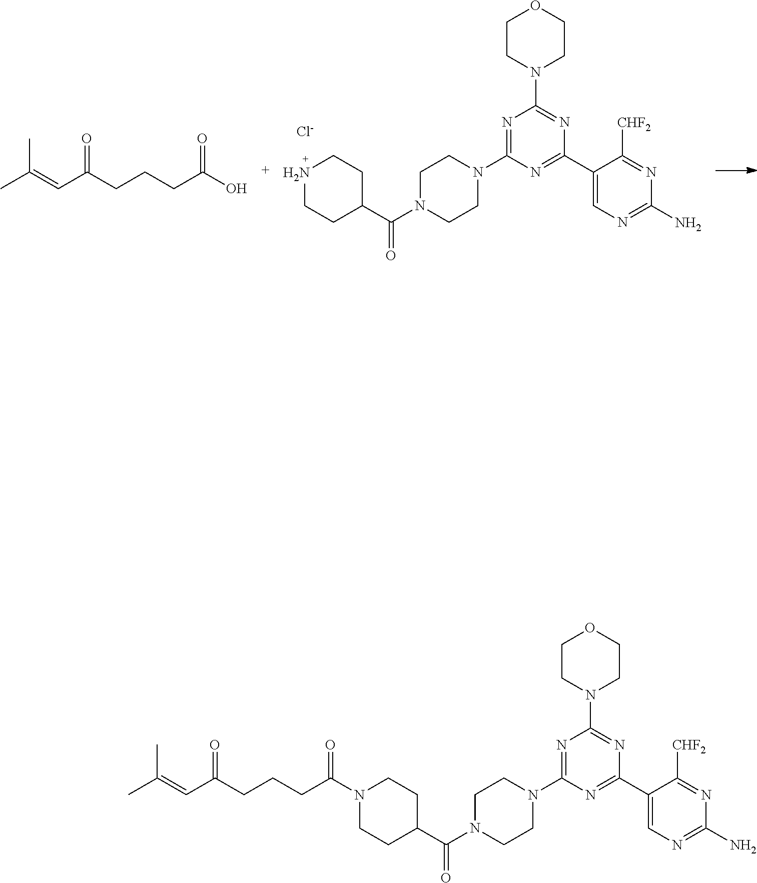 [SMILES: CC(C)=CC(=O)CCCC(=O)O ; [Cl-] ; Nc1ncc(-c2nc(N3CCOCC3)nc(N3CCN(C(=O)C4CCNCC4)CC3)n2)c(C(F)F)n1 ; CC(C)=CC(=O)CCCC(=O)N1CCC(C(=O)N2CCN(c3nc(-c4cnc(N)nc4C(F)F)nc(N4CCOCC4)n3)CC2)CC1]

1-(4-(4-(4-(2-amino-4-(difluoromethyl)pyrimidin-5-yl)-6-morpholino-1,3,5-triazin-2-yl)piperazine-1-carbonyl)piperidin-1-yl)-7-methyloct-6-ene-1,5-dione was prepared according to general procedure 1 from 4-(4-(4-(2-amino-4-(difluoromethyl)pyrimidin-5-yl)-6-morpholino-1,3,5-triazin-2-yl)piperazine-1-carbonyl)piperidin-1-ium chloride (200 mg, 0.37 mmol, 1.0 equiv) and 7-methyl-5-oxooct-6-enoic acid (63 mg, 0.11 mmol, 1.0 equiv). Purification by column chromatography on silica gel (dichloromethane/methanol/ammonia: 100:0:0→96:4:0.04) gave the desired compound as a colorless solid (70 mg, 0.062 mmol, 29%). HRMS (m/z): [M+H]⁺ calc. for C₃₁H₄₃F₂N₁₀O₄, 657.3431; found: 657.3425; [M+Na]+ calc. for C₃₁H₄₂F₂N₁₀NaO₄, 679.3251; found: 679.3253. HPLC: t_{R}=7.50 min (99.4% purity).

### Determination of Inhibitor Apparent Dissociation Constant

Apparent dissociation constants of compounds [Kᵢ₍ₐₚₚ₎] for p110α were determined by LanthaScreen Technology (Life Technologies), as described in Ref. ²⁵.

### Determination of Kinetic Constants

Maximum potential rate of covalent bond formation (k_{inact}) and dissociation constant of the first reversible binding (Kᵢ) of compounds for p110α were determined by LanthaScreen Technology (Life Technologies). The calculation of kinetic parameters was carried out through global fitting for numerical integration by using KinTek Global Kinetic Explorer modelling software²⁶⁻³¹.

### In Cell Western Cellular PI3K Signaling and IC₅₀ Determination

Protein phosphorylation was detected as follows: pSer473 of PKB/Akt with rabbit polyclonal antibody from Cell Signaling Technology (CST) (#4058) by In-Cell Western assays, where 1.2×10⁴ SKOV3 cells/well in 96-well plates were plated (Cell Carrier, PerkinElmer) for 24 hours (37° C., 5% CO₂), as described in Ref. ²⁵.

### NanoBRET Target Engagement Assays

N-terminal NanoLuc fused PI3K were encoded in pFN31K expression vector (Promega), including flexible Gly-Ser-Ser-Gly-Ala-Ile-Ala linkers between NanoLuc and each full-length of target kinases including PI3Kα, PI3Kα C862S, PI3Kβ, and PI3Kδ. HEK293 cells were co-transfected with NanoLuc/PI3K and its regulatory subunit p85 at a mass ratio 1:10 using jetPEI transfection reagent (Polyplus transfection, #101B-010N).

TABLE 1
Intrinsic reactivity and in vitro data for some of the compounds of the invention and CNX-1351.

| | | | | [symbol] | | [symbol] | | | |
|---|---|---|---|---|---|---|---|---|---|
| | [symbol] | [symbol] | | PI3Kα, | | PI3Kα, | | [symbol] | |
| Comp. | [symbol] | [M⁻¹ · s [symbol] | ^{$}SD | [s⁻¹] | ^{$}SD | [nM] | SD | [nM⁻¹ · s⁻¹] | SD |
| 19 | 2.73 | 4.84 × 10⁻² | 1.24 × 10⁻³ | 2.00 × 10⁻⁴ | 8.07 × 10⁻⁵ | 1.51 | 0.16 | 1.30 × 10⁻⁴ | 4.04 × 10⁻⁵ |
| 20 | 3.13 | 8.06 × 10⁻² | 2.98 × 10⁻³ | 1.21 × 10⁻³ | 2.65 × 10⁻⁵ | 6.69 | 0.78 | 1.83 × 10⁻⁴ | 2.42 × 10⁻⁵ |
| 2 | 3.53 | 8.57 × 10⁻² | 8.33 × 10⁻³ | 3.92 × 10⁻³ | 2.64 × 10⁻⁴ | 5.76 | 0.83 | 6.93 × 10⁻⁴ | 1.31 × 10⁻⁴ |
| 21 | 3.91 | 6.32 × 10⁻² | 4.40 × 10⁻³ | 1.60 × 10⁻² | 1.11 × 10⁻³ | 11.72 | 2.29 | 1.27 × 10⁻³ | 9.46 × 10⁻⁵ |
| 22 | 2.89 | 1.88 × 10⁻⁴ | 1.18 × 10⁻⁵ | 1.13 × 10⁻⁵ | 3.51 × 10⁻⁶ | 3.48 | 0.28 | 3.25 × 10⁻⁶ | 1.38 × 10⁻⁷ |
| 23 | 3.28 | 2.24 × 10⁻⁴ | 1.60 × 10⁻⁵ | 2.56 × 10⁻⁵ | 5.56 × 10⁻⁷ | 8.06 | 0.81 | 3.21 × 10⁻⁶ | 5.61 × 10⁻⁷ |
| 5 | 3.68 | 2.20 × 10⁻⁴ | 1.66 × 10⁻⁵ | 1.87 × 10⁻⁴ | 6.65 × 10⁻⁵ | 10.11 | 0.99 | 1.87 × 10⁻⁵ | 2.63 × 10⁻⁶ |
| 24 | 4.08 | 1.73 × 10⁻⁴ | 4.84 × 10⁻⁶ | 9.03 × 10⁻⁴ | 7.13 × 10⁻⁵ | 15.73 | 2.81 | 5.82 × 10⁻⁵ | 6.57 × 10⁻⁶ |
| 28 | 1.72 | 4.10 × 10⁻⁴ | 1.10 × 10⁻⁵ | 5.77 × 10⁻⁵ | 6.46 × 10⁻⁶ | 10.84 | 1.07 | 5.39 × 10⁻⁶ | 1.09 × 10⁻⁶ |
| 35 | 1.89 | 2.49 × 10⁻⁴ | 1.20 × 10⁻⁵ | 9.84 × 10⁻⁵ | 1.28 × 10⁻⁵ | 10.76 | 1.14 | 9.29 × 10⁻⁶ | 2.15 × 10⁻⁶ |
| 40 | 2.28 | 3.07 × 10⁻⁴ | 2.33 × 10⁻⁵ | 2.92 × 10⁻⁴ | 1.01 × 10⁻⁵ | 8.56 | 0.59 | 3.43 × 10⁻⁵ | 3.66 × 10⁻⁶ |
| 43 | 0.65 | 3.70 × 10⁻⁴ | 1.28 × 10⁻⁵ | 1.20 × 10⁻³ | 2.52 × 10⁻⁵ | 2.94 | 0.38 | 4.14 × 10⁻⁴ | 4.99 × 10⁻⁵ |
| 48 | 0.83 | 4.52 × 10⁻⁴ | 1.69 × 10⁻⁵ | 2.53 × 10⁻⁴ | 2.35 × 10⁻⁵ | 14.06 | 1.71 | 1.80 × 10⁻⁵ | 5.94E × 10⁻⁷ |
| 51 | 1.08 | 4.37 × 10⁻⁴ | 3.79 × 10⁻⁶ | 1.95 × 10⁻⁴ | 2.96 × 10⁻⁵ | 10.65 | 1.38 | 1.88 × 10⁻⁵ | 5.13 × 10⁻⁶ |
| 54 | 0.89 | 3.54 × 10⁻⁴ | 5.93 × 10⁻⁶ | 1.08 × 10⁻³ | 1.06 × 10⁻⁴ | 5.15 | 0.18 | 2.10 × 10⁻⁴ | 1.97 × 10⁻⁵ |
| 57 | 1.07 | 3.88 × 10⁻⁴ | 1.77 × 10⁻⁵ | 3.78 × 10⁻⁴ | 5.35 × 10⁻⁵ | 14.58 | 0.86 | 2.61 × 10⁻⁵ | 5.38 × 10⁻⁶ |
| 58 | 1.33 | 3.86 × 10⁻⁴ | 6.72 × 10⁻⁶ | 4.06 × 10⁻⁴ | 4.05 × 10⁻⁵ | 12.20 | 2.13 | 3.36 × 10⁻⁵ | 3.03 × 10⁻⁶ |
| 44 | 0.52 | — | — | 1.26 × 10⁻¹² | 1.16 × 10⁻¹⁴ | 2.55 | 0.23 | 4.98 × 10⁻¹³ | 4.41 × 10⁻¹⁴ |
| 55 | 0.77 | — | — | 1.24 × 10⁻¹² | 1.71 × 10⁻¹³ | 6.16 | 0.77 | 2.02 × 10⁻¹³ | 2.32 × 10⁻¹⁵ |
| 62 | 1.46 | 5.18 × 10⁻⁴ | 2.01 × 10⁻⁵ | 1.87 × 10⁻⁴ | 2.02 × 10⁻⁵ | 16.2 | 0.62 | 1.16 × 10⁻⁵ | 1.61 × 10⁻⁶ |
| 63 | 1.16 | 2.67 × 10⁻⁴ | 1.56 × 10⁻⁵ | 2.14 × 10⁻⁴ | 2.34 × 10⁻⁵ | 33.6 | 3.34 | 6.35 × 10⁻⁶ | 7.96 × 10⁻⁸ |
| 41 | 2.68 | 2.25 × 10⁻⁴ | 2.44 × 10⁻⁶ | 5.04 × 10⁻⁴ | 8.60 × 10⁻⁵ | 7.15 | 0.59 | 7.03 × 10⁻⁵ | 9.31 × 10⁻⁶ |
| 61 | 1.47 | 2.90 × 10⁻⁴ | 1.41 × 10⁻⁵ | 1.02 × 10⁻³ | 2.08 × 10⁻⁵ | 4.29 | 0.29 | 2.39 × 10⁻⁴ | 1.60 × 10⁻⁵ |
| 100 | 3.85 | 3.24 × 10⁻⁴ | 7.74 × 10⁻⁶ | 1.23 × 10⁻⁴ | 8.54 × 10⁻⁶ | 9.11 | 1.07 | 1.37 × 10⁻⁵ | 2.60 × 10⁻⁶ |
| 101 | 3.85 | 2.90 × 10⁻⁴ | 2.18 × 10⁻⁵ | 1.50 × 10⁻⁴ | 1.52 × 10⁻⁵ | 10.5 | 0.63 | 1.43 × 10⁻⁵ | 1.85 × 10⁻⁶ |
| 118 | 1.78 | — | — | 1.08 × 10⁻² | 1.52 × 10⁻⁴ | 1.00 | 0.14 | 1.08 × 10⁻² | 1.34 × 10⁻³ |
| 1 | — | >>1.00 × 10⁻¹ | — | 245 × 10⁻⁴ | 16.6 × 10⁻⁴ | 5.87 | 0.23 | 420 × 10⁻⁵ | 62.2 × 10⁻⁵ |
| 7 | — | 2.28 × 10⁻⁴ | 8.42 × 10⁻⁶ | 0.69 × 10⁻⁴ | 0.02 × 10⁻⁴ | 7.22 | 0.25 | 0.96 × 10⁻⁵ | 0.05 × 10⁻⁵ |
| 9 | — | 3.35 × 10⁻⁶ | 1.13 × 10⁻⁶ | — | — | 5.40^{d} | 0.20 | — | — |
| 11 | — | <<1.00 × 10⁻⁷ | — | — | — | 6.95^{d} | 0.63 | — | — |
| 13 | — | 1.38 × 10⁻⁴ | 1.00 × 10⁻⁵ | 0.76 × 10⁻⁴ | 0.07 × 10⁻⁴ | 5.36 | 0.72 | 1.42 × 10⁻⁵ | 0.10 × 10⁻⁵ |
| 15 | — | 6.99 × 10⁻⁷ | 2.04 × 10⁻⁷ | — | — | — | — | — | — |
| 17 | — | <<1.00 × 10⁻⁷ | — | — | — | — | — | — | — |
| CNX-1351 | 4.68 | 2.22 × 10⁻⁴ | 4.05 × 10⁻⁵ | 6.57 × 10⁻⁴ | 4.05 × 10⁻⁴ | 38.09 | 3.18 | 1.74 × 10⁻⁵ | 2.08 × 10⁻⁶ |

[symbol]

20.9 was used for calculation of [symbol] (partition coefficient). [symbol]
reactivity of the inhibitors measured with HPLC (1 mM inhibitor + 600 mM βME; n = 3).
^{$}each mean and SD were calculated from 3 independent measurements. [symbol]
mean and SD were calculated from 2 independent measurements. These compounds did not show covalent behavior due to the poor electrophilicity of their warheads.
Kᵢ value was determined after 60 min of drug incubation with PI3Kα.

TABLE 2
Cellular data for some of the compounds
of the invention and CNX-1351.

| | IC₅₀ pPKB (S473) | |
|---|---|---|
| Comp. | SKOV3, [nM] | ^{$}SD |
| 56 | 891 | — |
| 19 | 20.04 | 1.82 |
| 20 | 38.79 | 3.89 |
| 2 | 46.9 | 5.03 |
| 21 | 44.49 | 9.25 |
| 5 | 52.81 | 15.85 |
| 24 | 89.18 | 10.75 |
| 28 | 51 | 4.6 |
| 35 | 47 | 5.7 |
| 40 | 48 | 6.1 |
| 43 | 82 | 2.7 |
| 48 | 664 | 78 |
| 51 | 686 | 67 |
| 54 | 86 | 2.9 |
| 57 | 250 | 27 |
| 58 | 260 | 27 |
| 44 | 200 | 43 |
| 55 | 227 | 22 |
| 62 | 82 | 14 |
| 63 | 41 | 4.4 |
| 41 | 24 | 1.5 |
| 61 | 23 | 2.5 |
| 100 | 56 | 5.4 |
| 101 | 43 | 8.0 |
| 69 | 31 | 2.4 |
| 70 | 63 | 3.3 |
| 71 | 112 | 5.5 |
| 72 | 46 | 0.24 |
| 76 | 43 | 1.7 |
| 78 | 177 | 0.63 |
| 81 | 24 | 1.9 |
| 86 | 17 | 0.55 |
| 84 | 11 | 1.2 |
| 85 | 73 | 3.5 |
| 89 | 6.7 | 0.06 |
| 90 | 93 | 3.9 |
| 92 | 34 | 0.44 |
| 95 | 33 | 2.3 |
| 94 | 12 | 1.9 |
| 97 | 17 | 0.50 |
| 118 | 5.96 | 0.36 |
| CNX-1351 | 164 | 18 |

^{$}each mean and SD were calculated from 2 or 3 independent measurements.

TABLE 3
Experimental LogD and PBS solubility for some
of the compounds of the invention and CNX-1351.

| | Experimental LogD, | PBS Solubility, |
|---|---|---|
| Comp. | pH 7.4^{$} | pH 7.4, μM^{$} |
| 62 | 1.40 | 393 |
| 63 | 1.79 | 363 |
| 41 | 2.67 | 243 |
| 61 | 1.70 | 381 |
| 100 | 3.32 | 7 |
| 101 | 2.66 | 46 |
| CNX-1351 | 3.96 | 10 |

^{$}calculated from 3 independent measurements.

### On- and Off-Target Effects

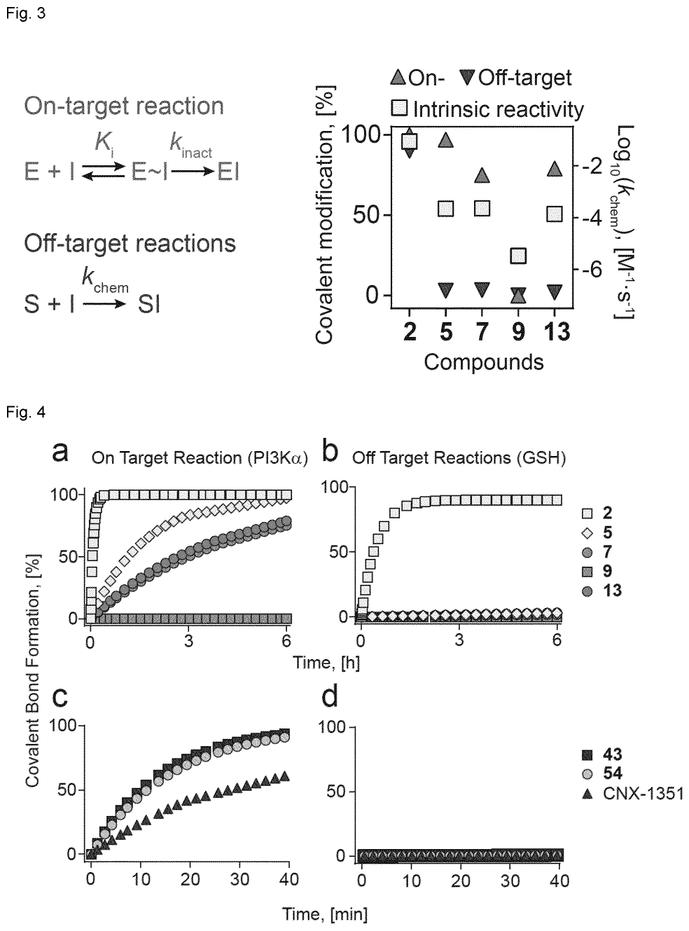

To estimate on-target and off-target reactivity, the reaction of the warhead with Cys862 of PI3Kα or cellular thiols was modelled using KinTek software using the two competitive on-target and off target reactions depicted in FIG. 3. The on-target model involves the inhibitor dissociation constant Ki for the formation of the reversible enzyme-inhibitor complex (E˜I) and the rate of the covalent reaction with the protein (k_{inact}; for the measurement of Kᵢ and k_{inact} see section above) to form the irreversible inhibitor complex (EI).

The results of this modelling (FIG. 3 and FIG. 4) illustrate that i) highly reactive electrophiles (2) react very rapidly with the target, but are mostly consumed by off-target reactions; (ii) moderate electrophiles (5, 7, 13) selectively engage the target and show negligible side reactions; and (iii) minimally reactive molecules (9) engage neither the desired target nor undesired cellular thiols.

This defines a range for k_{chem} from ˜5·10⁻⁵ to 5·10^{-3 [}M⁻¹s⁻¹] for warheads with an acceptable off-target activity. On-target reactivity is, however, promoted by reversible affinity and warhead proximity to the targeted nucleophilic amino acid side chain.

### REFERENCES

25. Bohnacker, T.; Prota, A. E.; Beaufils, F.; Burke, J. E.; Melone, A.; Inglis, A. J.; Rageot, D.; Sele, A. M.; Cmiljanovic, V.; Cmiljanovic, N.; Bargsten, K.; Aher, A.; Akhmanova, A.; Diaz, J. F.; Fabbro, D.; Zvelebil, M.; Williams, R. L.; Steinmetz, M. O.; Wymann, M. P., Deconvolution of Buparlisib's mechanism of action defines specific PI3K and tubulin inhibitors for therapeutic intervention. Nat Commun 2017, 8, 14683.
26. Choi, B.; Rempala, G. A.; Kim, J. K., Beyond the Michaelis-Menten equation: Accurate and efficient estimation of enzyme kinetic parameters. Sci Rep 2017, 7 (1), 17018.
27. Janes, M. R.; Zhang, J.; Li, L. S.; Hansen, R.; Peters, U.; Guo, X.; Chen, Y.; Babbar, A.; Firdaus, S. J.; Darjania, L.; Feng, J.; Chen, J. H.; Li, S.; Li, S.; Long, Y. O.; Thach, C.; Liu, Y.; Zarieh, A.; Ely, T.; Kucharski, J. M.; Kessler, L. V.; Wu, T.; Yu, K.; Wang, Y.; Yao, Y.; Deng, X.; Zarrinkar, P. P.; Brehmer, D.; Dhanak, D.; Lorenzi, M. V.; Hu-Lowe, D.; Patricelli, M. P.; Ren, P.; Liu, Y., Targeting KRAS Mutant Cancers with a Covalent G12C-Specific Inhibitor. Cell 2018, 172 (3), 578-589.e17.
28. Johnson, K. A., Fitting enzyme kinetic data with KinTek Global Kinetic Explorer. Methods Enzymol 2009, 467, 601-626.
29. Müller, M. P.; Jeganathan, S.; Heidrich, A.; Campos, J.; Goody, R. S., Nucleotide based covalent inhibitors of KRas can only be efficient in vivo if they bind reversibly with GTP-like affinity. Sci Rep 2017, 7 (1), 3687.
30. Schwartz, P. A.; Kuzmic, P.; Solowiej, J.; Bergqvist, S.; Bolanos, B.; Almaden, C.; Nagata, A.; Ryan, K.; Feng, J.; Dalvie, D.; Kath, J. C.; Xu, M.; Wani, R.; Murray, B. W., Covalent EGFR inhibitor analysis reveals importance of reversible interactions to potency and mechanisms of drug resistance. Proc Natl Acad Sci USA 2014, 111 (1), 173-8.
31. Johnson, K. A.; Simpson, Z. B.; Blom, T., Global kinetic explorer: a new computer program for dynamic simulation and fitting of kinetic data. Anal Biochem 2009, 387 (1), 20-9.
80. Montero, D.; Tachibana, C.; Rahr Winther, J.; Appenzeller-Herzog, C., Intracellular glutathione pools are heterogeneously concentrated. Redox Biol 2013, 1 (1), 508-13. doi:10.1016/j.redox.2013.10.005

1. A compound of formula (IV) or a prodrug, metabolite, tautomer, solvate or pharmaceutically acceptable salt thereof, 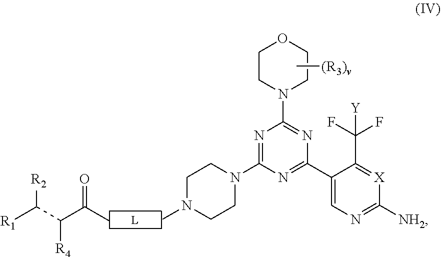
wherein
X is CH or N,
Y is H or F,
R₁ and R₂ are independently of each other selected from H, CH₃, cyclopropyl, —F, —CH₂—F, —CH₂—CH₂—F, —CN, 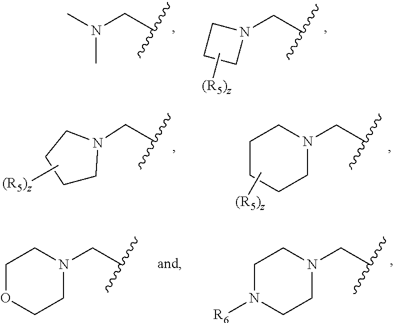
with R₅ being F or CH₃, R₆ being C₁₋₆-alkyl and z being 0, 1 or 2,
R₃ is C₁₋₃-alkyl or two residues R₃ form a bridge —(CH₂)ᵣ— with r being 1, 2 or 3, particularly R₃ is C₁₋₃-alkyl, more particularly CH₃,
v is 0, 1, 2, 3 or 4, particularly 0, 1 or 2, even more particularly 0 or 1,
R₄ is H, F or —CN, particularly H.
L is a linker moiety composed of C, N, O and/or H atoms, wherein the linker moiety L has a length between 3 Å and 15 Å, particularly between 7 Å and 14 Å, more particularly between 10 Å and 13 Å, even more particularly between 10.5 Å and 12.5 Å,
the dashed line represents a carbon-carbon single bond or carbon-carbon double bond, particularly a carbon-carbon double bond.

2. The compound according to claim 1, wherein L consists of 2 to 5 moieties independently of each other selected from C₁₋₅-alkyl, —CO—, —NH—, —N(CH₃)—, —O—, phenyl,

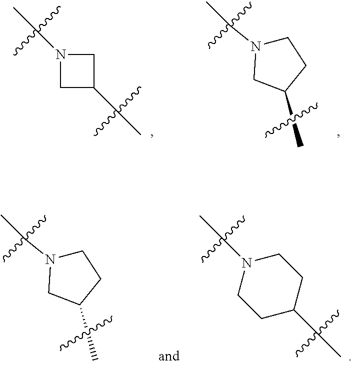

3. The compound according to claim 1, wherein L is a moiety selected from
—U-L₁-W—,
-C₁₋₂-alkyl-CO-L₂-(CH₂)ₚ—W—, 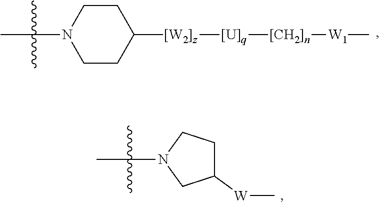
wherein
U is —CH₂— or —NH— or —N(CH₃)— or —O— or phenyl
L₁ is C₁-C₄-alkyl,
L₂ is azetidine, pyrrolidine or piperidine,
W, W₁ and W₂ are independently of each other —CO— or —CH₂—,
n is 0, 1, 2 or 3,
p, q and z are independently of each other 0 or 1.

4. The compound according to claim 1, wherein L is a moiety selected from
—U-L₁-W—, 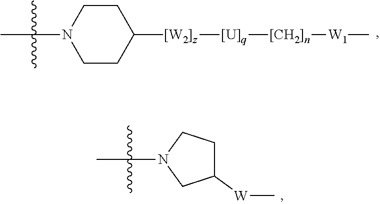
wherein
U is —CH₂— or —NH— or —N(CH₃)— or —O— or phenyl, particularly —NH— or —N(CH₃)—, more particularly —NH—,
L₁ is C₁-C₄-alkyl,
L₂ is azetidine, pyrrolidine or piperidine,
W, W₁ and W₂ are independently of each other —CO— or —CH₂—,
n is 0, 1, 2 or 3,
p, q and z are independently of each other 0 or 1.

5. The compound according to claim 1, wherein R₁ and R₂ are independently of each other selected from H, CH₃, 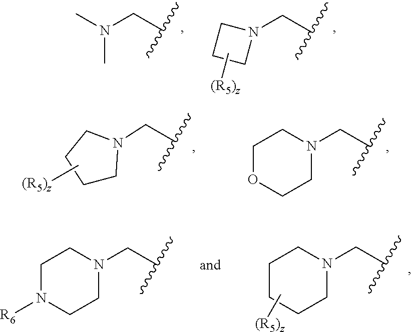
particularly from H, CH₃, 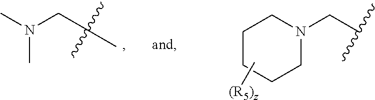
with R₅ being F or CH₃, R₆ being C₁₋₆-alkyl and z being 0, 1 or 2.

6. The compound according to claim 1, wherein R₂ is cyclopropyl and R₄ is —CN.

7. The compound according to claim 1, wherein the compound is a compound of formula (Ia), particularly of formula (I), 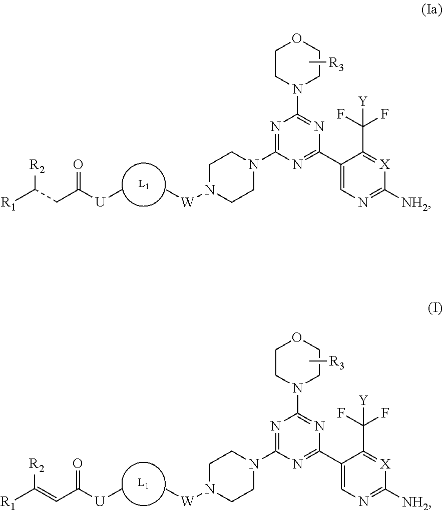
wherein
X is CH or N,
Y is H or F,
W is —CO— or —CH₂—,
U is —CH₂— or —NH— or —N(CH₃)—,
R₁ and R₂ are independently of each other selected from H, CH₃, 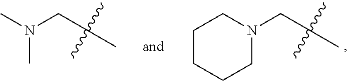
particularly R₁ and R₂ are independently of each other H or —CH₃.
L₁ is C₁-C₄-alkyl,
R₃ is H or —CH₃.
the dashed line represents a carbon-carbon single bond or carbon-carbon double bond.)

8. The compound according to claim 7, wherein R¹ and R² are H.

9. The compound according to claim 7, wherein W is —CO— and U is —CH₂—.

10. The compound according to claim 1, wherein the compound is a compound of formula (IIa), particularly of formula (II), 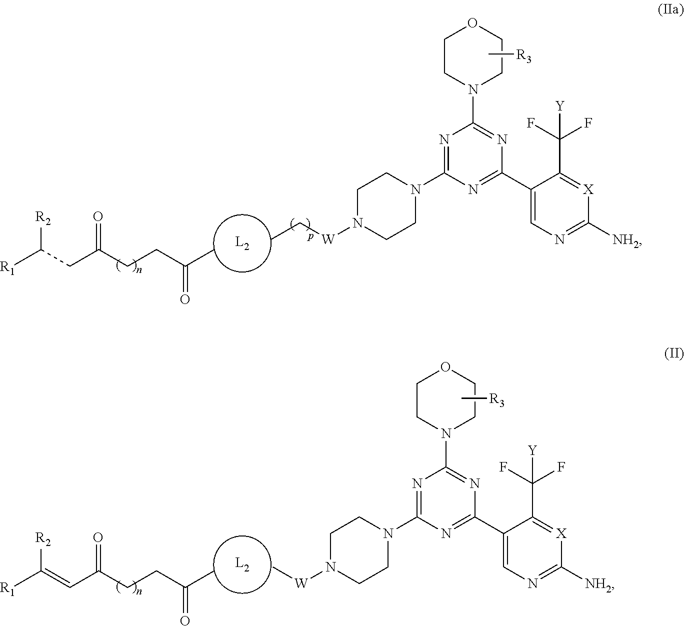
wherein
X is CH or N,
Y is H or F,
W is CO or CH₂.
L₂ is azetidine or pyrrolidine or piperidine, particularly 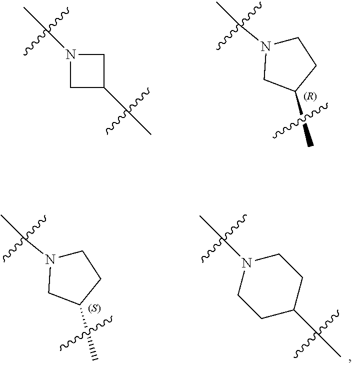
R₁ and R₂ are independently of each other selected from H, CH₃, 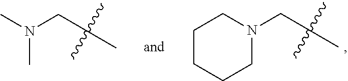
particularly R₁ and R₂ are independently of each other selected from H and CH₃,
R₃ is H or CH₃,
n is equal to 1 or 2,
p is equal to 0 or 1,
the dashed line represents a carbon-carbon single bond or carbon-carbon double bond.

11. The compound according to claim 1, wherein the compound is a compound of formula (IIIa), particularly of formula (III), 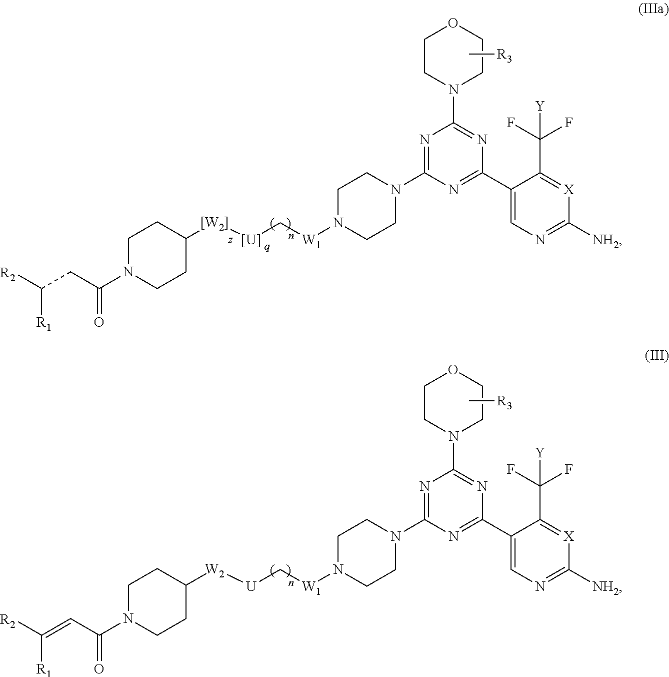
wherein
X is CH or N,
Y is H or F,
W₁ and W₂ are independently of each other —CO— or —CH₂—.
U is —CH₂— or —NH— or —N(CH₃)— or —O— or phenyl,
R₁ and R₂ are independently of each other selected form H, CH₃, 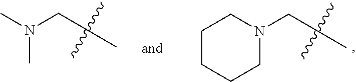
R₃ is H or CH₃,
n is equal to 0 or 1 or 2 or 3,
z is equal to 0 or 1
q is equal to 0 or 1,
the dashed line represents a carbon-carbon single bond or carbon-carbon double bond.

12. The compound according to claim 11, wherein R¹ and R² are H.

13. The compound according to claim 11, wherein W₁ is —CO— and U is —O— or —N(CH₃)—.

14. The compound according to claim 1, for use in the treatment of a disease, particularly a disease caused by an activating mutation of the PI3KCA gene or activation of a class I PI3K, in particular PI3Kα.

15. The compound according to claim 1, for use in the treatment of tumor disease, overgrowth syndrome, neurological disease, immunological disease, particularly wherein the tumor is a solid tumor and/or the tumor disease is selected from lymphoma and leukemia.

16. An intermediate of formula (V), 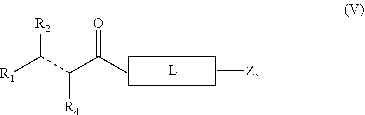
wherein
R₁, R₂, R₄ and L are defined as described above.
Z is —OH, Br, COOH or NH₂.
